SEQUENCE LISTING (65 sequences)

SEQ ID NO: 1 - DNA, 1992 bp, Artificial Sequence
  SARS-CoV-2-S1 immunogen
atgttcgtgttcctggtcctgctcccactggtgtcctcgcagtgcgtgaacctgaccacccgcacccagctgccccctgcgtacaccaactccatcaccaggggcgtctactatcccgacaaggtgtttcgaagttccgttctgcactcgacccaagacctgttcctccccttcttcagcaacgtgacctggttccatgctatccacgtatcgggtacgaacgggaccaagcgcttcgacaaccccgtcctgcccttcaacgacggcgtgtactttgcatccaccgagaagtcgaacattatcagggggtggattttcggcaccaccctagactcgaagacacagtcgctgctgatcgtgaacaacgcgaccaatgtcgtcatcaaggtttgcgaattccagttctgcaatgaccccttcctgggagtctactaccacaagaacaacaagagctggatggagagtgagttcagagtgtattcctccgctaacaattgcaccttcgagtacgtatcccagcccttcctgatggacctggagggcaagcaggggaactttaaaaatctgcgcgagttcgtgtttaagaacatcgacggctacttcaagatctatagtaagcacacacccataaatctggtccgcgacctgccgcaaggcttctccgctttggaacccctcgtggacctgccgattgggatcaacatcacacgttttcagactctcctggccctgcaccggagttacctgacacccggtgactcctcctcaggctggactgccggggccgccgcttactacgtcggctatttgcagcccagaactttcctcctgaagtataacgagaacggcacgatcaccgatgccgtggattgtgcgctggacccgctgtcggagacaaagtgcacgctgaagtcgtttaccgtggagaaggggatctaccagacatctaacttccgggttcagcccactgagtccatcgtgcgcttccccaacatcacgaatctctgccccttcggcgaggtgttcaacgcaactcgcttcgccagtgtgtacgcctggaaccgcaagcggatttccaattgtgtcgctgactacagtgtgctttacaactctgcctcattcagtacgttcaagtgttacggcgtgtccccgaccaagctgaatgatctgtgcttcaccaacgtatacgccgactcgtttgtcatccgcggcgatgaagtgaggcagatcgcgcccggccagacgggcaagatcgccgactacaactacaagctgcccgatgatttcactggctgtgttattgcttggaatagtaacaatctggactcgaaggtcggggggaactacaactatctctataggctcttcagaaaaagtaatctgaagcctttcgagcgcgacatctcgactgagatatatcaggcaggctccaccccctgcaacggcgtggaggggtttaattgctacttcccccttcagagctacggctttcagccaacaaacggcgtaggctatcagccctaccgcgtggtggtgctgtcattcgaactcctgcacgcccccgcaaccgtctgcggccctaagaagagtacgaacctggtgaagaacaagtgcgtcaacttcaatttcaacggcctcaccggcacaggggtgctgaccgagtccaacaagaagtttctcccgttccagcagtttgggagggacatcgcagacactaccgacgcggtgagggacccacagaccttggagatactggacatcactccttgcagcttcgggggcgtgtcggtcataactcccggcactaacacctcaaaccaggtcgccgtgctctaccaggacgtgaactgtaccgaggtgcccgtggcgattcacgccgaccagctgacacccacgtggagggtgtactccaccggaagtaatgtcttccagacccgcgccgggtgtctgataggcgccgagcacgtcaacaactcgtacgagggatcctga

SEQ ID NO: 2 - protein, 663 aa, Artificial Sequence
  SARS-CoV-2-S1 immunogen
MFVFLVLLPLVSSQCVNLTTRTQLPPAYTNSITRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYEGS

SEQ ID NO: 3 - DNA, 2010 bp, Artificial Sequence
  SARS-CoV-2-S1H immunogen
atgttcgtgttcctggtcctgctcccactggtgtcctcgcagtgcgtgaacctgaccacccgcacccagctgccccctgcgtacaccaactccatcaccaggggcgtctactatcccgacaaggtgtttcgaagttccgttctgcactcgacccaagacctgttcctccccttcttcagcaacgtgacctggttccatgctatccacgtatcgggtacgaacgggaccaagcgcttcgacaaccccgtcctgcccttcaacgacggcgtgtactttgcatccaccgagaagtcgaacattatcagggggtggattttcggcaccaccctagactcgaagacacagtcgctgctgatcgtgaacaacgcgaccaatgtcgtcatcaaggtttgcgaattccagttctgcaatgaccccttcctgggagtctactaccacaagaacaacaagagctggatggagagtgagttcagagtgtattcctccgctaacaattgcaccttcgagtacgtatcccagcccttcctgatggacctggagggcaagcaggggaactttaaaaatctgcgcgagttcgtgtttaagaacatcgacggctacttcaagatctatagtaagcacacacccataaatctggtccgcgacctgccgcaaggcttctccgctttggaacccctcgtggacctgccgattgggatcaacatcacacgttttcagactctcctggccctgcaccggagttacctgacacccggtgactcctcctcaggctggactgccggggccgccgcttactacgtcggctatttgcagcccagaactttcctcctgaagtataacgagaacggcacgatcaccgatgccgtggattgtgcgctggacccgctgtcggagacaaagtgcacgctgaagtcgtttaccgtggagaaggggatctaccagacatctaacttccgggttcagcccactgagtccatcgtgcgcttccccaacatcacgaatctctgccccttcggcgaggtgttcaacgcaactcgcttcgccagtgtgtacgcctggaaccgcaagcggatttccaattgtgtcgctgactacagtgtgctttacaactctgcctcattcagtacgttcaagtgttacggcgtgtccccgaccaagctgaatgatctgtgcttcaccaacgtatacgccgactcgtttgtcatccgcggcgatgaagtgaggcagatcgcgcccggccagacgggcaagatcgccgactacaactacaagctgcccgatgatttcactggctgtgttattgcttggaatagtaacaatctggactcgaaggtcggggggaactacaactatctctataggctcttcagaaaaagtaatctgaagcctttcgagcgcgacatctcgactgagatatatcaggcaggctccaccccctgcaacggcgtggaggggtttaattgctacttcccccttcagagctacggctttcagccaacaaacggcgtaggctatcagccctaccgcgtggtggtgctgtcattcgaactcctgcacgcccccgcaaccgtctgcggccctaagaagagtacgaacctggtgaagaacaagtgcgtcaacttcaatttcaacggcctcaccggcacaggggtgctgaccgagtccaacaagaagtttctcccgttccagcagtttgggagggacatcgcagacactaccgacgcggtgagggacccacagaccttggagatactggacatcactccttgcagcttcgggggcgtgtcggtcataactcccggcactaacacctcaaaccaggtcgccgtgctctaccaggacgtgaactgtaccgaggtgcccgtggcgattcacgccgaccagctgacacccacgtggagggtgtactccaccggaagtaatgtcttccagacccgcgccgggtgtctgataggcgccgagcacgtcaacaactcgtacgagggatcccaccatcaccaccaccactga

SEQ ID NO: 4 - protein, 669 aa, Artificial Sequence
  SARS-CoV-2-S1H immunogen
MFVFLVLLPLVSSQCVNLTTRTQLPPAYTNSITRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYEGSHHHHHH

SEQ ID NO: 5 - DNA, 2094 bp, Artificial Sequence
  SARS-CoV-2-S1fRS09 immunogen
atgttcgtgttcctggtcctgctcccactggtgtcctcgcagtgcgtgaacctgaccacccgcacccagctgccccctgcgtacaccaactccatcaccaggggcgtctactatcccgacaaggtgtttcgaagttccgttctgcactcgacccaagacctgttcctccccttcttcagcaacgtgacctggttccatgctatccacgtatcgggtacgaacgggaccaagcgcttcgacaaccccgtcctgcccttcaacgacggcgtgtactttgcatccaccgagaagtcgaacattatcagggggtggattttcggcaccaccctagactcgaagacacagtcgctgctgatcgtgaacaacgcgaccaatgtcgtcatcaaggtttgcgaattccagttctgcaatgaccccttcctgggagtctactaccacaagaacaacaagagctggatggagagtgagttcagagtgtattcctccgctaacaattgcaccttcgagtacgtatcccagcccttcctgatggacctggagggcaagcaggggaactttaaaaatctgcgcgagttcgtgtttaagaacatcgacggctacttcaagatctatagtaagcacacacccataaatctggtccgcgacctgccgcaaggcttctccgctttggaacccctcgtggacctgccgattgggatcaacatcacacgttttcagactctcctggccctgcaccggagttacctgacacccggtgactcctcctcaggctggactgccggggccgccgcttactacgtcggctatttgcagcccagaactttcctcctgaagtataacgagaacggcacgatcaccgatgccgtggattgtgcgctggacccgctgtcggagacaaagtgcacgctgaagtcgtttaccgtggagaaggggatctaccagacatctaacttccgggttcagcccactgagtccatcgtgcgcttccccaacatcacgaatctctgccccttcggcgaggtgttcaacgcaactcgcttcgccagtgtgtacgcctggaaccgcaagcggatttccaattgtgtcgctgactacagtgtgctttacaactctgcctcattcagtacgttcaagtgttacggcgtgtccccgaccaagctgaatgatctgtgcttcaccaacgtatacgccgactcgtttgtcatccgcggcgatgaagtgaggcagatcgcgcccggccagacgggcaagatcgccgactacaactacaagctgcccgatgatttcactggctgtgttattgcttggaatagtaacaatctggactcgaaggtcggggggaactacaactatctctataggctcttcagaaaaagtaatctgaagcctttcgagcgcgacatctcgactgagatatatcaggcaggctccaccccctgcaacggcgtggaggggtttaattgctacttcccccttcagagctacggctttcagccaacaaacggcgtaggctatcagccctaccgcgtggtggtgctgtcattcgaactcctgcacgcccccgcaaccgtctgcggccctaagaagagtacgaacctggtgaagaacaagtgcgtcaacttcaatttcaacggcctcaccggcacaggggtgctgaccgagtccaacaagaagtttctcccgttccagcagtttgggagggacatcgcagacactaccgacgcggtgagggacccacagaccttggagatactggacatcactccttgcagcttcgggggcgtgtcggtcataactcccggcactaacacctcaaaccaggtcgccgtgctctaccaggacgtgaactgtaccgaggtgcccgtggcgattcacgccgaccagctgacacccacgtggagggtgtactccaccggaagtaatgtcttccagacccgcgccgggtgtctgataggcgccgagcacgtcaacaactcgtacgagggatccgggtacatccccgaggcccctcgcgatggccaagcttatgtgcgaaaggacggggagtgggtgctgctctccaccttcctggcccccccccacgcactgagctga

SEQ ID NO: 6 - protein, 697 aa, Artificial Sequence
  SARS-CoV-2-S1fRS09 immunogen
MFVFLVLLPLVSSQCVNLTTRTQLPPAYTNSITRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYEGSGYIPEAPRDGQAYVRKDGEWVLLSTFLAPPHALS

SEQ ID NO: 7 - DNA, 2133 bp, Artificial Sequence
  SARS-CoV-2-S1fRS09H immunogen
atgttcgtgttcctggtcctgctcccactggtgtcctcgcagtgcgtgaacctgaccacccgcacccagctgccccctgcgtacaccaactccatcaccaggggcgtctactatcccgacaaggtgtttcgaagttccgttctgcactcgacccaagacctgttcctccccttcttcagcaacgtgacctggttccatgctatccacgtatcgggtacgaacgggaccaagcgcttcgacaaccccgtcctgcccttcaacgacggcgtgtactttgcatccaccgagaagtcgaacattatcagggggtggattttcggcaccaccctagactcgaagacacagtcgctgctgatcgtgaacaacgcgaccaatgtcgtcatcaaggtttgcgaattccagttctgcaatgaccccttcctgggagtctactaccacaagaacaacaagagctggatggagagtgagttcagagtgtattcctccgctaacaattgcaccttcgagtacgtatcccagcccttcctgatggacctggagggcaagcaggggaactttaaaaatctgcgcgagttcgtgtttaagaacatcgacggctacttcaagatctatagtaagcacacacccataaatctggtccgcgacctgccgcaaggcttctccgctttggaacccctcgtggacctgccgattgggatcaacatcacacgttttcagactctcctggccctgcaccggagttacctgacacccggtgactcctcctcaggctggactgccggggccgccgcttactacgtcggctatttgcagcccagaactttcctcctgaagtataacgagaacggcacgatcaccgatgccgtggattgtgcgctggacccgctgtcggagacaaagtgcacgctgaagtcgtttaccgtggagaaggggatctaccagacatctaacttccgggttcagcccactgagtccatcgtgcgcttccccaacatcacgaatctctgccccttcggcgaggtgttcaacgcaactcgcttcgccagtgtgtacgcctggaaccgcaagcggatttccaattgtgtcgctgactacagtgtgctttacaactctgcctcattcagtacgttcaagtgttacggcgtgtccccgaccaagctgaatgatctgtgcttcaccaacgtatacgccgactcgtttgtcatccgcggcgatgaagtgaggcagatcgcgcccggccagacgggcaagatcgccgactacaactacaagctgcccgatgatttcactggctgtgttattgcttggaatagtaacaatctggactcgaaggtcggggggaactacaactatctctataggctcttcagaaaaagtaatctgaagcctttcgagcgcgacatctcgactgagatatatcaggcaggctccaccccctgcaacggcgtggaggggtttaattgctacttcccccttcagagctacggctttcagccaacaaacggcgtaggctatcagccctaccgcgtggtggtgctgtcattcgaactcctgcacgcccccgcaaccgtctgcggccctaagaagagtacgaacctggtgaagaacaagtgcgtcaacttcaatttcaacggcctcaccggcacaggggtgctgaccgagtccaacaagaagtttctcccgttccagcagtttgggagggacatcgcagacactaccgacgcggtgagggacccacagaccttggagatactggacatcactccttgcagcttcgggggcgtgtcggtcataactcccggcactaacacctcaaaccaggtcgccgtgctctaccaggacgtgaactgtaccgaggtgcccgtggcgattcacgccgaccagctgacacccacgtggagggtgtactccaccggaagtaatgtcttccagacccgcgccgggtgtctgataggcgccgagcacgtcaacaactcgtacgagggatccgggtacatccccgaggcccctcgcgatggccaagcttatgtgcgaaaggacggggagtgggtgctgctctccaccttcctggcccccccccacgcactgagcgagaacttatacttcgagggccaccatcaccaccaccattga

SEQ ID NO: 8 - protein, 710 aa, Artificial Sequence
  SARS-CoV-2-S1fRS09H immunogen
MFVFLVLLPLVSSQCVNLTTRTQLPPAYTNSITRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYEGSGYIPEAPRDGQAYVRKDGEWVLLSTFLAPPHALSENLYFEGHHHHHH

SEQ ID NO: 9 - protein, 663 aa, Artificial Sequence
  SARS-CoV-2-S1 B.1.351 immunogen with C-Tag
MVVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYEPEA

SEQ ID NO: 10 - protein, 664 aa, Artificial Sequence
  SARS-CoV-2-S1 P.1 immunogen with C-Tag
MVVFLVLLPLVSSQCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEYVNNSYEPEA

SEQ ID NO: 11 - protein, 1351 aa, Artificial Sequence
  S1-32AS1-2cg SA SARS-CoV-2-S1 immunogen
MFVFLVLLPLVSSQCVNLTTRTQLPPAYTNSITRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYEGSGQCTNYALLKLAGDVESNPGPVDATMVVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYEPEA

SEQ ID NO: 12 - protein, 1313 aa, Artificial Sequence
  S1-3P2AS1-2cg BZ SARS-CoV-2-S1 immunogen
MFVFLVLLPLVSSQCVNLTTRTQLPPAYTNSITRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYEGSLNSIIKTIQPRVEKKKLDGFYNMMVTNNTFTLKGGAPTKVTFGDDTVIEVQGYKSVNITFELDERIDKVLNEKATYYLFDESGEFKLASHMYCSFYPPDEDEEEGDCEEEEFEPSTQYEPTDNYITTYPGQGLNGYTVEEAKTVLKKCKSAFYILPSIISNEKQELPNAFEYYHTTDPSFLGRYMSALNHTKKWKYPQVNGLTSIKYESLRPDTRYVLMDGSIIQFPNTYLEGSVRVVTELPTGVHAGTDLEGNFYGPFVDRQTALRVESSSKLWAQCVQLHNDILLAKDTEKYNTYKNTCDGTTFTYASALWEIQQVVDADSKIVQLSEISMDNCAAGTTQTACTDDNALAYYNTTKGGRFVLALLSDLQDLKWARFPKSDGTGTIYTELEPPCRFVTDTPKGPKVKYLCLYRNRDVDTDFVNEFYAYLRKHFSMMKVNSTLEQYVFDLSVVNARLRAKHYVYIGDPAQLPAPRTLLTKGTLEPEYRDLYDKLQFTSLEIPRRNVATLQAENVTGLFKDCSKVITGAPFLYLYALVYFLQSINFVRIIMRLWLCWKCRSKNPLLYDANYFLCWHTNCYDYQIGGYTEKWESGVKDCVVLHSYFTSDYYQLYSTQLSTDTIKDLPKEITVATSRTLSYYKLGASQRVAGS

SEQ ID NO: 13 - protein, 1313 aa, Artificial Sequence; identical to SEQ ID NO: 12
  S1-3ORF SARS-CoV-2-S1 immunogen

SEQ ID NO: 14 - protein, 1732 aa, Artificial Sequence
  S1-3ORFN SARS-CoV-2-S1 immunogen
MFVFLVLLPLVSSQCVNLTTRTQLPPAYTNSITRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYEGSLNSIIKTIQPRVEKKKLDGFYNMMVTNNTFTLKGGAPTKVTFGDDTVIEVQGYKSVNITFELDERIDKVLNEKATYYLFDESGEFKLASHMYCSFYPPDEDEEEGDCEEEEFEPSTQYEPTDNYITTYPGQGLNGYTVEEAKTVLKKCKSAFYILPSIISNEKQELPNAFEYYHTTDPSFLGRYMSALNHTKKWKYPQVNGLTSIKYESLRPDTRYVLMDGSIIQFPNTYLEGSVRVVTELPTGVHAGTDLEGNFYGPFVDRQTALRVESSSKLWAQCVQLHNDILLAKDTEKYNTYKNTCDGTTFTYASALWEIQQVVDADSKIVQLSEISMDNCAAGTTQTACTDDNALAYYNTTKGGRFVLALLSDLQDLKWARFPKSDGTGTIYTELEPPCRFVTDTPKGPKVKYLCLYRNRDVDTDFVNEFYAYLRKHFSMMKVNSTLEQYVFDLSVVNARLRAKHYVYIGDPAQLPAPRTLLTKGTLEPEYRDLYDKLQFTSLEIPRRNVATLQAENVTGLFKDCSKVITGAPFLYLYALVYFLQSINFVRIIMRLWLCWKCRSKNPLLYDANYFLCWHTNCYDYQIGGYTEKWESGVKDCVVLHSYFTSDYYQLYSTQLSTDTIKDLPKEITVATSRTLSYYKLGASQRVAGSMSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 15 - protein, 1711 aa, Artificial Sequence
  S1-2SA2ARBDORF SARS-CoV-2-S1 immunogen
MVVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYEGSGSGATNFSLLKQAGDVEENPGPRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGSGATNFSLLKQAGDVEENPGPGDGKMKDLSPRWYFYYLGTGPEAGLPYGANKDGIIWVATEGALNTPKDHIGTRNPGGDAALALLLLDRLNQLESKMSGKGQIAQFAPSASAFFGMSRIGMEVTPSLNKHIDAYKTFPPTEPKKDKKKKADGSLNSIIKTIQPRVEKKKLDGFYNMMVTNNTFTLKGGAPTKVTFGDDTVIEVQGYKSVNITFELDERIDKVLNEKATYYLFDESGEFKLASHMYCSFYPPDEDEEEGDCEEEEFEPSTQYEPTDNYITTYPGQGLNGYTVEEAKTVLKKCKSAFYILPSIISNEKQELPNAFEYYHTTDPSFLGRYMSALNHTKKWKYPQVNGLTSIKYESLRPDTRYVLMDGSIIQFPNTYLEGSVRVVTELPTGVHAGTDLEGNFYGPFVDRQTALRVESSSKLWAQCVQLHNDILLAKDTEKYNTYKNTCDGTTFTYASALWEIQQVVDADSKIVQLSEISMDNCAAGTTQTACTDDNALAYYNTTKGGRFVLALLSDLQDLKWARFPKSDGTGTIYTELEPPCRFVTDTPKGPKVKYLCLYRNRDVDTDFVNEFYAYLRKHFSMMKVNSTLEQYVFDLSVVNARLRAKHYVYIGDPAQLPAPRTLLTKGTLEPEYRDLYDKLQFTSLEIPRRNVATLQAENVTGLFKDCSKVITGAPFLYLYALVYFLQSINFVRIIMRLWLCWKCRSKNPLLYDANYFLCWHTNCYDYQIGGYTEKWESGVKDCVVLHSYFTSDYYQLYSTQLSTDTIKDLPKEITVATSRTLSYYKLGASQRVAGS

SEQ ID NO: 16 - protein, 9 aa, Artificial Sequence
  SARS-CoV-2 CD8+ T cell epitope
KLWAQCVQL

SEQ ID NO: 17 - protein, 9 aa, Artificial Sequence
  SARS-CoV-2 CD8+ T cell epitope
YLQPRTFLL

SEQ ID NO: 18 - protein, 9 aa, Artificial Sequence
  SARS-CoV-2 CD8+ T cell epitope
LLYDANYFL

SEQ ID NO: 19 - protein, 9 aa, Artificial Sequence
  SARS-CoV-2 CD8+ T cell epitope
ALWEIQQVV

SEQ ID NO: 20 - protein, 9 aa, Artificial Sequence
  SARS-CoV-2 CD8+ T cell epitope
LLLDRLNQL

SEQ ID NO: 21 - protein, 11 aa, Artificial Sequence
  SARS-CoV-2 CD8+ T cell epitope
YLFDESGEFKL

SEQ ID NO: 22 - protein, 9 aa, Artificial Sequence
  SARS-CoV-2 CD8+ T cell epitope
FTSDYYQLY

SEQ ID NO: 23 - protein, 10 aa, Artificial Sequence
  SARS-CoV-2 CD8+ T cell epitope
TTDPSFLGRY

SEQ ID NO: 24 - protein, 9 aa, Artificial Sequence
  SARS-CoV-2 CD8+ T cell epitope
PTDNYITTY

SEQ ID NO: 25 - protein, 9 aa, Artificial Sequence
  SARS-CoV-2 CD8+ T cell epitope
ATSRTLSYY

SEQ ID NO: 26 - protein, 10 aa, Artificial Sequence
  SARS-CoV-2 CD8+ T cell epitope
CTDDNALAYY

SEQ ID NO: 27 - protein, 10 aa, Artificial Sequence
  SARS-CoV-2 CD8+ T cell epitope
NTCDGTTFTY

SEQ ID NO: 28 - protein, 9 aa, Artificial Sequence
  SARS-CoV-2 CD8+ T cell epitope
DTDFVNEFY

SEQ ID NO: 29 - protein, 9 aa, Artificial Sequence
  SARS-CoV-2 CD8+ T cell epitope
GTDLEGNFY

SEQ ID NO: 30 - protein, 9 aa, Artificial Sequence
  SARS-CoV-2 CD8+ T cell epitope
KTFPPTEPK

SEQ ID NO: 31 - protein, 9 aa, Artificial Sequence
  SARS-CoV-2 CD8+ T cell epitope
KCYGVSPTK

SEQ ID NO: 32 - protein, 9 aa, Artificial Sequence
  SARS-CoV-2 CD8+ T cell epitope
VTNNTFTLK

SEQ ID NO: 33 - protein, 9 aa, Artificial Sequence
  SARS-CoV-2 CD8+ T cell epitope
KTIQPRVEK

SEQ ID NO: 34 - protein, 9 aa, Artificial Sequence
  SARS-CoV-2 CD8+ T cell epitope
KTFPPTEPK

SEQ ID NO: 35 - protein, 9 aa, Artificial Sequence
  SARS-CoV-2 CD8+ T cell epitope
VTDTPKGPK

SEQ ID NO: 36 - protein, 10 aa, Artificial Sequence
  SARS-CoV-2 CD8+ T cell epitope
ATEGALNTPK

SEQ ID NO: 37 - protein, 9 aa, Artificial Sequence
  SARS-CoV-2 CD8+ T cell epitope
ASAFFGMSR

SEQ ID NO: 38 - protein, 10 aa, Artificial Sequence
  SARS-CoV-2 CD8+ T cell epitope
ATSRTLSYYK

SEQ ID NO: 39 - protein, 9 aa, Artificial Sequence
  SARS-CoV-2 CD8+ T cell epitope
QYIKWPWYI

SEQ ID NO: 40 - protein, 9 aa, Artificial Sequence
  SARS-CoV-2 CD8+ T cell epitope
VYFLQSINF

SEQ ID NO: 41 - protein, 9 aa, Artificial Sequence
  SARS-CoV-2 CD8+ T cell epitope
VYIGDPAQL

SEQ ID NO: 42 - protein, 9 aa, Artificial Sequence
  SARS-CoV-2 CD8+ T cell epitope
SPRWYFYYL

SEQ ID NO: 43 - protein, 8 aa, Artificial Sequence
  SARS-CoV-2 CD8+ T cell epitope
RPDTRYVL

SEQ ID NO: 44 - protein, 9 aa, Artificial Sequence
  SARS-CoV-2 CD8+ T cell epitope
IPRRNVATL

SEQ ID NO: 45 - protein, 648 aa, Artificial Sequence
  ORF1-3-M CD8+ epitope of SARS-CoV-2 ORFab, ORF3, and M-protein genes
LNSIIKTIQPRVEKKKLDGFYNMMVTNNTFTLKGGAPTKVTFGDDTVIEVQGYKSVNITFELDERIDKVLNEKATYYLFDESGEFKLASHMYCSFYPPDEDEEEGDCEEEEFEPSTQYEPTDNYITTYPGQGLNGYTVEEAKTVLKKCKSAFYILPSIISNEKQELPNAFEYYHTTDPSFLGRYMSALNHTKKWKYPQVNGLTSIKYESLRPDTRYVLMDGSIIQFPNTYLEGSVRVVTELPTGVHAGTDLEGNFYGPFVDRQTALRVESSSKLWAQCVQLHNDILLAKDTEKYNTYKNTCDGTTFTYASALWEIQQVVDADSKIVQLSEISMDNCAAGTTQTACTDDNALAYYNTTKGGRFVLALLSDLQDLKWARFPKSDGTGTIYTELEPPCRFVTDTPKGPKVKYLCLYRNRDVDTDFVNEFYAYLRKHFSMMKVNSTLEQYVFDLSVVNARLRAKHYVYIGDPAQLPAPRTLLTKGTLEPEYRDLYDKLQFTSLEIPRRNVATLQAENVTGLFKDCSKVITGAPFLYLYALVYFLQSINFVRIIMRLWLCWKCRSKNPLLYDANYFLCWHTNCYDYQIGGYTEKWESGVKDCVVLHSYFTSDYYQLYSTQLSTDTIKDLPKEITVATSRTLSYYKLGASQRVA

SEQ ID NO: 46 - protein, 780 aa, Artificial Sequence
  N-ORF1-3-M CD8+ epitope of SARS-CoV-2 N, ORFab, ORF3, and M-protein genes
GDGKMKDLSPRWYFYYLGTGPEAGLPYGANKDGIIWVATEGALNTPKDHIGTRNPGGDAALALLLLDRLNQLESKMSGKGQIAQFAPSASAFFGMSRIGMEVTPSLNKHIDAYKTFPPTEPKKDKKKKADRSLNSIIKTIQPRVEKKKLDGFYNMMVTNNTFTLKGGAPTKVTFGDDTVIEVQGYKSVNITFELDERIDKVLNEKATYYLFDESGEFKLASHMYCSFYPPDEDEEEGDCEEEEFEPSTQYEPTDNYITTYPGQGLNGYTVEEAKTVLKKCKSAFYILPSIISNEKQELPNAFEYYHTTDPSFLGRYMSALNHTKKWKYPQVNGLTSIKYESLRPDTRYVLMDGSIIQFPNTYLEGSVRVVTELPTGVHAGTDLEGNFYGPFVDRQTALRVESSSKLWAQCVQLHNDILLAKDTEKYNTYKNTCDGTTFTYASALWEIQQVVDADSKIVQLSEISMDNCAAGTTQTACTDDNALAYYNTTKGGRFVLALLSDLQDLKWARFPKSDGTGTIYTELEPPCRFVTDTPKGPKVKYLCLYRNRDVDTDFVNEFYAYLRKHFSMMKVNSTLEQYVFDLSVVNARLRAKHYVYIGDPAQLPAPRTLLTKGTLEPEYRDLYDKLQFTSLEIPRRNVATLQAENVTGLFKDCSKVITGAPFLYLYALVYFLQSINFVRIIMRLWLCWKCRSKNPLLYDANYFLCWHTNCYDYQIGGYTEKWESGVKDCVVLHSYFTSDYYQLYSTQLSTDTIKDLPKEITVATSRTLSYYKLGASQRVA

SEQ ID NO: 47 - DNA, 1992 bp, Artificial Sequence; identical to SEQ ID NO: 1
  pAd/SARS-CoV-2-S1 immunogen

SEQ ID NO: 48 - DNA, 2133 bp, Artificial Sequence; identical to SEQ ID NO: 7
  pAd/SARS-CoV-2-S1fRS09 immunogen

SEQ ID NO: 49 - DNA, 1992 bp, Artificial Sequence
  pAd/SARS-CoV-2-S1-3 immunogen
atgttcgtgttcttggtgcttctgccgttggtgagttctcagtgcgtcaacctgaccacccgaacccagctcccccccgcctacaccaacagcatcacccggggcgtctactacccagacaaggtgtttcggtctagcgtgctgcactccacccaggacctgttcctccccttcttctccaacgtgacctggttccacgcgatccacgtgtcgggaacgaacggaactaagcgcttcgacaaccctgtgctcccctttaacgatggcgtgtatttcgcttcaaccgagaaatcgaacatcattcgcggttggatcttcggcaccaccctggacagtaagactcagtccctcctgatcgtgaacaatgccacaaacgttgtgatcaaggtctgcgagttccagttttgcaacgaccctttcctgggcgtgtactaccacaagaataacaagtcctggatggagtcagaattcagggtgtacagctcagccaacaactgcacattcgagtatgtgtcccagccctttctgatggatctggagggcaagcaggggaacttcaagaatctacgtgaattcgtgttcaagaacatcgatggctatttcaaaatctactcgaagcacacgcccatcaatctggtaagggacctgcctcaggggttctcggccctggagccactcgtcgatctgccgattggcatcaacatcacccgctttcaaaccctcctggccctccacaggtcctacctgacgcccggcgattcgtcgagtgggtggacggcaggcgcagctgcgtactacgtggggtacctgcagccacgaaccttcctgctgaagtacaacgagaacggcacgattaccgacgcggttgattgcgccttggacccgctctccgaaaccaagtgcaccctgaagagcttcaccgtggagaagggaatctaccagacgagtaacttccgcgtgcagccgaccgagagtattgttcggttccccaacatcaccaacttgtgcccatttggcgaggtcttcaacgccacccgcttcgcaagcgtgtacgcctggaaccgcaagagaatctccaattgcgtggccgactacagcgtcctgtacaactcggcctcgttcagtacgttcaagtgctacggggtgtcccccaccaagctcaatgacctctgctttaccaacgtgtacgctgattcgttcgtaatcaggggtgacgaggtgcgccagatcgccccaggccagactgggaagatcgctgactataactataagctccccgacgactttaccggctgcgtcatcgcctggaactccaacaacctggattcgaaggtgggaggcaactacaattacctgtatcgcctcttcaggaagtcaaatctgaagcctttcgagagggacatatcgaccgagatctaccaggcgggaagtaccccctgcaacggggtggaggggttcaactgctacttcccgctgcagtcgtacggcttccagcctaccaacggggtcgggtaccagccctaccgcgtggtggtgctcagtttcgagctcttgcatgcccccgctacagtgtgcggaccgaagaagtccacaaacctggtgaagaacaagtgcgttaactttaacttcaacggactcactggcaccggcgtgctgactgagtcgaacaagaagtttctgcccttccagcagtttggccgcgacatcgcagacaccaccgatgccgtgcgggacccccagaccctcgagatcctggacatcaccccctgctccttcggcggagtctccgtcataacccccgggacaaacacgtcgaatcaggtggctgtgctctatcaagatgtaaattgtacagaggtgcccgtggcaatccacgcggaccagctgaccccaacctggcgcgtttacagcaccggcagtaacgtgttccagacacgcgctggttgcctcatcggcgccgaacacgtgaacaactcgtatgagggatcctga

SEQ ID NO: 50 - DNA, 3249 bp, Artificial Sequence
  pAd/SARS-CoV-2-S1-3-NP immunogen
atgttcgtgttcttggtgcttctgccgttggtgagttctcagtgcgtcaacctgaccacccgaacccagctcccccccgcctacaccaacagcatcacccggggcgtctactacccagacaaggtgtttcggtctagcgtgctgcactccacccaggacctgttcctccccttcttctccaacgtgacctggttccacgcgatccacgtgtcgggaacgaacggaactaagcgcttcgacaaccctgtgctcccctttaacgatggcgtgtatttcgcttcaaccgagaaatcgaacatcattcgcggttggatcttcggcaccaccctggacagtaagactcagtccctcctgatcgtgaacaatgccacaaacgttgtgatcaaggtctgcgagttccagttttgcaacgaccctttcctgggcgtgtactaccacaagaataacaagtcctggatggagtcagaattcagggtgtacagctcagccaacaactgcacattcgagtatgtgtcccagccctttctgatggatctggagggcaagcaggggaacttcaagaatctacgtgaattcgtgttcaagaacatcgatggctatttcaaaatctactcgaagcacacgcccatcaatctggtaagggacctgcctcaggggttctcggccctggagccactcgtcgatctgccgattggcatcaacatcacccgctttcaaaccctcctggccctccacaggtcctacctgacgcccggcgattcgtcgagtgggtggacggcaggcgcagctgcgtactacgtggggtacctgcagccacgaaccttcctgctgaagtacaacgagaacggcacgattaccgacgcggttgattgcgccttggacccgctctccgaaaccaagtgcaccctgaagagcttcaccgtggagaagggaatctaccagacgagtaacttccgcgtgcagccgaccgagagtattgttcggttccccaacatcaccaacttgtgcccatttggcgaggtcttcaacgccacccgcttcgcaagcgtgtacgcctggaaccgcaagagaatctccaattgcgtggccgactacagcgtcctgtacaactcggcctcgttcagtacgttcaagtgctacggggtgtcccccaccaagctcaatgacctctgctttaccaacgtgtacgctgattcgttcgtaatcaggggtgacgaggtgcgccagatcgccccaggccagactgggaagatcgctgactataactataagctccccgacgactttaccggctgcgtcatcgcctggaactccaacaacctggattcgaaggtgggaggcaactacaattacctgtatcgcctcttcaggaagtcaaatctgaagcctttcgagagggacatatcgaccgagatctaccaggcgggaagtaccccctgcaacggggtggaggggttcaactgctacttcccgctgcagtcgtacggcttccagcctaccaacggggtcgggtaccagccctaccgcgtggtggtgctcagtttcgagctcttgcatgcccccgctacagtgtgcggaccgaagaagtccacaaacctggtgaagaacaagtgcgttaactttaacttcaacggactcactggcaccggcgtgctgactgagtcgaacaagaagtttctgcccttccagcagtttggccgcgacatcgcagacaccaccgatgccgtgcgggacccccagaccctcgagatcctggacatcaccccctgctccttcggcggagtctccgtcataacccccgggacaaacacgtcgaatcaggtggctgtgctctatcaagatgtaaattgtacagaggtgcccgtggcaatccacgcggaccagctgaccccaacctggcgcgtttacagcaccggcagtaacgtgttccagacacgcgctggttgcctcatcggcgccgaacacgtgaacaactcgtatgagggatccatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaa

SEQ ID NO: 51 - DNA, 3324 bp, Artificial Sequence
  pAd/SARS-CoV-2-S1-3-2ANP immunogen
atgttcgtgttcttggtgcttctgccgttggtgagttctcagtgcgtcaacctgaccacccgaacccagctcccccccgcctacaccaacagcatcacccggggcgtctactacccagacaaggtgtttcggtctagcgtgctgcactccacccaggacctgttcctccccttcttctccaacgtgacctggttccacgcgatccacgtgtcgggaacgaacggaactaagcgcttcgacaaccctgtgctcccctttaacgatggcgtgtatttcgcttcaaccgagaaatcgaacatcattcgcggttggatcttcggcaccaccctggacagtaagactcagtccctcctgatcgtgaacaatgccacaaacgttgtgatcaaggtctgcgagttccagttttgcaacgaccctttcctgggcgtgtactaccacaagaataacaagtcctggatggagtcagaattcagggtgtacagctcagccaacaactgcacattcgagtatgtgtcccagccctttctgatggatctggagggcaagcaggggaacttcaagaatctacgtgaattcgtgttcaagaacatcgatggctatttcaaaatctactcgaagcacacgcccatcaatctggtaagggacctgcctcaggggttctcggccctggagccactcgtcgatctgccgattggcatcaacatcacccgctttcaaaccctcctggccctccacaggtcctacctgacgcccggcgattcgtcgagtgggtggacggcaggcgcagctgcgtactacgtggggtacctgcagccacgaaccttcctgctgaagtacaacgagaacggcacgattaccgacgcggttgattgcgccttggacccgctctccgaaaccaagtgcaccctgaagagcttcaccgtggagaagggaatctaccagacgagtaacttccgcgtgcagccgaccgagagtattgttcggttccccaacatcaccaacttgtgcccatttggcgaggtcttcaacgccacccgcttcgcaagcgtgtacgcctggaaccgcaagagaatctccaattgcgtggccgactacagcgtcctgtacaactcggcctcgttcagtacgttcaagtgctacggggtgtcccccaccaagctcaatgacctctgctttaccaacgtgtacgctgattcgttcgtaatcaggggtgacgaggtgcgccagatcgccccaggccagactgggaagatcgctgactataactataagctccccgacgactttaccggctgcgtcatcgcctggaactccaacaacctggattcgaaggtgggaggcaactacaattacctgtatcgcctcttcaggaagtcaaatctgaagcctttcgagagggacatatcgaccgagatctaccaggcgggaagtaccccctgcaacggggtggaggggttcaactgctacttcccgctgcagtcgtacggcttccagcctaccaacggggtcgggtaccagccctaccgcgtggtggtgctcagtttcgagctcttgcatgcccccgctacagtgtgcggaccgaagaagtccacaaacctggtgaagaacaagtgcgttaactttaacttcaacggactcactggcaccggcgtgctgactgagtcgaacaagaagtttctgcccttccagcagtttggccgcgacatcgcagacaccaccgatgccgtgcgggacccccagaccctcgagatcctggacatcaccccctgctccttcggcggagtctccgtcataacccccgggacaaacacgtcgaatcaggtggctgtgctctatcaagatgtaaattgtacagaggtgcccgtggcaatccacgcggaccagctgaccccaacctggcgcgtttacagcaccggcagtaacgtgttccagacacgcgctggttgcctcatcggcgccgaacacgtgaacaactcgtatgagggatccggccagtgtacaaactacgccctgcttaaattagccggcgacgtggagtcaaaccccggccccgtcgacgccaccatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaa

SEQ ID NO: 52 - DNA, 2133 bp, Artificial Sequence
  pAd/SARS-CoV-2-S1-3fRS09 immunogen
atgttcgtgttcttggtgcttctgccgttggtgagttctcagtgcgtcaacctgaccacccgaacccagctcccccccgcctacaccaacagcatcacccggggcgtctactacccagacaaggtgtttcggtctagcgtgctgcactccacccaggacctgttcctccccttcttctccaacgtgacctggttccacgcgatccacgtgtcgggaacgaacggaactaagcgcttcgacaaccctgtgctcccctttaacgatggcgtgtatttcgcttcaaccgagaaatcgaacatcattcgcggttggatcttcggcaccaccctggacagtaagactcagtccctcctgatcgtgaacaatgccacaaacgttgtgatcaaggtctgcgagttccagttttgcaacgaccctttcctgggcgtgtactaccacaagaataacaagtcctggatggagtcagaattcagggtgtacagctcagccaacaactgcacattcgagtatgtgtcccagccctttctgatggatctggagggcaagcaggggaacttcaagaatctacgtgaattcgtgttcaagaacatcgatggctatttcaaaatctactcgaagcacacgcccatcaatctggtaagggacctgcctcaggggttctcggccctggagccactcgtcgatctgccgattggcatcaacatcacccgctttcaaaccctcctggccctccacaggtcctacctgacgcccggcgattcgtcgagtgggtggacggcaggcgcagctgcgtactacgtggggtacctgcagccacgaaccttcctgctgaagtacaacgagaacggcacgattaccgacgcggttgattgcgccttggacccgctctccgaaaccaagtgcaccctgaagagcttcaccgtggagaagggaatctaccagacgagtaacttccgcgtgcagccgaccgagagtattgttcggttccccaacatcaccaacttgtgcccatttggcgaggtcttcaacgccacccgcttcgcaagcgtgtacgcctggaaccgcaagagaatctccaattgcgtggccgactacagcgtcctgtacaactcggcctcgttcagtacgttcaagtgctacggggtgtcccccaccaagctcaatgacctctgctttaccaacgtgtacgctgattcgttcgtaatcaggggtgacgaggtgcgccagatcgccccaggccagactgggaagatcgctgactataactataagctccccgacgactttaccggctgcgtcatcgcctggaactccaacaacctggattcgaaggtgggaggcaactacaattacctgtatcgcctcttcaggaagtcaaatctgaagcctttcgagagggacatatcgaccgagatctaccaggcgggaagtaccccctgcaacggggtggaggggttcaactgctacttcccgctgcagtcgtacggcttccagcctaccaacggggtcgggtaccagccctaccgcgtggtggtgctcagtttcgagctcttgcatgcccccgctacagtgtgcggaccgaagaagtccacaaacctggtgaagaacaagtgcgttaactttaacttcaacggactcactggcaccggcgtgctgactgagtcgaacaagaagtttctgcccttccagcagtttggccgcgacatcgcagacaccaccgatgccgtgcgggacccccagaccctcgagatcctggacatcaccccctgctccttcggcggagtctccgtcataacccccgggacaaacacgtcgaatcaggtggctgtgctctatcaagatgtaaattgtacagaggtgcccgtggcaatccacgcggaccagctgaccccaacctggcgcgtttacagcaccggcagtaacgtgttccagacacgcgctggttgcctcatcggcgccgaacacgtgaacaactcgtatgagggatccgggtacatccccgaggcccctcgcgatggccaagcttatgtgcgaaaggacggggagtgggtgctgctctccaccttcctggcccccccccacgcactgagcgagaacttatacttcgagggccaccatcaccaccaccattga

SEQ ID NO: 53 - DNA, 3357 bp, Artificial Sequence
  pAd/SARS-CoV-2-S1-3fRS09-NP immunogen
atgttcgtgttcttggtgcttctgccgttggtgagttctcagtgcgtcaacctgaccacccgaacccagctcccccccgcctacaccaacagcatcacccggggcgtctactacccagacaaggtgtttcggtctagcgtgctgcactccacccaggacctgttcctccccttcttctccaacgtgacctggttccacgcgatccacgtgtcgggaacgaacggaactaagcgcttcgacaaccctgtgctcccctttaacgatggcgtgtatttcgcttcaaccgagaaatcgaacatcattcgcggttggatcttcggcaccaccctggacagtaagactcagtccctcctgatcgtgaacaatgccacaaacgttgtgatcaaggtctgcgagttccagttttgcaacgaccctttcctgggcgtgtactaccacaagaataacaagtcctggatggagtcagaattcagggtgtacagctcagccaacaactgcacattcgagtatgtgtcccagccctttctgatggatctggagggcaagcaggggaacttcaagaatctacgtgaattcgtgttcaagaacatcgatggctatttcaaaatctactcgaagcacacgcccatcaatctggtaagggacctgcctcaggggttctcggccctggagccactcgtcgatctgccgattggcatcaacatcacccgctttcaaaccctcctggccctccacaggtcctacctgacgcccggcgattcgtcgagtgggtggacggcaggcgcagctgcgtactacgtggggtacctgcagccacgaaccttcctgctgaagtacaacgagaacggcacgattaccgacgcggttgattgcgccttggacccgctctccgaaaccaagtgcaccctgaagagcttcaccgtggagaagggaatctaccagacgagtaacttccgcgtgcagccgaccgagagtattgttcggttccccaacatcaccaacttgtgcccatttggcgaggtcttcaacgccacccgcttcgcaagcgtgtacgcctggaaccgcaagagaatctccaattgcgtggccgactacagcgtcctgtacaactcggcctcgttcagtacgttcaagtgctacggggtgtcccccaccaagctcaatgacctctgctttaccaacgtgtacgctgattcgttcgtaatcaggggtgacgaggtgcgccagatcgccccaggccagactgggaagatcgctgactataactataagctccccgacgactttaccggctgcgtcatcgcctggaactccaacaacctggattcgaaggtgggaggcaactacaattacctgtatcgcctcttcaggaagtcaaatctgaagcctttcgagagggacatatcgaccgagatctaccaggcgggaagtaccccctgcaacggggtggaggggttcaactgctacttcccgctgcagtcgtacggcttccagcctaccaacggggtcgggtaccagccctaccgcgtggtggtgctcagtttcgagctcttgcatgcccccgctacagtgtgcggaccgaagaagtccacaaacctggtgaagaacaagtgcgttaactttaacttcaacggactcactggcaccggcgtgctgactgagtcgaacaagaagtttctgcccttccagcagtttggccgcgacatcgcagacaccaccgatgccgtgcgggacccccagaccctcgagatcctggacatcaccccctgctccttcggcggagtctccgtcataacccccgggacaaacacgtcgaatcaggtggctgtgctctatcaagatgtaaattgtacagaggtgcccgtggcaatccacgcggaccagctgaccccaacctggcgcgtttacagcaccggcagtaacgtgttccagacacgcgctggttgcctcatcggcgccgaacacgtgaacaactcgtatgagggatctgggtacatccccgaggcccctcgcgatggccaagcttatgtgcgaaaggacggggagtgggtgctgctctccaccttcctggcccccccccacgcactgagcggatccatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaa

SEQ ID NO: 54 - DNA, 3432 bp, Artificial Sequence
  pAd/SARS-CoV-2-S1-3fRS09-2ANP immunogen
atgttcgtgttcttggtgcttctgccgttggtgagttctcagtgcgtcaacctgaccacccgaacccagctcccccccgcctacaccaacagcatcacccggggcgtctactacccagacaaggtgtttcggtctagcgtgctgcactccacccaggacctgttcctccccttcttctccaacgtgacctggttccacgcgatccacgtgtcgggaacgaacggaactaagcgcttcgacaaccctgtgctcccctttaacgatggcgtgtatttcgcttcaaccgagaaatcgaacatcattcgcggttggatcttcggcaccaccctggacagtaagactcagtccctcctgatcgtgaacaatgccacaaacgttgtgatcaaggtctgcgagttccagttttgcaacgaccctttcctgggcgtgtactaccacaagaataacaagtcctggatggagtcagaattcagggtgtacagctcagccaacaactgcacattcgagtatgtgtcccagccctttctgatggatctggagggcaagcaggggaacttcaagaatctacgtgaattcgtgttcaagaacatcgatggctatttcaaaatctactcgaagcacacgcccatcaatctggtaagggacctgcctcaggggttctcggccctggagccactcgtcgatctgccgattggcatcaacatcacccgctttcaaaccctcctggccctccacaggtcctacctgacgcccggcgattcgtcgagtgggtggacggcaggcgcagctgcgtactacgtggggtacctgcagccacgaaccttcctgctgaagtacaacgagaacggcacgattaccgacgcggttgattgcgccttggacccgctctccgaaaccaagtgcaccctgaagagcttcaccgtggagaagggaatctaccagacgagtaacttccgcgtgcagccgaccgagagtattgttcggttccccaacatcaccaacttgtgcccatttggcgaggtcttcaacgccacccgcttcgcaagcgtgtacgcctggaaccgcaagagaatctccaattgcgtggccgactacagcgtcctgtacaactcggcctcgttcagtacgttcaagtgctacggggtgtcccccaccaagctcaatgacctctgctttaccaacgtgtacgctgattcgttcgtaatcaggggtgacgaggtgcgccagatcgccccaggccagactgggaagatcgctgactataactataagctccccgacgactttaccggctgcgtcatcgcctggaactccaacaacctggattcgaaggtgggaggcaactacaattacctgtatcgcctcttcaggaagtcaaatctgaagcctttcgagagggacatatcgaccgagatctaccaggcgggaagtaccccctgcaacggggtggaggggttcaactgctacttcccgctgcagtcgtacggcttccagcctaccaacggggtcgggtaccagccctaccgcgtggtggtgctcagtttcgagctcttgcatgcccccgctacagtgtgcggaccgaagaagtccacaaacctggtgaagaacaagtgcgttaactttaacttcaacggactcactggcaccggcgtgctgactgagtcgaacaagaagtttctgcccttccagcagtttggccgcgacatcgcagacaccaccgatgccgtgcgggacccccagaccctcgagatcctggacatcaccccctgctccttcggcggagtctccgtcataacccccgggacaaacacgtcgaatcaggtggctgtgctctatcaagatgtaaattgtacagaggtgcccgtggcaatccacgcggaccagctgaccccaacctggcgcgtttacagcaccggcagtaacgtgttccagacacgcgctggttgcctcatcggcgccgaacacgtgaacaactcgtatgagggatctgggtacatccccgaggcccctcgcgatggccaagcttatgtgcgaaaggacggggagtgggtgctgctctccaccttcctggcccccccccacgcactgagcggatccggccagtgtacaaactacgccctgcttaaattagccggcgacgtggagtcaaaccccggccccgtcgacgccaccatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaa

SEQ ID NO: 55 - protein, 663 aa, Artificial Sequence; identical to SEQ ID NO: 2
  pAd/SARS-CoV-2-S1 immunogen

SEQ ID NO: 56 - protein, 710 aa, Artificial Sequence; identical to SEQ ID NO: 8
  pAd/SARS-CoV-2-S1fRS09 immunogen

SEQ ID NO: 57 - protein, 663 aa, Artificial Sequence; identical to SEQ ID NO: 2
  pAd/SARS-CoV-2-S1-3 immunogen

SEQ ID NO: 58 - protein, 1082 aa, Artificial Sequence
  pAd/SARS-CoV-2-S1-3-NP immunogen
MFVFLVLLPLVSSQCVNLTTRTQLPPAYTNSITRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYEGSMSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 59 - protein, 1107 aa, Artificial Sequence
  pAd/SARS-CoV-2-S1-3-2ANP immunogen
MFVFLVLLPLVSSQCVNLTTRTQLPPAYTNSITRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYEGSGQCTNYALLKLAGDVESNPGPVDATMSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 60 - protein, 710 aa, Artificial Sequence; identical to SEQ ID NO: 8
  pAd/SARS-CoV-2-S1-3fRS09 immunogen

SEQ ID NO: 61 - protein, 1118 aa, Artificial Sequence
  pAd/SARS-CoV-2-S1-3fRS09-NP immunogen
MFVFLVLLPLVSSQCVNLTTRTQLPPAYTNSITRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYEGSGYIPEAPRDGQAYVRKDGEWVLLSTFLAPPHALSGSMSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 62 - protein, 1143 aa, Artificial Sequence
  pAd/SARS-CoV-2-S1-3fRS09-2ANP immunogen
MFVFLVLLPLVSSQCVNLTTRTQLPPAYTNSITRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYEGSGYIPEAPRDGQAYVRKDGEWVLLSTFLAPPHALSGSGQCTNYALLKLAGDVESNPGPVDATMSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 63 - protein, 229 aa, Artificial Sequence
  Immunoglobulin Dimerization Domain
DKTHTCPSRPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 64 - protein, 27 aa, Artificial Sequence
  T4 Fibritin Foldon Trimerization Domain
GYIPEAPRDGQAYVRKDGEWVLLSTFL

SEQ ID NO: 65 - protein, 64 aa, Artificial Sequence
  Human Collagen XV Trimerization Domain
VTAFSNMDDMLQKAHLVIEGTFIYLRDSTEFFIRVRDGWKKLQLGELIPIPADSPPPPALSSNP

(19) United States
(12) Patent Application Publication
(10) Pub. No.: US 2023/0241202 A1
(43) Pub. Date: Aug. 3, 2023

(54) Adenovirus-Based SARS-CoV-2 Vaccine

(71) Applicant: University of Pittsburgh - Of the Commonwealth System of Higher Education, Pittsburgh, PA (US)

(72) Inventors: Andrea A. Gambotto, Pittsburgh, PA (US); Louis Falo, Jr., Wexford, PA (US); Eun Kim, Allison Park, PA (US)

(21) Appl. No.: 17/927,548

(22) PCT Filed: May 28, 2021
(86) PCT No.: PCT/US2021/034874

§ 371 Date: Nov. 23, 2022

Related U.S. Application Data
(60) Provisional application No. 63/031,789, filed on May 29, 2020.

(51) Int. Cl. A61K 39/215; C07K 14/005; C07K 14/195; A61K 39/39; A61P 31/14

(52) U.S. Cl. CPC A61K 39/215; C07K 14/005; C07K 14/195; A61K 39/39; A61P 31/14; C07K 2319/55; A61K 2039/5256

(57) ABSTRACT

A recombinant coronavirus vaccine is provided. Methods of making and delivering the coronavirus vaccine also are provided along with a method of generating and anti-coronavirus immune response. A microneedle array is provided, along with methods of making and using the microneedle array.

### CROSS REFERENCE TO RELATED APPLICATIONS

This application is the United States national phase of International Application No. PCT/US2021/034874, filed May 28, 2021, and claims priority to U.S. Provisional Patent Application No. 63/031,789, filed May 29, 2020, the disclosures of which are hereby incorporated by reference in their entireties.

### STATEMENT REGARDING FEDERAL FUNDING

This invention was made with government support under Grant Nos. AI114264, AR074285, AR071277, AR068248, AI137132, AI043603, and CA175294, awarded by the National Institutes of Health. The government has certain rights in the invention.

Provided herein are coronavirus, e.g., SARS-CoV-2 or SARS-CoV-2, immunogens and vaccines, as well as methods of treating or vaccinating a patient.

On Dec. 31, 2019, Chinese authorities reported a cluster of pneumonia cases in Wuhan, China, most of which included patients who reported exposure to a large seafood market selling many species of live animals. Emergence of another pathogenic zoonotic HCoV was suspected, and by Jan. 10, 2020, researchers from the Shanghai Public Health Clinical Center & School of Public Health and their collaborators released a full genomic sequence of SARS-CoV-2 (related to coronavirus disease 2019, or COVID-19) to public databases, exemplifying prompt data sharing in outbreak response. SARS-CoV-2 uses the angiotensin converting enzyme 2 (ACE2) as a receptor. COVID-19 has reached pandemic status. A number of variants have evolved, such as the South Africa and Brazil variants.

Vaccines and therapeutics able to prevent or mitigate COVID-19, and coronaviruses in general, are sought.

### SUMMARY

Provided herein is a dosage form or pharmaceutical composition, e.g., an intranasal, inhaled, mucosal, or transdermal dosage form or pharmaceutical composition, for reducing, preventing, treating, or vaccinating a patient, comprising a replication-defective adenovirus infectious unit comprising a genome comprising a gene for expressing a polypeptide comprising an immunogenic amino acid sequence of a pathogen (e.g., an epitope or antigen) such as a coronavirus, e.g., SARS-CoV-2, and a pharmaceutically-acceptable excipient.

Also provided herein is a method of eliciting an immune response against a pathogen, such as a coronavirus, e.g., SARS-CoV-2, in a subject, comprising administering to the subject a dosage form or pharmaceutical composition, (e.g., an intranasal, inhaled, mucosal, or transdermal dosage form or pharmaceutical composition), for reducing, preventing, treating, or vaccinating a patient, comprising a replication-defective adenovirus infectious unit comprising a genome comprising a gene for expressing a polypeptide comprising an immunogenic coronavirus, e.g., SARS-CoV-2, amino acid sequence (e.g., an epitope or antigen), and a pharmaceutically-acceptable excipient, thereby eliciting the immune response against the pathogen.

Also provided is a method of preventing infection with SARS-CoV-2 in a patient, comprising vaccinating a patient for SARS-CoV-2, or treating or reducing a symptom of a SARS-CoV-2 infection in a patient, comprising administering to the patient, using a dosage form or pharmaceutical composition, (e.g., an intranasal, inhaled, mucosal, or transdermal dosage form or pharmaceutical composition), for reducing, preventing, treating, or vaccinating a patient, comprising a replication-defective adenovirus infectious unit comprising a genome comprising a gene for expressing a polypeptide comprising an immunogenic amino acid sequence of a SARS-CoV-2 (e.g., an epitope, antigen, antigenic determinant capable of inducing humoral and/or cell-mediated immune response or immunity), and a pharmaceutically-acceptable excipient, thereby eliciting the immune response against the SARS-CoV-2.

Also provided is a nucleic acid, comprising a gene for expressing an immunogenic amino acid sequence as described herein in a replication-defective adenovirus genome.

The following numbered clauses describe various aspects or embodiments of the present invention:

Clause 1. A nucleic acid encoding, or comprising a gene for expressing, a polypeptide comprising, in frame, a coronavirus, such as SARS-CoV-2, S1 protein amino acid sequence and one or both of an epitope string comprising one or more epitopes of a coronavirus, such as SARS-CoV-2, polypeptide; and/or an amino acid sequence having at least 90%, at least 95%, at least 98%, or at least 99% sequence identity with an amino acid sequence of protein of a coronavirus, such as SARS-CoV-2, such as an M protein, an N protein, an ORF1ab protein, an ORF3 protein, an epitope of any of the preceding, or any combination thereof.

Clause 2. The nucleic acid of clause 1, wherein the polypeptide comprises an epitope string comprising one or more epitopes of a coronavirus, such as SARS-CoV-2, polypeptide.

Clause 3. The nucleic acid of clause 2, wherein the epitope string comprises epitopes of a coronavirus, such as SARS-CoV-2, M protein, N protein, ORF1ab protein, and/or ORF3 protein.

Clause 4. The nucleic acid of clause 3, wherein two or more epitopes of the epitope string comprises one or more of the following amino acid sequences: KLWAQCVQL (SEQ ID NO: 16), YLQPRTFLL (SEQ ID NO: 17), LLYDANYFL (SEQ ID NO: 18), ALWEIQQVV (SEQ ID NO: 19), LLLDRLNQL (SEQ ID NO: 20), YLFDESGEFKL (SEQ ID NO: 21), FTSDYYQLY (SEQ ID NO: 22), TTDPSFLGRY (SEQ ID NO: 23), PTDNYITTY (SEQ ID NO: 24), ATSRTLSYY (SEQ ID NO: 25), CTDDNALAYY (SEQ ID NO: 26), NTCDGTTFTY (SEQ ID NO: 27), DTDFVNEFY (SEQ ID NO: 28), GTDLEGNFY (SEQ ID NO: 29), KTFPPTEPK (SEQ ID NO: 30), KCYGVSPTK (SEQ ID NO: 31), VTNNTFTLK (SEQ ID NO: 32), KTIQPRVEK (SEQ ID NO: 33), KTFPPTEPK (SEQ ID NO: 34), VTDTPKGPK (SEQ ID NO: 35), ATEGALNTPK (SEQ ID NO: 36), ASAFFGMSR (SEQ ID NO: 37), ATSRTLSYYK (SEQ ID NO: 38), QYIKWPWYI (SEQ ID NO: 39), VYFLQSINF (SEQ ID NO: 40), VYIGDPAQL (SEQ ID NO: 41), SPRWYFYYL (SEQ ID NO: 42), RPDTRYVL (SEQ ID NO: 43), or IPRRNVATL (SEQ ID NO: 44), separated by spacer amino acids.

Clause 5. The nucleic acid of clause 1, wherein the polypeptide comprises an amino acid sequence having at least 90%, at least 95%, at least 98%, or at least 99% sequence identity with an amino acid sequence of a coronavirus, such as SARS-CoV-2, M protein, N protein, ORF1ab protein, ORF3 protein, an epitope of any of the preceding, or any combination thereof.

Clause 6. The nucleic acid of clause 1, wherein the polypeptide further comprises one or more of: a signal peptide, a self-cleaving sequence, a multimerization sequence, or an affinity tag.

Clause 7. The nucleic acid of clause 1, wherein the polypeptide ranges from 90% to 110% the length of, and has at least 90%, at least 95%, at least 98%, or at least 99% sequence identity with an amino acid sequence of any one of SEQ ID NOS: 11, 12, 13, 14, 15, 58, 59, 60, 61, or 62, or a polypeptide having an amino acid sequence of any one of SEQ ID NOS: 11, 12, 13, 14, 15, 58, 59, 60, 61, or 62.

Clause 8. The nucleic acid of any one of clauses 1-6, wherein the polypeptide further comprises a Toll-like receptor agonist domain, such as a Toll-like receptor 4 (TLR4) or Toll-like receptor 3 (TLR3) agonist domain.

Clause 9. The nucleic acid of clause 8, wherein the Toll-like receptor 4 (TLR-4) agonist domain is an RS09 domain.

Clause 10. The nucleic acid of any one of clauses 1-9, wherein the polypeptide further comprises an angiotensin-converting enzyme 2 (ACE2) receptor-binding domain (RBD) of the SARS-CoV-2 S protein.

Clause 11. The nucleic acid of any one of clauses 1-10 in the form of a mRNA.

Clause 12. The nucleic acid of any one of clauses 1-11, in the form of a gene incorporated into a replication-defective Adenovirus genome, wherein optionally the genome is a gutless Adenovirus genome.

Clause 13. An Adenovirus infectious unit comprising the nucleic acid of clause 10.

Clause 14. A delivery device for delivering to a patient a composition comprising the Adenovirus infectious unit of clause 13.

Clause 15. The device of clause 14, in the form of a nasal sprayer, and inhaler, an aerosol device, or a nebulizer.

Clause 16. The device of clause 14, in the form of a microneedle array comprising a plurality of microneedles.

Clause 17. The device of clause 16, wherein the plurality of microneedles have a shape that comprises a first cross-sectional dimension at a head portion distal to the backing layer, a second cross-sectional dimension at a stem portion proximal to the backing layer, and a third cross-sectional dimension at an intermediate portion located between the top portion and the bottom portion having a cross-sectional dimension greater than the first and second cross-sectional dimensions.

Clause 18. The microneedle array dosage form of clause 16, wherein the plurality of microneedles comprise a stem portion adjacent or proximal to the backing layer, and a head portion attached to the stem portion distal to the backing layer, the microneedles having a barbed or undercut profile in which the head portion having a cross section adjacent to the stem is larger than a cross section of the stem adjacent to the head.

Clause 19. The device of any one of clauses 16-18, wherein the plurality of microneedles comprise a plurality of layers of one or more dissolvable or bioerodible, biocompatible material.

Clause 20. The device of any one of clauses 16-19, wherein a plurality of microneedles comprise carboxymethyl cellulose, trehalose, polyvinylpyrrolidone, maltodextrin, silk, hyaluronic acid, poly(lactic-co-glycolic acid), poly(lactic acid), poly(vinyl alcohol), polyethylene glycol, or a combination of any two or more of the preceding.

Clause 21. The device of any one of clauses 16-20, wherein each of the plurality of microneedles comprises trehalose and comprises a tip portion, wherein the Adenovirus infectious unit is contained in the tip portion.

Clause 22. The device of any one of clauses 16-21, wherein the composition comprises a compound with adjuvant or immune stimulation effect.

Clause 23. The device of clause 22, wherein the compound with adjuvant or immune stimulation effect is: a TLR3 agonist, such as Poly(I:C) or Poly-ICLC; a TLR4 agonist, such as LPS or a monophosphoryl lipid derivative; a TLR5 agonist, such as a flagellin derivative; a TLR 7/8 agonist, such as imiquimod or R848; a TLR 9 agonist, such as CpG sequences; a Stimulator of Interferon Genes (STING) pathway agonist, such as ADU-S100; a stimulatory neuroimmune mediator, such as calcitonin gene-related peptide (CGRP); a neurokinin 1 (NK1) receptor agonist, such as Hemokinin 1 and Substance P; a saponin related adjuvant, such as QS-21 (Quillaja saponaria); a purinoergic receptor agonist, such as ATP; or an oil-in-water emulsion adjuvant, such as MF59.

Clause 24. A method of eliciting an immune response against a coronavirus pathogen in a subject, comprising administering to the subject in a device according to any one of clauses 14-22 a composition comprising the replication-defective adenovirus infectious unit, thereby eliciting the immune response against the pathogen.

Clause 25. The method of clause 24, wherein the pathogen is SARS-CoV-2.

Clause 26. A method of preventing infection with SARS-CoV-2 in a patient, comprising vaccinating a patient for SARS-CoV-2, or treating or reducing a symptom of a SARS-CoV-2 infection in a patient, comprising administering to the patient, using the device of any one of clauses 14-22 a composition comprising the replication-defective adenovirus infectious unit, thereby eliciting the immune response against the SARS-CoV-2.

Clause 27. The method of clause 26, wherein the composition is administered to the patient by inhalation or intra-nasally.

Clause 28. The method of clause 26, wherein the composition is administered to or through the skin, such as intra-cutaneously or subcutaneously, for example by subcutaneous injection of delivery by a microneedle array.

Clause 29. The method of any one of clauses 24-28, wherein the replication-defective adenovirus infectious unit is administered to the patient more than once.

Clause 30. A method of generating an immune response to a coronavirus in a patient comprising administering to a patient a first adenovirus infectious unit comprising a genome comprising a gene for expressing an immunogen polypeptide comprising a coronavirus S1 protein amino acid sequence to produce an immune response to the polypeptide.

Clause 31. The method of clause 30, further comprising administering a second coronavirus immunogen to the patient to produce an immune response to the immunogen in the patient.

Clause 32. The method of clause 31, wherein the second coronavirus immunogen is administered to the patient and an immune response to the immunogen is produced in the patient prior to administration of the first adenovirus infectious unit to the patient.

Clause 33. The method of clause 31, wherein the first adenovirus infectious unit is administered to the patient and an immune response to the polypeptide comprising a coronavirus S1 protein amino acid sequence is produced in the patient prior to administration of the second immunogen to the patient.

Clause 34. The method of any one of clauses 30-33, wherein the second immunogen is produced by a second adenovirus infectious unit comprising a genome comprising a gene for expressing the second immunogen.

Clause 35. The method of clause 34, wherein the gene of the second adenovirus infectious unit for expressing the second immunogen encodes the same polypeptide as the first adenovirus, and the first and second adenovirus infectious units are administered as a prime-boost immunization.

Clause 36. The method of any one of clauses 30-35, wherein the gene for expressing the polypeptide comprising a coronavirus S1 protein amino acid sequence also encodes, in frame with the coronavirus S1 protein amino acid sequence, a coronavirus nucleoprotein amino acid sequence.

Clause 37. The method of any one of clauses 30-36, wherein the gene for expressing the polypeptide comprising a coronavirus S1 protein amino acid sequence also encodes, in frame with the coronavirus S1 protein amino acid sequence, an epitope string comprising one or more epitopes of a coronavirus polypeptide.

Clause 38. The method of any one of clauses 30-36, wherein the gene for expressing the polypeptide comprising a coronavirus S1 protein amino acid sequence also encodes, in frame with the coronavirus S1 protein amino acid sequence, an epitope string of two or more epitopes comprising one or more of the following amino acid sequences: KLWAQCVQL (SEQ ID NO: 16), YLQPRTFLL (SEQ ID NO: 17), LLYDANYFL (SEQ ID NO: 18), ALWEIQQVV (SEQ ID NO: 19), LLLDRLNQL (SEQ ID NO: 20), YLFDESGEFKL (SEQ ID NO: 21), FTSDYYQLY (SEQ ID NO: 22), TTDPSFLGRY (SEQ ID NO: 23), PTDNYITTY (SEQ ID NO: 24), ATSRTLSYY (SEQ ID NO: 25), CTDDNALAYY (SEQ ID NO: 26), NTCDGTTFTY (SEQ ID NO: 27), DTDFVNEFY (SEQ ID NO: 28), GTDLEGNFY (SEQ ID NO: 29), KTFPPTEPK (SEQ ID NO: 30), KCYGVSPTK (SEQ ID NO: 31), VTNNTFTLK (SEQ ID NO: 32), KTIQPRVEK (SEQ ID NO: 33), KTFPPTEPK (SEQ ID NO: 34), VTDTPKGPK (SEQ ID NO: 35), ATEGALNTPK (SEQ ID NO: 36), ASAFFGMSR (SEQ ID NO: 37), ATSRTLSYYK (SEQ ID NO: 38), QYIKWPWYI (SEQ ID NO: 39), VYFLQSINF (SEQ ID NO: 40), VYIGDPAQL (SEQ ID NO: 41), SPRWYFYYL (SEQ ID NO: 42), RPDTRYVL (SEQ ID NO: 43), or IPRRNVATL (SEQ ID NO: 44), separated by spacer amino acids.

Clause 39. The method of any one of clauses 30-36, wherein the gene for expressing the polypeptide encodes a polypeptide ranging from 90% to 110% the length of, and having at least 90%, at least 95%, at least 98%, or at least 99% sequence identity with an amino acid sequence of any one of SEQ ID NOS: 2, 4, 6, 8, 9, 10, 11, 12, 13, 14, 15, 55, 56, 57, 58, 59, 60, 61, or 62.

Clause 40. The method of any one of clauses 30-36, wherein the gene for expressing the polypeptide encodes a polypeptide having an amino acid sequence of any one of SEQ ID NOS: 2, 4, 6, 8, 9, 10, 11, 12, 13, 14, 15, 55, 56, 57, 58, 59, 60, 61, or 62.

Clause 41. The method of any one of clauses 30-36, wherein the coronavirus S1 protein amino acid sequence has an amino acid sequence ranging from 90% to 110% the length of, and having at least 90%, at least 95%, at least 98%, or at least 99% sequence identity with an amino acid sequence of one of SEQ ID NOS: 2, 9, or 10.

Clause 42. The method of any one of clauses 30-36, wherein the gene for expressing the polypeptide comprising a coronavirus S1 protein amino acid sequence also encodes, in frame with the coronavirus S1 protein amino acid sequence: an amino acid sequence having at least 90%, at least 95%, at least 98%, or at least 99% sequence identity with an amino acid sequence of a coronavirus M protein, N protein, ORF1ab protein, ORF3 protein, an epitope of any of the preceding, or any combination thereof.

Clause 43. The method of any one of clauses 30-42, wherein the gene for expressing the polypeptide further comprises, in frame with the coronavirus S1 protein amino acid sequence, one or more of: a signal peptide, a self-cleaving sequence, a multimerization sequence, or an affinity tag.

44. The method of any one of clauses 31-43, wherein the second immunogen comprises:

a coronavirus S1 protein amino acid sequence;
an epitope string comprising one or more epitopes of a coronavirus polypeptide; and/or a polypeptide having an amino acid sequence having at least 90%, at least 95%, at least 98%, or at least 99% sequence identity with an amino acid sequence of a coronavirus M protein, N protein, ORF1ab protein, ORF3 protein, an epitope of any of the preceding, or any combination thereof.

Clause 45. The method of clause 44, wherein the second immunogen comprises an epitope string comprising one or more epitopes of a coronavirus polypeptide.

Clause 46. The method of clause 45, wherein the epitope string comprises epitopes of a coronavirus M protein, N protein, ORF1ab protein, and/or ORF3 protein.

Clause 47. The method of clause 45, wherein one or more epitopes of the epitope string comprises one or more of the following amino acid sequences: KLWAQCVQL (SEQ ID NO: 16), YLQPRTFLL (SEQ ID NO: 17), LLYDANYFL (SEQ ID NO: 18), ALWEIQQVV (SEQ ID NO: 19), LLLDRLNQL (SEQ ID NO: 20), YLFDESGEFKL (SEQ ID NO: 21), FTSDYYQLY (SEQ ID NO: 22), TTDPSFLGRY (SEQ ID NO: 23), PTDNYITTY (SEQ ID NO: 24), ATSRTLSYY (SEQ ID NO: 25), CTDDNALAYY (SEQ ID NO: 26), NTCDGTTFTY (SEQ ID NO: 27), DTDFVNEFY (SEQ ID NO: 28), GTDLEGNFY (SEQ ID NO: 29), KTFPPTEPK (SEQ ID NO: 30), KCYGVSPTK (SEQ ID NO: 31), VTNNTFTLK (SEQ ID NO: 32), KTIQPRVEK (SEQ ID NO: 33), KTFPPTEPK (SEQ ID NO: 34), VTDTPKGPK (SEQ ID NO: 35), ATEGALNTPK (SEQ ID NO: 36), ASAFFGMSR (SEQ ID NO: 37), ATSRTLSYYK (SEQ ID NO: 38), QYIKWPWYI (SEQ ID NO: 39), VYFLQSINF (SEQ ID NO: 40), VYIGDPAQL (SEQ ID NO: 41), SPRWYFYYL (SEQ ID NO: 42), RPDTRYVL (SEQ ID NO: 43), or IPRRNVATL (SEQ ID NO: 44), separated by spacer amino acids.

Clause 48. The method of clause 44, wherein the second immunogen comprises a polypeptide ranging from 90% to 110% the length of, and having at least 90%, at least 95%, at least 98%, or at least 99% sequence identity with an amino acid sequence of any one of SEQ ID NOS: 2, 4, 6, 8, 9, 10, 11, 12, 13, 14, 15, 55, 56, 57, 58, 59, 60, 61, or 62, or a polypeptide having an amino acid sequence of any one of SEQ ID NOS: 2, 4, 6, 8, 9, 10, 11, 12, 13, 14, 15, 55, 56, 57, 58, 59, 60, 61, or 62.

Clause 49 The method of clause 44, wherein the second immunogen comprises an amino acid sequence having at least 90%, at least 95%, at least 98%, or at least 99% sequence identity with an amino acid sequence of a coronavirus M protein, N protein, ORF1ab protein, ORF3 protein, an epitope of any of the preceding, or any combination thereof.

Clause 50. The method of clause 44, wherein the second immunogen comprises one or more of: a signal peptide, a self-cleaving sequence, a multimerization sequence, or an affinity tag.

Clause 51. The method of clause 44, wherein the second immunogen comprises a coronavirus S1 protein amino acid sequence and a coronavirus nucleoprotein amino acid sequence.

Clause 52. The method of any one of clauses 31-51, further comprising administering an adjuvant or immunostimulant with the second immunogen.

Clause 53. The method of any one of clauses 30-52, wherein the coronavirus of any of the coronavirus proteins is SARS-CoV-2.

Clause 54. The method of any one of clauses 30-53, wherein the first adenovirus infectious unit is administered to a patient's mucosa.

Clause 55. The method of clause 54, wherein the first adenovirus infectious unit is administered nasally, optionally as a spray, mist, or aerosol.

Clause 56. The method of any one of clauses 30-55, wherein the second immunogen is the same as the polypeptide produced by the gene of the first adenovirus infectious unit, and is optionally administered using the first adenovirus infectious unit.

Clause 57. The method of any one of clause 30-46, wherein the first adenovirus infectious unit is administered by a different route than the second immunogen.

Clause 58. The method of clause 57, wherein the first adenovirus infectious unit is administered intranasally, and the second immunogen is administered intradermally, optionally using a microneedle array.

Clause 59. The method of clause 57, wherein the second immunogen is administered intramuscularly or dermally (e.g., intradermally and/or subcutaneously), optionally using a microneedle array, and the first adenovirus infectious unit is later administered nasally as a boost.

Clause 60. The method of clause 57, wherein the first adenovirus infectious unit is administered nasally, and the second immunogen is administered intramuscularly or dermally (e.g., intradermally and/or subcutaneously), optionally using a microneedle array, as a boost.

Clause 61. A polypeptide comprising a coronavirus, such as SARS-CoV-2, S1 protein amino acid sequence and one or both of an epitope string comprising one or more epitopes of a coronavirus, such as SARS-CoV-2, polypeptide; and/or an amino acid sequence having at least 90%, at least 95%, at least 98%, or at least 99% sequence identity with an amino acid sequence of protein of a coronavirus, such as SARS-CoV-2, such as an M protein, an N protein, an ORF1ab protein, an ORF3 protein, an epitope of any of the preceding, or any combination thereof.

Clause 62. The polypeptide of clause 61, comprising an epitope string comprising one or more epitopes of a coronavirus, such as SARS-CoV-2, polypeptide.

Clause 63. The polypeptide of clause 62, wherein the epitope string comprises epitopes of a coronavirus, such as SARS-CoV-2, M protein, N protein, ORF1ab protein, and/or ORF3 protein.

Clause 64. The polypeptide of clause 63, wherein two or more epitopes of the epitope string comprises one or more of the following amino acid sequences: KLWAQCVQL (SEQ ID NO: 16), YLQPRTFLL (SEQ ID NO: 17), LLYDANYFL (SEQ ID NO: 18), ALWEIQQVV (SEQ ID NO: 19), LLLDRLNQL (SEQ ID NO: 20), YLFDESGEFKL (SEQ ID NO: 21), FTSDYYQLY (SEQ ID NO: 22), TTDPSFLGRY (SEQ ID NO: 23), PTDNYITTY (SEQ ID NO: 24), ATSRTLSYY (SEQ ID NO: 25), CTDDNALAYY (SEQ ID NO: 26), NTCDGTTFTY (SEQ ID NO: 27), DTDFVNEFY (SEQ ID NO: 28), GTDLEGNFY (SEQ ID NO: 29), KTFPPTEPK (SEQ ID NO: 30), KCYGVSPTK (SEQ ID NO: 31), VTNNTFTLK (SEQ ID NO: 32), KTIQPRVEK (SEQ ID NO: 33), KTFPPTEPK (SEQ ID NO: 34), VTDTPKGPK (SEQ ID NO: 35), ATEGALNTPK (SEQ ID NO: 36), ASAFFGMSR (SEQ ID NO: 37), ATSRTLSYYK (SEQ ID NO: 38), QYIKWPWYI (SEQ ID NO: 39), VYFLQSINF (SEQ ID NO: 40), VYIGDPAQL (SEQ ID NO: 41), SPRWYFYYL (SEQ ID NO: 42), RPDTRYVL (SEQ ID NO: 43), or IPRRNVATL (SEQ ID NO: 44), separated by spacer amino acids.

Clause 65. The polypeptide of clause 61, comprising an amino acid sequence having at least 90%, at least 95%, at least 98%, or at least 99% sequence identity with an amino acid sequence of a coronavirus, such as SARS-CoV-2, M protein, N protein, ORF1ab protein, ORF3 protein, an epitope of any of the preceding, or any combination thereof.

Clause 66. The polypeptide of clause 61, further comprising one or more of: a signal peptide, a self-cleaving sequence, a multimerization sequence, or an affinity tag.

Clause 67. The polypeptide of clause 61, ranging from 90% to 110% the length of, and having at least 90%, at least 95%, at least 98%, or at least 99% sequence identity with an amino acid sequence of any one of SEQ ID NOS: 11, 12, 13, 14, 15, 58, 59, 60, 61, or 62, or having an amino acid sequence of any one of SEQ ID NOS: 11, 12, 13, 14, 15, 58, 59, 60, 61, or 62.

Clause 68. The polypeptide of any one of clauses 61-66, further comprising a Toll-like receptor agonist domain, such as a Toll-like receptor 4 (TLR4) or Toll-like receptor 3 (TLR3) agonist domain.

Clause 69. The polypeptide of clause 68, wherein the Toll-like receptor 4 (TLR-4) agonist domain is an RS09 domain.

Clause 70. The polypeptide of any one of clauses 61-69, further comprising an angiotensin-converting enzyme 2 (ACE2) receptor-binding domain (RBD) of the SARS-CoV-2 S protein.

### BRIEF DESCRIPTION OF THE DRAWINGS

FIGS. 1A-1B: Nucleotide sequence for SARS-CoV-2-S1 (1992 base pairs, SEQ ID NO: 1) (FIG. 1A) and amino acid sequence for SARS-CoV-2-S1 (663 amino acids, SEQ ID NO: 2) (FIG. 1B).

FIGS. 2A-2B: Nucleotide sequence for SARS-CoV-2-S1H (2010 base pairs, SEQ ID NO: 3) (FIG. 2A) and amino acid sequence for SARS-CoV-2-S1H (669 amino acids, SEQ ID NO: 4) (FIG. 2B).

FIGS. 3A-3B: Nucleotide sequence for SARS-CoV-2-S1fRS09 (2094 base pairs, SEQ ID NO: 5) (FIG. 3A) and amino acid sequence for SARS-CoV-2-S1fRS09 (697 amino acids, SEQ ID NO: 6) (FIG. 3B).

FIGS. 4A-4B: Nucleotide sequence for SARS-CoV-2-S1fRS09H (2133 base pairs, SEQ ID NO: 7) (FIG. 4A) and amino acid sequence for SARS-CoV-2-S1fRS09H (710 amino acids, SEQ ID NO: 8) (FIG. 4B).

FIGS. 5A and 5B provide exemplary amino acid sequence of two S1 variants, having a C-terminal affinity tag (-EPEA). FIG. 5A, SEQ ID NO: 9, provides an S1 amino acid sequence for the B.1.351 “South African” variant, including an exemplary adenoviral construct map. Mutations relative to SARS-CoV-2-S1 of FIG. 1B (Wuhan L): De1144; K417N; E484K; N501Y; A570D; and D614G. FIG. 5B, SEQ ID NO: 10, provides an S1 amino acid sequence for the P.1 “Brazil” variant, including an exemplary adenoviral construct map. Mutations relative to SARS-CoV-2-S1 of FIG. 1B (Wuhan L): L18F; T20N; P26S; D138Y; R190S; K417T; E484K; N501Y; and H655Y.

FIGS. 6A-6E provide exemplary amino acid sequences and genome maps for various recombinant adenoviral constructs as described herein, including: FIG. 6A, a construct including a Wuhan S1 sequence in frame with a South African S1 sequence separated by an E2A cleavage sequence (S1-32AS1-2cg SA, SEQ ID NO: 11); FIG. 6B, a construct including a Wuhan S1 sequence in frame with a Brazil S1 sequence separated by an E2A cleavage sequence (S1-32AS1-2cg BZ, SEQ ID NO: 12); FIG. 6C, a construct including a Wuhan S1 sequence in frame with the epitope string of ORF1-3M depicted in FIG. 7B (S1-3ORF, SEQ ID NO: 13); FIG. 6D, a construct including a Wuhan S1 sequence in frame with the epitope string of ORF1-3M, as in FIG. 6C, adding in frame SARS-CoV N-protein sequences (highlighted) (S1-3ORFN, SEQ ID NO: 14); and FIG. 6E, a construct including a South Africa S1 sequence in frame with the Wuhan RBD sequence separated by a P2A cleavage sequence and with the epitope string of N ORF1-3M_depicted in FIG. 7C (S1-3ORFN, SEQ ID NO: 15) separated by a P2A cleavage sequence.

FIG. 7A is a table of shared CD8⁺ T cell epitopes identified in COVID-19 convalescent patients, reproduced from Ferretti A P, et al. Unbiased Screens Show CD8⁺ T Cells of COVID-19 Patients Recognize Shared Epitopes in SARS-CoV-2 that Largely Reside Outside the Spike Protein. Immunity. 2020 Nov. 17; 53(5):1095-1107.e3. doi: 10.1016/j.immuni.2020.10.006. Epub 2020 Oct. 20, Table 1, (SEQ ID NOS: 16-44, respectively). FIGS. 7B-7C depict additional exemplary immunogenic amino acid sequences, including: FIG. 7B a SARS-CoV-2 CD8⁺ epitope string including epitope-containing in-frame portions of the SARS-CoV-2 ORFab, ORF3, and M-protein genes (ORF1-3-M, SEQ ID NO: 45), FIG. 7C a SARS-CoV-2 CD8⁺ epitope string including epitope-containing in-frame portions of the SARS-CoV-2 N, ORFab, ORF3, and M-protein genes (N-ORF1-3-M, SEQ ID NO: 46).

FIGS. 8A and 8B depict schematically exemplary microneedle structures.

FIG. 9. Construction of adenoviral-vectored SARS-CoV-2-S1 vaccine. (A) A shuttle vector carrying the codon-optimized SARS-CoV-2-S1 gene encoding N-terminal 1-661 was designated as shown in the diagram. The vector was used to generate recombinant type 5 replication-deficient adenoviruses by homologous recombination with the adenoviral genomic DNA. ITR: inverted terminal repeat; RBD: receptor binding domain. (B) Detection of the SARS-CoV-2-S1 protein by western blot with the supernatant of A549 cells infected with Ad5.SARS-CoV-2.S1 (Ad5.S1) (10 MOI) using anti spike protein of SARS-CoV rabbit polyclonal antibody (lane2). As a negative control, mock (AdΨ5)-infected cells were treated the same (lane1). The supernatants were resolved on SDS-10% polyacrylamide gel after being boiled in 2% SDS sample buffer with β-ME.

FIG. 10. Induction of humoral immune responses in mice immunized with adenoviral vectored SARS-CoV-2-S1 vaccine. (A) BALB/cJ mice (n=5 per groups) were immunized subcutaneously or intranasally with 1.5×10¹⁰ vp of Ad5.SARS-CoV-2-S1 (Ad5.S1) or AdΨ5, respectively, while mice were immunized subcutaneously with PBS as a negative control. On weeks 0, 2, 4, and 6 after vaccination, the sera from mice were collected, diluted (200×), and tested for the presence of SARS-CoV-2-S1-specific IgG antibody levels at the indicated time points by ELISA. (B) On weeks 0, 2, 4, and 6 after immunization, the sera from mice injected subcutaneously or intranasally with 1.5×10¹⁰ vp of Ad5.S1 were collected, diluted (200×), and tested for the presence of SARS-CoV-2-S1-specific IgM. Significance was determined by one-way ANOVA followed by Tukey's testing (*p<0.05).

FIG. 11. Measurement of two subclasses of SARS-CoV-2-S1-specific IgG antibodies: IgG1 and IgG2a. On weeks 0, 2, 4, and 6 after treatment, sera from mice immunized subcutaneously or intranasally with 1.5×10¹⁰ vp of Ad5.SARS-CoV-2-S1 (Ad5.S1) were collected, diluted (100× for IgG1, 50× for IgG2a), and tested for the presence of SARS-CoV-2-S1-specific IgG1 (A) and IgG2a (B) antibodies by ELISA. Significance was determined by one-way ANOVA followed by Tukey's testing (*p<0.05).

FIG. 12. Neutralizing antibody responses in mice immunized with adenovirus SARS-CoV-2 S1 vaccine. Serum from immunized mice was tested for SARS-CoV-2 neutralizing antibodies using a plaque reduction neutralization test (PRNT). Serum titers that resulted in a 50% reduction in SARS-CoV-2 viral plaques (PRNT50) are reported. Circles and diamonds represent titers from four or six weeks after immunization, respectively, and bars represent means (N=5 mice per group). Significance was determined by Kruskal-Wallis test at each time point, followed by Dunn's multiple comparisons (*p<0.05). The dotted line represents the minimal titer tested (40). Undetectable titers are identified as PRNT50<40. No neutralizing antibodies were detected in serum from 2 weeks (not shown).

FIG. 13. Evaluation of other immune correlates of vaccination-induced protective immunity. (A-B). Analysis of persistent plasma cell responses in the bone marrow of immunized mice. (A) SARS-CoV-2 S1-specific IgG ELISpot analysis of bone marrow cells of BALB/cJ mice 6 weeks after immunization with Ad5.SARS-CoV-2-S1 (Ad5.S1). (B) Qualitative representation of quantitative ELISpot results and plated cell numbers. (C) Quantification of SARS-CoV-2-S1-specific IgA from the lung washes of immunized mice by ELISA. Six weeks 6 after immunization with 1.5×10¹⁰ vp of Ad5.S1 or AdΨ5 subcutaneously or intranasally, bronchoalveolar lavage (BAL) fluid from two mice per group were collected and tested for the presence of SARS-CoV-2-S1-specific IgA.

FIG. 14 provides schematic diagrams of various replication-defective adenovirus constructs (genomes) described herein. S1 is SARS-CoV-2 spike protein, with S1-3 being a codon-optimized version thereof. CMVp is a constitutive CMV promoter. Ψ is the Ad packaging signal, ITR are Ad inverted terminal repeats. NP is SARS-CoV-2 nucleoprotein. RBD is the receptor binding domain. F is a T4 fibritin foldon trimerization domain. Tp is a Tobacco Etch Virus (TEV) protease. 2A is a self-cleaving peptide sequence. RS09 is a Toll-like receptor 4 (TLR-4) agonist peptide. 6H is a His affinity tag. LoxP is the LoxP recombination site.

FIGS. 15A-15H (SEQ ID NOS: 47-54, respectively) provide relevant nucleic acid sequences of the eight constructs shown in FIG. 14.

FIGS. 16A-16H (SEQ ID NOS: 55-62, respectively) provide amino acid sequences of peptides encoded by the eight constructs shown in FIG. 14.

FIG. 17 Neutralizing antibody responses in mice immunized with adenovirus SARS-CoV-2 S1N vaccine by different delivery routes for prime-boost immunization (IN-SC or SC-IN) or by different antigen for prime-boost immunization (Ad.S1N-subunit, rS1) in mice. Serum from immunized mice was tested neutralizing antibodies using a plaque reduction neutralization test (PRNT) with three different SARS-CoV-2 strains from Wuhan (WU COV2), South Africa (SA COV2), or Brazil (BR COV2). Serum titers that resulted in a 90% reduction in SARS-CoV-2 viral plaques (NT₉₀) compared to the virus control are reported at six, eight, and nine weeks after immunization, respectively, and bars represent means (N=5 mice per group). No neutralizing antibodies were detected in serum PBS control group (not shown).

FIG. 18 Antigen-specific antibody responses in mice immunized with recombinant S1 (rS1) proteins and adenoviral vectored SARS-CoV-2-S1N vaccines. BALB/c mice (n=5 per groups) were immunized intramuscularly with 15 ug of Wuhan rS1 (S1-2cg WU), South Africa rS1 (S1 SA), mixture of Wuhan rS1 and South Africa rS1 (S1-2cg WU+S1 SA), S1-FRS09, or 1×10¹⁰ vp of Ad5.SARS-CoV-2-S1N (Ad5.S1N), while mice were immunized intramuscularly with PBS as a negative control. At three weeks after prime injection, mice were boosted with homologous or heterologous (S1-2cg WU-S1 SA, Ad5.S1N-S1-2cg WU, Ad5.S1N-S1-S1 SA), respectively, On weeks 0, 3, 5, and 7 after vaccination, the sera from mice were collected, diluted (200×), and tested for the presence of SARS-CoV-2-S1-specific IgG antibody levels at the indicated time points by ELISA. Horizontal lines represent geometric mean antibody titers. Significance was determined by Kruskal-Wallis test followed by Dunn's multiple comparisons (*p<0.05).

### DETAILED DESCRIPTION

Provided herein are adenovirus vector-based monomeric and multimeric SARS-CoV-2 vaccines for delivery of immunogens for eliciting a neutralizing immune response against a coronavirus, such as SARS-CoV-2, including variants thereof. Immunogens may comprise complete or partial sequences of coronavirus proteins, including the S1, N, M, ORF1ab, and ORF3 proteins, including epitope strings comprising epitopes derived from any single, combination, or all of the coronavirus S1, N, M, ORF1ab, and ORF3 proteins. Additional amino acids sequences, such as a T4 fibritin foldon trimerization domain, a Toll-like receptor 4 (TLR-4) agonist peptide (RS09), and/or a self-cleaving peptide, may be included in-frame in the coding sequence to best elicit an immune response. To optimize production of an effective immune response, a heterologous prime-boost strategy may be utilized, in which different immunogens are presented to a patient at different times, and/or different routes of immunization are used for the prime and boost stages of the immunization.

Other than in the operating examples, or where otherwise indicated, the use of numerical values in the various ranges specified in this application are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of ranges is intended as a continuous range including every value between the minimum and maximum values. Further, as used herein, all numbers expressing dimensions, physical characteristics, processing parameters, quantities of ingredients, reaction conditions, and the like, used in the specification and claims are to be understood as being modified in all instances by the term “about”. Moreover, unless otherwise specified, all ranges disclosed herein are to be understood to encompass the beginning and ending range values and any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more and ending with a maximum value of 10 or less, e.g., 1 to 3.3, 4.7 to 7.5, 5.5 to 10, and the like.

As used herein “a” and “an” refer to one or more. The term “comprising” is open-ended and may be synonymous with “including”, “containing”, or “characterized by”. The term “consisting essentially of” limits the scope of a claim to the specified materials or steps and those that do not materially affect the basic and novel characteristic(s) of the claimed invention.

As used herein, spatial or directional terms, such as “left”, “right”, “inner”, “outer”, “above”, “below”, “over”, “under”, and the like, relate to the invention as it is shown in the drawing figures are provided solely for ease of description and illustration, and do not imply directionality, unless specifically required for operation of the described aspect of the invention. It is to be understood that the invention can assume various alternative orientations and, accordingly, such terms are not to be considered as limiting.

As used herein, a “patient” or “subject” is an animal, such as a mammal, including a primate (such as a human, a non-human primate, e.g., a monkey, and a chimpanzee), a non-primate (such as a cow, a pig, a camel, a llama, a horse, a goat, a rabbit, a sheep, a hamster, a guinea pig, a cat, a dog, a rat, a mouse, a horse, and a whale), or a bird (e.g., a duck or a goose). As used herein, the terms “treating”, or “treatment” refer to a beneficial or desired result, such as improving one of more functions, or symptoms of a disease.

Unless stated otherwise, nucleotide sequences are recited herein in a 5′ to 3′ direction, and amino acid sequences are recited herein in an N-terminal to C-terminal direction according to convention.

“Therapeutically effective amount,” as used herein, is intended to include the amount of a therapeutic agent, such as an immunogen, as described herein that, when administered to a subject having a disease, is sufficient to effect treatment of the disease (e.g., by diminishing, ameliorating or maintaining the existing disease or one or more symptoms of disease). The “therapeutically effective amount” may vary depending on compound or composition, how it is administered, the disease and its severity and the history, age, weight, family history, genetic makeup, the types of preceding or concomitant treatments, if any, and other individual characteristics of the subject to be treated.

A “therapeutically-effective amount” also includes an amount of an agent that produces some desired local or systemic effect at a reasonable benefit/risk ratio applicable to any treatment. Compounds and compositions described herein may be administered in a sufficient amount to produce a reasonable benefit/risk ratio applicable to such treatment. For example, a therapeutically-effective amount of a virus vaccine useful for eliciting an immune response in a subject and/or for preventing infection by the virus. In the context of the present disclosure, a therapeutically effective amount of a coronavirus, e.g., a SARS-CoV-2, virus vaccine, for example, is an amount sufficient to increase resistance to, prevent, ameliorate, and/or treat infection caused by a coronavirus, e.g., a SARS-CoV-2 virus in a subject without causing a substantial cytotoxic effect in the subject. The effective amount of a coronavirus, e.g., a SARS-CoV-2, virus vaccine or immunogen useful for increasing resistance to, preventing, ameliorating, and/or treating infection in a subject will be dependent on, for example, the subject being treated, the manner of administration of the therapeutic composition and other factors.

A non-limiting range for a therapeutically effective amount of the disclosed immunogen within the methods and immunogenic compositions of the disclosure is about 0.0001 mg/kg body weight to about 10 mg/kg body weight, such as about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.5 mg/kg, about 2 mg/kg, about 2.5 mg/kg, about 3 mg/kg, about 4 mg/kg, about 5 mg/kg, or about 10 mg/kg, for example, 0.01 mg/kg to about 1 mg/kg body weight, about 0.05 mg/kg to about 5 mg/kg body weight, about 0.2 mg/kg to about 2 mg/kg body weight, or about 1.0 mg/kg to about 10 mg/kg body weight. In some embodiments, the dosage includes a set amount of a disclosed immunogen such as from about 1-300 μg, for example, a dosage of about 1, 2, 3, 4, 5, 6, 7, 8, 9, 10, 15, 20, 30, 40, 50, 60, 70, 80, 90, 100, 150, 200, 250, or about 300 μg.

Dosage can be varied by the attending clinician to maintain a desired concentration at a target site (for example, systemic circulation). Higher or lower concentrations can be selected based on the mode of delivery, for example, trans-epidermal, rectal, oral, pulmonary, or intranasal delivery versus intravenous or subcutaneous delivery. The actual dosage of disclosed immunogen will vary according to factors such as the disease indication and particular status of the subject (for example, the subject's age, size, fitness, extent of symptoms, susceptibility factors, and the like), time and route of administration, other drugs or treatments being administered concurrently, as well as the specific pharmacology of the composition for eliciting the desired activity or biological response in the subject. Dosage regimens can be adjusted to provide an optimum prophylactic or therapeutic response. A therapeutically effective amount is also one in which any toxic or detrimental side effects of the disclosed immunogen and/or other biologically active agent is outweighed in clinical terms by therapeutically beneficial effects.

An immunogen is an antigen able to induce a humoral and/or a cell-mediated immune response. A composition comprising an immunogen includes additional carriers or excipients, including, for example, and without limitation:
Lipid(s), as with liposomes, micellar structures, emulsions, and lipid nanoparticles comprising the immunogen or a nucleic acid, such as mRNA or DNA, encoding a polypeptide immunogen;
Water, salt(s), buffer(s), and/or rheology modifier(s); and adjuvant(s).

Further, preventing, treating or ameliorating a disease: “Preventing” a disease refers to inhibiting the full development of a disease or infection, such as a coronavirus, e.g. a SARS-Cov-2 infection, from a subsequent exposure. “Treating” refers to a therapeutic intervention that ameliorates a sign or symptom of a disease or pathological condition after it has begun to develop. “Ameliorating” refers to the reduction in the number or severity of one or more signs or symptoms of a disease or infection. A prime-boost vaccination refers to an immunotherapy including administration of a first immunogenic composition (the primer vaccine) followed by administration of a second immunogenic composition (the booster vaccine) to a subject to elicit an immune response. The primer vaccine and/or the booster vaccine may include a vector (such as a viral vector, RNA, or DNA vector) expressing the antigen to which the immune response is directed, or can include a protein immunogen. The booster vaccine is administered to the subject after the primer vaccine; the skilled artisan will understand a suitable time interval between administration of the primer vaccine and the booster vaccine, and examples of such timeframes are disclosed herein. In some embodiments, the primer vaccine, the booster vaccine, or both primer vaccine and the booster vaccine additionally include an adjuvant.

The prime and boost immunizations may deliver the same immunogen or sequence encoding the immunogen by the same route to the patient, as is commonly performed.

The prime immunization may deliver the immunogen or a sequence encoding the immunogen, by a first route to the patient, and the boost immunization may deliver the same immunogen or sequence encoding the immunogen by a different route to the patient. A first immunization may be delivered intramuscularly, with the boost immunization delivered mucosally, intranasally, nasopharyngeally, or intradermally. Alternatively, first immunization may be delivered mucosally, intranasally, nasopharyngeally, or intradermally, with the boost immunization delivered intramuscularly. The immunization may be via an adenoviral vector as described herein, for example when the route of delivery is to a patient's mucosa, as in nasal or nasopharyngeal delivery. Delivery intramuscularly, intradermally, or subdermally may utilize an mRNA-lipid nanoparticle vaccine, or a purified protein, optionally with adjuvant vaccine. Of note, any protein described herein may be delivered either as a purified protein, optionally with adjuvant, as an mRNA, or via a viral vector, such as the Ad vectors described herein.

The prime immunization may deliver or encode a first immunogen, and the boost immunization may deliver or encode a different immunogen. The immunogen of the first boost or further boosts may be a mutated variant of an antigen presented in the prime immunization, such as a Wuhan S1 protein being provided in the first immunization, and the South Africa and/or Brazil S1 variant being provided in a boost immunization. The immunogen of the boost and/or further boosts may provide different antigens, such as different viral proteins or epitope strings, and different epitopes or proteins for eliciting different types of immune responses, as in providing amino acid sequences comprising CD8⁺ epitopes. Different immunogens or sequences encoding immunogens may be delivered by different routes in the prime and boost stage(s). In one example, in a patient previously immunized using an intramuscular-delivered mRNA-lipid nanoparticle for producing an S1 protein, the patient is then “boosted” with an immunogen as described herein by the same route or a different route, such as using an adenoviral vector to deliver sequences encoding an immunogen, e.g., as described herein, mucosally or intradermally.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the subject being treated. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) lubricating agents, such as magnesium state, sodium lauryl sulfate, and talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates, and/or polyanhydrides; (22) bulking agents, such as polypeptides and amino acids (23) serum component, such as serum albumin, HDL and LDL; and (22) other non-toxic compatible substances employed in pharmaceutical formulations. Remington: The Science and Practice of Pharmacy, The University of the Sciences in Philadelphia, Editor, Lippincott, Williams, & Wilkins, Philadelphia, Pa., 21st Edition (2005), describes compositions and formulations suitable for pharmaceutical delivery of one or more therapeutic compositions, such as a chimeric virus, and additional pharmaceutical agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Therapeutic agents, for example and without limitation, bioactive agents, drugs, active pharmaceutical ingredients, or biologicals, may be incorporated into the compositions and combination devices described herein. Non-limiting examples of therapeutic agents include: anti-inflammatories, such as, without limitation, NSAIDs (non-steroidal anti-inflammatory drugs) such as salicylic acid, indomethacin, sodium indomethacin trihydrate, salicylamide, naproxen, colchicine, fenoprofen, sulindac, diflunisal, diclofenac, indoprofen sodium salicylamide, anti-inflammatory cytokines, and anti-inflammatory proteins or steroidal anti-inflammatory agents); antibiotics; anticlotting factors such as heparin, Pebac, enoxaprin, aspirin, hirudin, plavix, bivalirudin, prasugrel, idraparinux, warfarin, coumadin, clopidogrel, PPACK, GGACK, tissue plasminogen activator, urokinase, and streptokinase; growth factors; or antibiotics, such as, without limitation: acyclovir, afloxacin, ampicillin, amphotericin B, atovaquone, azithromycin, ciprofloxacin, clarithromycin, clindamycin, clofazimine, dapsone, diclazaril, doxycycline, erythromycin, ethambutol, fluconazole, fluoroquinolones, foscarnet, ganciclovir, gentamicin, iatroconazole, isoniazid, ketoconazole, levofloxacin, lincomycin, miconazole, neomycin, norfloxacin, ofloxacin, paromomycin, penicillin, pentamidine, polymixin B, pyrazinamide, pyrimethamine, rifabutin, rifampin, sparfloxacin, streptomycin, sulfadiazine, tetracycline, tobramycin, trifluorouridine, trimethoprim sulphate, Zn-pyrithione, ciprofloxacin, norfloxacin, afloxacin, levofloxacin, gentamicin, tobramycin, neomycin, erythromycin, trimethoprim sulphate, polymixin B, and silver salts such as chloride, bromide, iodide and periodate; cytokines or chemoattractants; an anti-inflammatory protein; a steroidal anti-inflammatory agent; or an anticlotting agents, such as heparin. Other therapeutic agents that may promote immunogenicity of the described immunogen may also be included.

As used herein, administering a composition (e.g., an immunogenic composition, such as a vaccine) to a subject means to give, apply or bring the composition into contact with the subject. Administration can be accomplished by any of a number of routes, such as, for example, topical, oral, subcutaneous, intradermal intramuscular, transdermal, mucosal, intraperitoneal, intravenous, intrathecal, and intramuscular.

Transdermal drug delivery methods use various methods to permeate the stratum corneum, and include microneedle or microneedle array, thermal ablation, microdermabrasion, electroporation, and cavitational ultrasound methods and related dosage forms for delivery of therapeutic agents through the stratum corneum. Certain transdermal delivery methods may be preferred for delivery of larger virus particles, such as dermabrasion and microneedle/microneedle array delivery methods (See, e.g., Prausnitz, M. R., et al., Transdermal Drug Delivery Nat Biotechnol. 2008 November; 26(11): 1261-1268. doi:10.1038/nbt.1504 and Alkilani, A. Z., et al., Transdermal Drug Delivery: Innovative Pharmaceutical Developments Based on Disruption of the Barrier Properties of the stratum corneum. Pharmaceutics 2015, 7, 438-470; doi:10.3390/pharmaceutics7040438).

Mucosal drug delivery systems (e.g., transmucosal delivery) include delivery through the pulmonary, oral, nasal, ocular, vaginal, and rectal routes, and include, without limitation: oral mucoadhesive/superdisintegrating tablets, bi-therapy tablets, mucoadhesive films, patches or wafers, sublingual capsules, gels and sprays; wet or dry powder nasal sprays, foams, aerosols, patches, nano-carrier loaded films, gels and hydrogels, and drops; ocular drops, gels, solutions, patches, and films; vaginal pessaries, rings, gels sprays, and foams; rectal suppositories, capsules, tablets, gels, films, and multilayered patches; and pulmonary aerosols, sprays, and dry powder inhalers (See, e.g., Goyal, A. K., et al., (2018) Noninvasive systemic drug delivery through mucosal routes, Artificial Cells, Nanomedicine, and Biotechnology, 46:sup2, 539-551, DOI: 10.1080/21691401.2018.1463230).

An antibody is an immunoglobulin molecule produced by B lymphoid cells with a specific amino acid sequence. Antibodies are evoked in humans or other animals by a specific antigen (immunogen). Antibodies are characterized by reacting specifically with the antigen in some demonstrable way, antibody and antigen each being defined in terms of the other. “Eliciting an antibody response” refers to the ability of an antigen or other molecule to induce the production of antibodies.

An immunogen is a compound, composition, or substance that can stimulate the production of antibodies or a T-cell response in an animal, including compositions that are injected or absorbed into an animal. An antigen reacts with the products of specific humoral or cellular immunity, including those induced by heterologous immunogens. In one embodiment, an immunogen is a virus antigen, such as a coronavirus spike protein.

A biological sample is a sample obtained from a subject (such as a human or veterinary subject). Biological samples, include, for example, fluid, cell and/or tissue samples. In some embodiments herein, the biological sample is a fluid sample. Fluid sample include, but are not limited to, serum, blood, plasma, urine, feces, saliva, cerebral spinal fluid (CSF), and bronchoalveolar lavage (BAL) fluid.

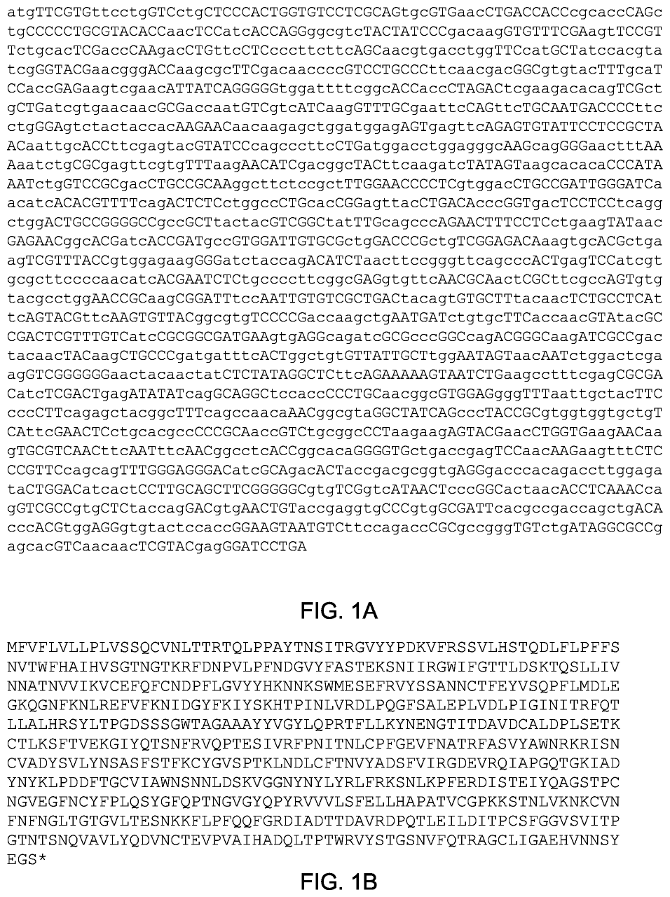

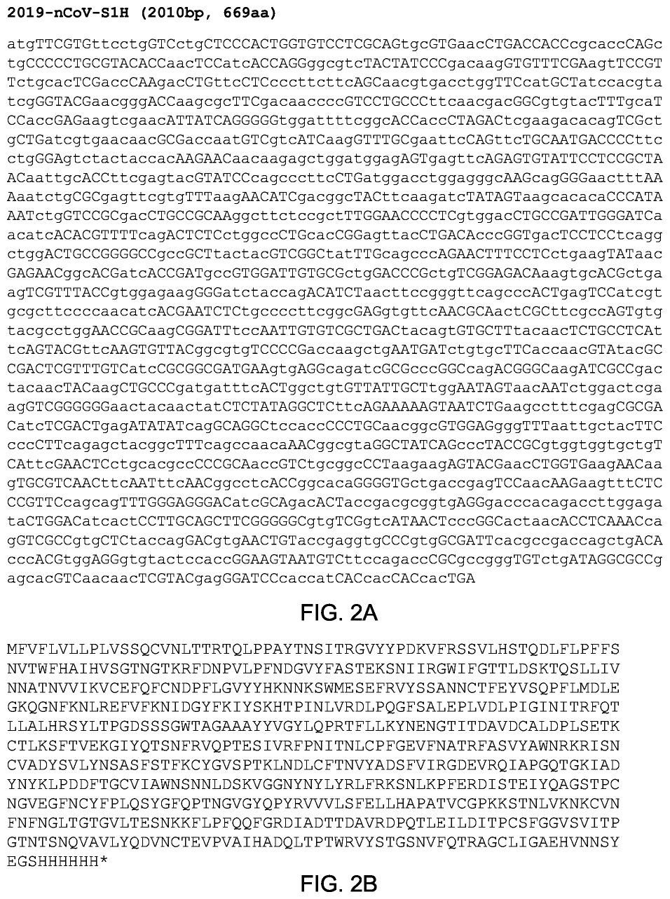

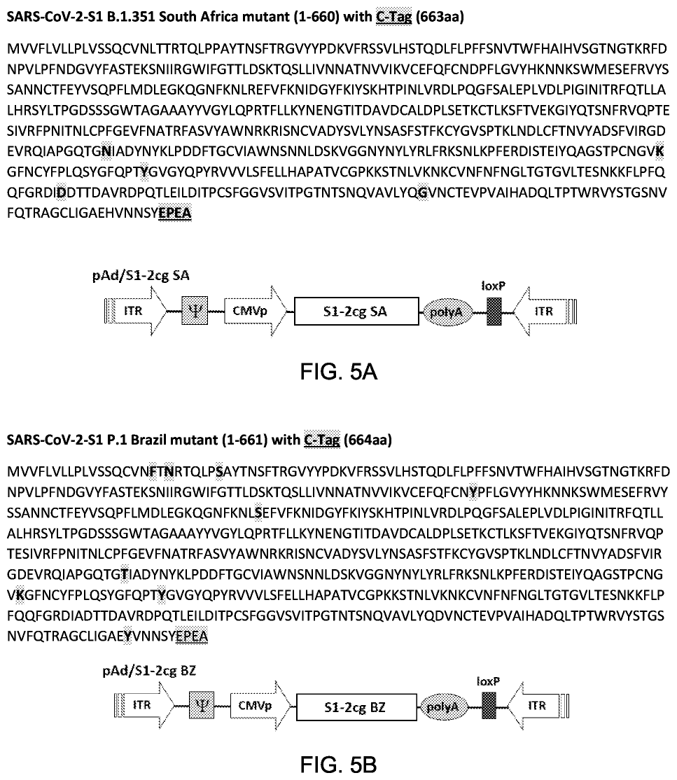

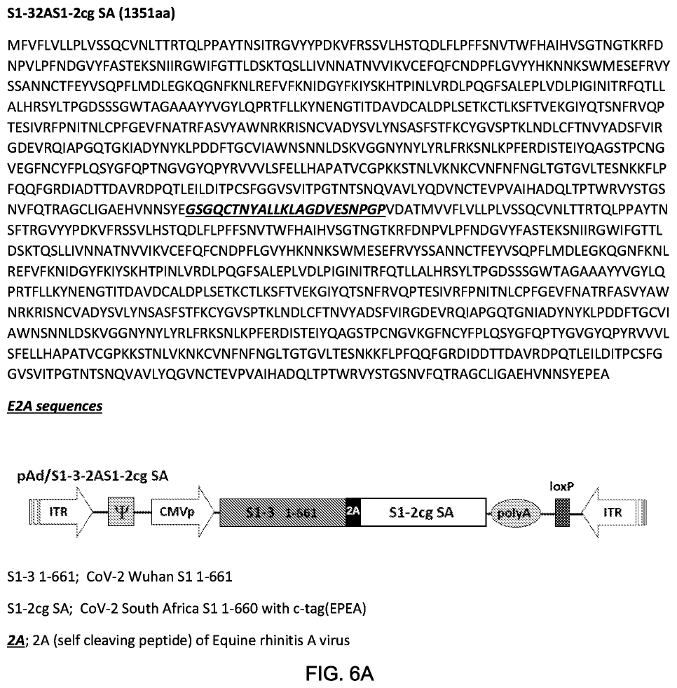

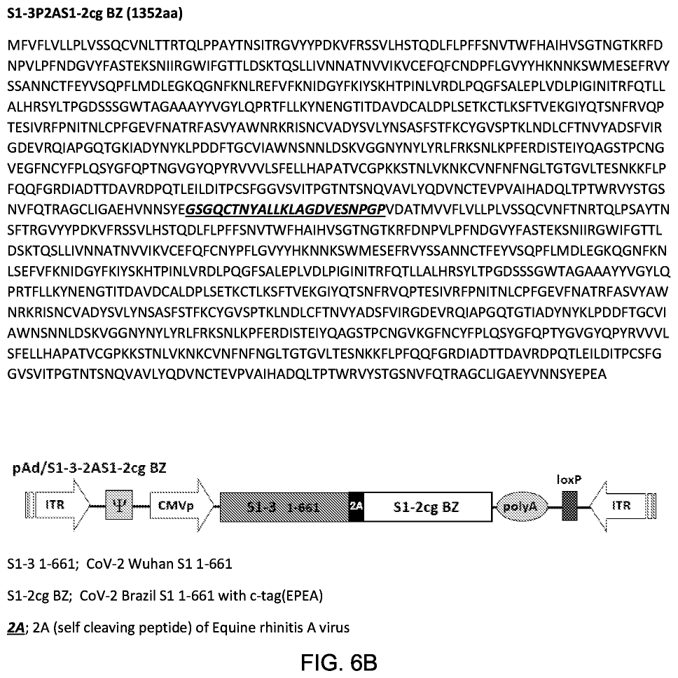

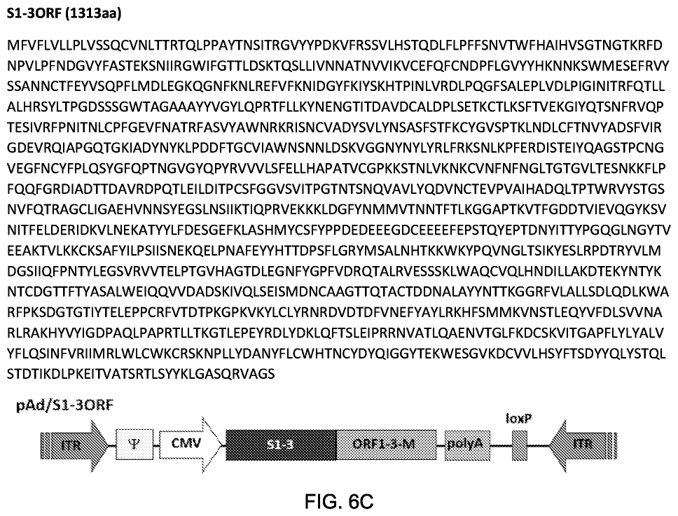

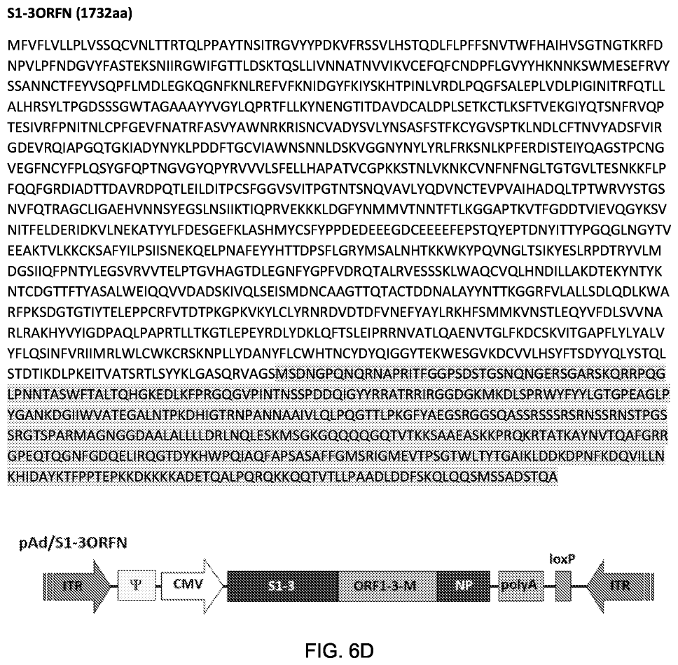

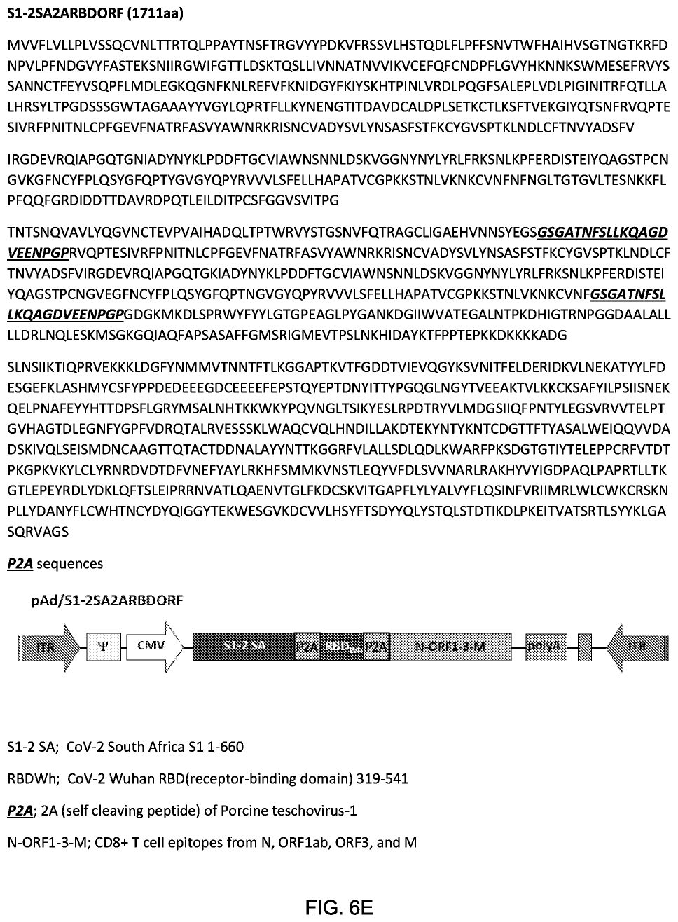

Coronaviruses (Coronoviridae) are members of the Nidovirales order, and are enveloped, non-segmented positive-sense RNA viruses. The most prominent feature of coronaviruses is the club-shape spike projections emanating from the surface of the virion. These spikes are a defining feature of the virion and give them the appearance of a solar corona, prompting the name, coronaviruses. Homotrimers of the virus encoded S protein make up the distinctive spike structure on the surface of the virus. The trimeric S glycoprotein is a class I fusion protein and mediates attachment to the host receptor. In most, but not all, coronaviruses, S is cleaved by a host cell furin-like protease into two separate polypeptides noted S1 and S2. S1 makes up the large receptor-binding domain of the S protein while S2 forms the stalk of the spike. Examples of sequences of the SARS-CoV-2 spike protein are provided in FIGS. 1A, 1B, 2A, 2B, 3A, 3B, 4A, 4B, 5A, and 5B. SARS-CoV, MERS-CoV, and SARS-CoV-2 are members of the betacoronavirus (or group 2 coronavirus) genera. Coronavirus proteins include, for example and without limitation, and among others: spike (S1 and S2) proteins, M (membrane) protein, N (nucleocapsid) protein, ORF1ab (ORF1a and ORF1b) proteins, ORF3 proteins, the sequences of which are broadly published and are freely available (See, e.g., NCBI Reference Sequence: NC_045512.2, Severe acute respiratory syndrome coronavirus 2 isolate Wuhan-Hu-1, complete genome, providing nucleotide and protein sequences). Immunogens may comprise any full-length or partial sequences of any coronavirus protein, or epitope(s) thereof.

A “codon-optimized” nucleic acid refers to a nucleic acid sequence that has been altered such that the codons are optimal for expression in a particular system (such as a particular species of group of species). For example, a nucleic acid sequence can be optimized for expression in mammalian cells. Codon optimization does not alter the amino acid sequence of the encoded protein.

A conservative substitution is a substitution of one amino acid residue in a protein sequence for a different amino acid residue having similar biochemical properties. Typically, conservative substitutions have little to no impact on the activity of a resulting polypeptide. For example, ideally, a coronavirus protein (such as an S protein) including one or more conservative substitutions (for example 1-10, 2-5, or 10-20, or no more than 2, 5, 10, 20, 30, 40, or 50 substitutions) retains the structure and function of the wild-type protein, namely, in the context of the present disclosure, the ability to elicit an immunological response, such as a neutralizing immunological response to SARS-CoV-2, e.g., the spike protein of SARS-CoV-2. A polypeptide can be produced to contain one or more conservative substitutions by manipulating the nucleotide sequence that encodes that polypeptide using, for example, standard procedures such as site-directed mutagenesis or PCR. In one example, such variants can be readily selected for additional testing by infecting cells with a virus containing a variant protein and determining its ability to replicate, by producing virus containing a variant protein and determining its virulence or cell-invasion properties, and/or by testing antibody cross-reactivity.

The term “contacting” refers to placement in direct physical association; includes both in solid and liquid form. “Contacting” is often used interchangeably with “exposed.” In some cases, “contacting” includes transfecting, such as transfecting a nucleic acid molecule into a cell. In other examples, “contacting” refers to incubating a molecule (such as an antibody) with a biological sample.

A fusion protein or fusion polypeptide refers to a protein or polypeptide generated, for example, by expression of a nucleic acid sequence engineered from nucleic acid sequences encoding at least a portion of two different (heterologous) proteins. To create a fusion protein, the nucleic acid sequences are in the same reading frame and contain no internal stop codons. For example, a fusion protein includes a coronavirus S or S1 polypeptide fused to a heterologous protein, that is an amino acid sequence originating synthetically, or from a different genetic source or species.

An epitope is an antigenic determinant capable of inducing humoral and/or cell-mediated immune response or immunity, that is the portion of an antigen that is recognized by the immune system, and in the case of a protein antigen, comprises a specific amino acid sequence. Epitopes may be identified by any useful epitope mapping method, e.g., as are broadly-known in the arts.

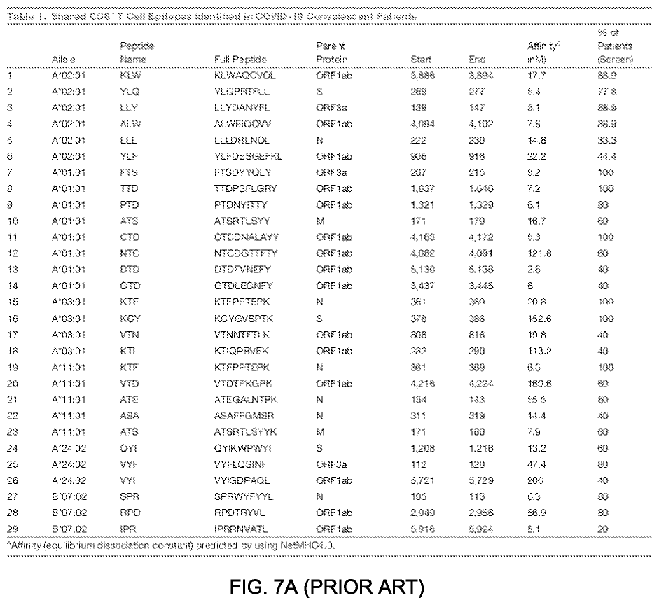

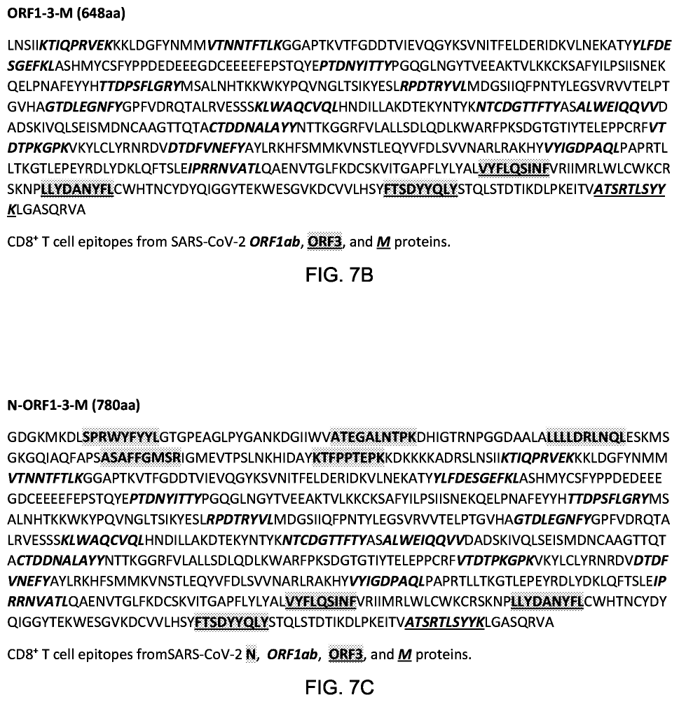

An epitope string is an engineered polypeptide comprising linked epitopes of any one or more proteins. For example, an epitope may be identified within a full protein sequence, and amino acids between epitopes may be deleted partially or fully (see, e.g., ORF1-3-M and N-ORF1-3-M described below, providing sequences of epitope strings derived from the SARS-CoV-2 N, ORF1ab, ORF3, and M proteins), or replaced with spacer polypeptides. The epitope string need only function as an immunogen, and therefore structure and function of the protein from which the epitope is derived need not be conserved, permitting significant flexibility in inter-epitope deletions and substitutions in the spacer sequence between the defined epitopes. For example, and epitope string may be produced by deleting one or more amino acids from a native amino acid sequence, and/or combining epitopes, and optionally flanking sequences in frame from multiple native proteins, as in the epitope strings of FIGS. 7B and 7C, comprising CD8⁺ epitopes including flanking (e.g., from six to ten) native N-terminal and C-terminal amino acids as spacers.

Ferretti A P, et al. provides an exemplary list of CD8⁺ T cell epitope sequences identified in SARS-CoV-2 sequences. The immunogen may therefore comprise one or more of the following CD8⁺ T cell epitope sequences identified in Ferretti A P, et al. (see, FIG. 7A): KLWAQCVQL (SEQ ID NO: 16), YLQPRTFLL (SEQ ID NO: 17), LLYDANYFL (SEQ ID NO: 18), ALWEIQQVV (SEQ ID NO: 19), LLLDRLNQL (SEQ ID NO: 20), YLFDESGEFKL (SEQ ID NO: 21), FTSDYYQLY (SEQ ID NO: 22), TTDPSFLGRY (SEQ ID NO: 23), PTDNYITTY (SEQ ID NO: 24), ATSRTLSYY (SEQ ID NO: 25), CTDDNALAYY (SEQ ID NO: 26), NTCDGTTFTY (SEQ ID NO: 27), DTDFVNEFY (SEQ ID NO: 28), GTDLEGNFY (SEQ ID NO: 29), KTFPPTEPK (SEQ ID NO: 30), KCYGVSPTK (SEQ ID NO: 31), VTNNTFTLK (SEQ ID NO: 32), KTIQPRVEK (SEQ ID NO: 33), KTFPPTEPK (SEQ ID NO: 34), VTDTPKGPK (SEQ ID NO: 35), ATEGALNTPK (SEQ ID NO: 36), ASAFFGMSR (SEQ ID NO: 37), ATSRTLSYYK (SEQ ID NO: 38), QYIKWPWYI (SEQ ID NO: 39), VYFLQSINF (SEQ ID NO: 40), VYIGDPAQL (SEQ ID NO: 41), SPRWYFYYL (SEQ ID NO: 42), RPDTRYVL (SEQ ID NO: 43), or IPRRNVATL (SEQ ID NO: 44), separated by spacer amino acid sequence(s). The epitope string may comprise one or more repeats of a single epitope amino acid sequence, and/or may comprise multiple different amino acid sequences.

An immune response is a response of a cell of the immune system, such as a B-cell, T-cell, macrophage or polymorphonucleocyte, to a stimulus, such as an antigen. An immune response may include any cell of the body involved in a host defense response for example, an epithelial cell that secretes an interferon or a cytokine. An immune response includes, but is not limited to, an innate immune response or inflammation.

To immunize a subject refers to rendering a subject protected from an infectious disease, such as by vaccination.

An immunogen refers to a compound, composition, or substance which is capable, under appropriate conditions, of stimulating an immune response, such as the production of antibodies, such as neutralizing antibodies, or a T-cell response in an animal, including compositions that are injected or absorbed into an animal. As used herein, an “immunogenic composition” is a composition comprising an immunogen (such as a coronavirus S1 polypeptide or an epitope string comprising two or more epitopes), which may have the same or different amino acid sequences.

An immunoglobulin Fc domain is a polypeptide including the constant region of an antibody excluding the first constant region immunoglobulin domain. Fc domain generally refers to the last two constant region immunoglobulin domains of IgA, IgD, and IgG, and the last three constant region immunoglobulin domains of IgE and IgM. An Fc domain may also include part or all of the flexible hinge N-terminal to these domains. For IgA and IgM, an Fc domain may or may not include the tailpiece, and may or may not be bound by the J chain. For IgG, the Fc domain includes immunoglobulin domains Cgamma2 and Cgamma3 (Cγ2 and Cγ3) and the lower part of the hinge between Cgamma1 (Cγ1) and Cγ2. Although the boundaries of the Fc domain may vary, the human IgG heavy chain Fc domain is usually defined to include residues C226 or P230 to its carboxyl-terminus, wherein the numbering is according to the EU index as in Kabat. For IgA, the Fc domain includes immunoglobulin domains Calpha2 and Calpha3 (Cα2 and Cα3) and the lower part of the hinge between Calpha1 (Cα1) and Cα2.

An “isolated” or “purified” biological component (such as a nucleic acid, peptide, protein, protein complex, or particle) refers to a component that has been substantially separated, produced apart from, or purified away from other components in a preparation or other biological components in the cell of the organism in which the component occurs, that is, other chromosomal and extrachromosomal DNA and RNA, and proteins. Nucleic acids, peptides and proteins that have been “isolated” or “purified”, thus, include nucleic acids and proteins purified by standard purification methods. The term also embraces nucleic acids, peptides and proteins prepared by recombinant expression in a host cell, as well as chemically synthesized nucleic acids or proteins. The term “isolated” or “purified” does not require absolute purity; rather, it is intended as a relative term. Thus, for example, an isolated biological component is one in which the biological component is more enriched than the biological component is in its natural environment within a cell, or other production vessel. A preparation may be purified such that the biological component represents at least 50%, such as at least 70%, at least 90%, at least 95%, or greater, of the total biological component content of the preparation.

A linker refers to a molecule or group of atoms positioned between two moieties (portions of a molecule). Typically, linkers are bifunctional, e.g., the linker includes a functional group at each end, wherein the functional groups are used to couple the linker to the two moieties. The two functional groups may be the same, e.g., a homobifunctional linker, or different, e.g., a heterobifunctional linker. A peptide linker may be used to link the C-terminus of a first polypeptide to the N-terminus of a second polypeptide in a fusion protein or peptide. Non-limiting examples of peptide linkers include glycine-serine peptide linkers, which are typically not more than 10 amino acids in length. Typically, such linkage is accomplished using molecular biology techniques to genetically manipulate DNA encoding the first polypeptide linked to the second polypeptide by the peptide linker in an open reading frame.

A nucleic acid molecule (a nucleic acid) refers to a polymeric form of nucleotides, which may include both sense and anti-sense strands of RNA, cDNA, genomic DNA, and synthetic forms and mixed polymers of the above. A nucleotide refers to a ribonucleotide, deoxynucleotide or a modified form of either type of nucleotide. The term “nucleic acid molecule” as used herein is synonymous with “nucleic acid” and “polynucleotide.” The term includes single- and double-stranded forms of DNA. A polynucleotide may include either or both naturally occurring and modified nucleotides linked together by naturally occurring and/or non-naturally occurring nucleotide linkages.

A first nucleic acid is said to be operably linked to a second nucleic acid when the first nucleic acid is placed in a functional relationship with the second nucleic acid. Generally, operably linked DNA sequences are contiguous (e.g., in cis) and, where the sequences act to join two protein coding regions, in the same reading frame. Operably linked nucleic acids include a first nucleic acid contiguous with the 5′ or 3′ end of a second nucleic acid. In other examples, a second nucleic acid is operably linked to a first nucleic acid when it is embedded within the first nucleic acid, for example, where the nucleic acid construct includes (in order) a portion of the first nucleic acid, the second nucleic acid, and the remainder of the first nucleic acid.

A polypeptide is a polymer in which the monomers are amino acid residues which are joined together through amide bonds. When the amino acids are alpha-amino acids, either the L-optical isomer or the D-optical isomer can be used. The terms “polypeptide” or “protein” as used herein are intended to encompass any amino acid sequence and include proteins and modified sequences such as glycoproteins. The term “polypeptide” is specifically intended to cover naturally occurring proteins, as well as those which are recombinantly or synthetically produced. The term “residue” or “amino acid residue” includes reference to an amino acid that is incorporated into a protein, polypeptide, or peptide.

Conservative amino acid substitutions are those substitutions that, when made, least interfere with the properties of the original protein, that is, the structure and especially the function of the protein is conserved and not significantly changed by such substitutions, and may be identified by use of matrices, such as the BLOSUM series of matrices, and other matrices.

Conservative substitutions generally maintain (a) the structure of the polypeptide backbone in the area of the substitution, for example, as a sheet or helical conformation, (b) the charge or hydrophobicity of the molecule at the target site, or (c) the bulk of the side chain.

The substitutions which in general are expected to produce the greatest changes in protein properties will be non-conservative, for instance changes in which (a) a hydrophilic residue, for example, seryl or threonyl, is substituted for (or by) a hydrophobic residue, for example, leucyl, isoleucyl, phenylalanyl, valyl, or alanyl; (b) a cysteine or proline is substituted for (or by) any other residue; (c) a residue having an electropositive side chain, for example, lysyl, arginyl, or histadyl, is substituted for (or by) an electronegative residue, for example, glutamyl or aspartyl; or (d) a residue having a bulky side chain, for example, phenylalanine, is substituted for (or by) one not having a side chain, for example, glycine.

A promoter is an array of nucleic acid control sequences which direct transcription of a nucleic acid. A promoter includes necessary nucleic acid sequences near the start site of transcription. A promoter also optionally includes distal enhancer or repressor elements. A “constitutive promoter” is a promoter that is continuously active and is not subject to regulation by external signals or molecules. In contrast, the activity of an “inducible promoter” is regulated by an external signal or molecule (for example, a transcription factor).

A recombinant nucleic acid refers to a nucleic acid molecule (or protein or virus) that is not naturally occurring or has a sequence that is made by an artificial combination of two otherwise separated segments of sequence. This artificial combination is accomplished by chemical synthesis or, more commonly, by the artificial manipulation of isolated segments of nucleic acids, e.g., by genetic engineering techniques such as those described in Sambrook et al., (ed.), Molecular Cloning: A Laboratory Manual, 2nd ed., vol. 1-3, Cold Spring Harbor Laboratory Press, Cold Spring Harbor, N.Y., 1989. The term recombinant includes nucleic acids and proteins that have been altered solely by addition, substitution, or deletion of a portion of a natural nucleic acid molecule or protein.

“Sequence identity” refers to the similarity between nucleic acid or amino acid sequences is expressed in terms of the similarity between the sequences, otherwise referred to as sequence identity. Sequence identity may be measured in terms of percentage identity (or similarity or homology); the higher the percentage, the more similar the two sequences are. Homologs, orthologs, or variants of a polypeptide will possess a relatively high degree of sequence identity when aligned using standard methods. Methods of alignment of sequences for comparison are well-known in the art. Various programs and alignment algorithms are described in the art.: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al., Computer Appls. In the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

Once aligned, the number of matches may be determined by counting the number of positions where an identical nucleotide or amino acid residue is present in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a peptide sequence that has 1166 matches when aligned with a test sequence having 1554 amino acids is 75.0 percent identical to the test sequence (1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer.

Homologs and variants of a polypeptide are typically characterized by possession of at least about 75%, for example, at least about 80%, 85%, 90%, 91%, 92%, 93%, 94%, 95%, 96%, 97%, 98%, or 99% sequence identity counted over the full length alignment with the amino acid sequence of interest. Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

For sequence comparison of nucleic acid sequences, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters are used. Methods of alignment of sequences for comparison are well known in the art. Optimal alignment of sequences for comparison can be conducted, e.g., by the local homology algorithm of Smith & Waterman, Adv. Appl. Math. 2:482, 1981, by the homology alignment algorithm of Needleman & Wunsch, J. Mol. Biol. 48:443, 1970, by the search for similarity method of Pearson & Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Sambrook et al. (Molecular Cloning: A Laboratory Manual, 4th ed, Cold Spring Harbor, N.Y., 2012) and Ausubel et al. (In Current Protocols in Molecular Biology, John Wiley & Sons, New York, through supplement 104, 2013)). One example of a useful algorithm is PILEUP. PILEUP uses a simplification of the progressive alignment method of Feng & Doolittle, J. Mol. Evol. 35:351-360, 1987. The method used is similar to the method described by Higgins & Sharp, CABIOS 5:151-153, 1989. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., Nuc. Acids Res. 12:387-395, 1984).

Another example of algorithms that are suitable for determining percent sequence identity and sequence similarity are the BLAST and the BLAST 2.0 algorithm, which are described in Altschul et al., J. Mol. Biol. 215:403-410, 1990 and Altschul et al., Nucleic Acids Res. 25:3389-3402, 1977. Software for performing BLAST analyses is publicly available through the National Center for Biotechnology Information (ncbi.nlm.nih.gov). The BLASTN program (for nucleotide sequences) uses as defaults a word length (W) of 11, alignments (B) of 50, expectation (E) of 10, M=5, N=−4, and a comparison of both strands. The BLASTP program (for amino acid sequences) uses as defaults a word length (W) of 3, and expectation (E) of 10, and the BLOSUM62 scoring matrix (see Henikoff & Henikoff, Proc. Natl. Acad. Sci. USA 89:10915, 1989). An oligonucleotide is a linear polynucleotide sequence of up to about 100 nucleotide bases in length.

As used herein, reference to “at least 80% identity” (or similar language) refers to “at least 80%, at least 85%, at least 90%, at least 91%, at least 92%, at least 93%, at least 94%, at least 95%, at least 96%, at least 97%, at least 98%, at least 99%, or even 100% identity” to a specified reference sequence. As used herein, reference to “at least 90% identity” (or similar language) refers to “at least 90%, at least 91%, at least 92%, at least 93%, at least 94%, at least 95%, at least 96%, at least 97%, at least 98%, at least 99%, or even 100% identity” to a specified reference sequence.

Complementary refers to the ability of polynucleotides (nucleic acids) to hybridize to one another, forming inter-strand base pairs. Base pairs are formed by hydrogen bonding between nucleotide units in polynucleotide strands that are typically in antiparallel orientation. Complementary polynucleotide strands can base pair (hybridize) in the Watson-Crick manner (e.g., A to T, A to U, C to G), or in any other manner that allows for the formation of duplexes. In RNA as opposed to DNA, uracil rather than thymine is the base that is complementary to adenosine. Two sequences comprising complementary sequences can hybridize if they form duplexes under specified conditions, such as in water, saline (e.g., normal saline, or 0.9% w/v saline) or phosphate-buffered saline), or under other stringency conditions, such as, for example and without limitation, 0.1×SSC (saline sodium citrate) to 10×SSC, where 1×SSC is 0.15M NaCl and 0.015M sodium citrate in water. Hybridization of complementary sequences is dictated, e.g., by the nucleobase content of the strands, the presence of mismatches, the length of complementary sequences, salt concentration, temperature, with the melting temperature (Tm) lowering with shorter complementary sequences, increased mismatches, and increased stringency. Perfectly matched sequences are said to be “fully complementary”, though one sequence (e.g., a target sequence in an mRNA) may be longer than the other.

A “transformed” cell is a cell into which has been introduced a nucleic acid molecule (such as a heterologous nucleic acid) by any useful molecular biology technique. The term encompasses all techniques by which a nucleic acid molecule might be introduced into such a cell, including, without limitation, transfection with viral vectors, transformation with plasmid vectors, and introduction of naked DNA by electroporation, lipofection, or particle gun acceleration.

A vaccine refers to a preparation of immunogenic material capable of stimulating an immune response, administered for the prevention, inhibition, amelioration, or treatment of infectious, such as coronavirus, e.g. SARS-CoV-2, infections, or other types of disease. The immunogenic material may include attenuated or inactivated (killed) microorganisms (such as bacteria or viruses), or antigenic proteins, peptides or DNA derived from them. An attenuated virus is a virulent organism that has been modified to produce a less virulent form, but nevertheless retains the ability to elicit antibodies and cell-mediated immunity against the virulent form. An inactivated (killed) virus is a previously virulent organism that has been inactivated with chemicals, heat, or other treatment, but elicits antibodies against the organism. Vaccines may elicit both prophylactic (preventative or protective) and therapeutic responses. Methods of administration vary according to the vaccine, but may include inoculation, ingestion, inhalation or other forms of administration. Vaccines may be administered with an adjuvant to boost the immune response.

A vector is a nucleic acid molecule allowing insertion of foreign nucleic acid without disrupting the ability of the vector to replicate and/or integrate in a host cell. A vector can include nucleic acid sequences that permit it to replicate in a host cell, such as an origin of replication. An insertional vector is capable of inserting itself into a host nucleic acid. A vector can also include one or more selectable marker genes and other genetic elements. An expression vector is a vector that contains the necessary regulatory sequences to allow transcription and translation of inserted gene or genes.

By “expression” or “gene expression,” it is meant the overall flow of information from a gene or functional/structural RNA, and a polyadenylation sequence), to produce a gene product (typically a protein, optionally post-translationally modified or a functional/structural RNA). A “gene” refers to a functional genetic unit for producing a gene product, such as RNA or a protein in a cell, or other expression system encoded on a nucleic acid and comprising: a transcriptional control sequence, such as a promoter and other cis-acting elements, such as transcriptional response elements (TREs) and/or enhancers; an expressed sequence that may encode a protein (referred to as an open-reading frame or ORF), and a polyadenylation sequence. By “expression of genes under transcriptional control of,” or alternately “subject to control by,” a designated sequence such as TRE or transcription control element, it is meant gene expression from a gene containing the designated sequence operably linked (functionally attached, typically in cis) to the gene. A “gene for expression of” a stated gene product is a gene capable of expressing that stated gene product when placed in a suitable environment—that is, for example, when transformed, transfected, transduced, etc. into a cell, and subjected to suitable conditions for expression. In the case of a constitutive promoter “suitable conditions” means that the gene typically need only be introduced into a host cell. In the case of an inducible promoter, “suitable conditions” means when factors that regulate transcription, such as DNA-binding proteins, are present or absent—for example an amount of the respective inducer is available to the expression system (e.g., cell), or factors causing suppression of a gene are unavailable or displaced—effective to cause expression of the gene.

A coronavirus polypeptide is a polypeptide encoded by a coronavirus or a portion of a polypeptide encoded by a coronavirus, as in epitopes and immunogenic fragments thereof, and polypeptides with significant sequence identity (e.g., at least 85%, 90%, 95%, 98%, or 99%) with a polypeptide encoded by a natural coronavirus, and retaining or improving upon native function as an immunogen or epitope.

Unless otherwise explained, all technical and scientific terms used herein have the same meaning as commonly understood by one of ordinary skill in the art to which this disclosure belongs. It is to be understood that all base sizes or amino acid sizes, and all molecular weight or molecular mass values, given for nucleic acids or polypeptides are approximate, and are provided for description. Unless otherwise indicated, polymer molecular weight is expressed as number-average molecular weight (Mn). Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

Immunogens are disclosed herein. These immunogens may be used to induce a neutralizing immune response, and were shown to protect against coronavirus or SARS-CoV-2 challenge in an animal model of a coronavirus or SARS-CoV-2 infection. As shown in the Examples below, the immunogens include a native protein or a portion of a native protein, such as a coronavirus or SARS-CoV-2 S1 and/or nucleoprotein, or a fragment of either, and may be a fusion protein comprising two or more native proteins or fragments thereof, such as a coronavirus or SARS-CoV-2 S1 protein or a fragment thereof linked in-frame with a coronavirus or SARS-CoV-2 nucleoprotein, M protein, ORF1ab protein, and/or ORF3 protein, or a fusion protein or fragment thereof. An immunogen may be delivered or administered to a patient as a protein, or as a nucleic acid for expressing the protein, such as an mRNA encoding the protein, or as an engineered nucleic acid, such as naked DNA or a viral vector comprising a gene for expressing an immunogen.

The immunogen may comprise an epitope string comprising epitopes of a natural protein, such as epitopes of a coronavirus or SARS-CoV-2 N, M, ORF1ab, and/or ORF3 fusion protein. A signal peptide may be included in the immunogen, such as a premembrane (prM) signal peptide, an IgG signal peptide, or a human secretory signal peptide hidden Markov model. A multimerization domain may be included, such as an immunoglobulin Fc domain (see, e.g., Yang. C. et al., Engineering of Fc Fragments with Optimized Physicochemical Properties Implying Improvement of Clinical Potentials for Fc-Based Therapeutics. Front. Immunol. 8:1860, 8 Jan. 2018, https://doi.org/10.3389/fimmu.2017.01860), a T4 fibritin foldon trimerization domain (see, e.g., Papanikolopoulou, K., et al., “Formation of Highly Stable Chimeric Trimers by Fusion of an Adenovirus Fiber Shaft Fragment with the Foldon Domain of Bacteriophage T4 Fibritin” J. Biol. Chem. 2004 279: 8991. doi:10.1074/jbc.M311791200), or a human collagen XV trimerization domain (See, e.g., Ángel M. Cuesta et al., (2012) Improved stability of multivalent antibodies containing the human collagen XV trimerization domain, mAbs, 4:2, 226-232). Any combination of these domains may be utilized.

A multimerization domain may be placed at the C-terminus of the immunogen in the fusion protein. Suitable multimerization domains include, but are not limited to, the following exemplary sequences:

1. Immunoglobulin Dimerization Domain
(SEQ ID NO: 63)
DKTHTCPSRPCPAPELLGGPSVFLFPPKPKDTLMISRTPEVTCVVVDVSH
EDPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQ
QGNVFSCSVLHEALHSHYTQKSLSLSPGK;
2. T4 Fibritin Foldon Trimerization Domain
(SEQ ID NO: 64)
GYIPEAPRDGQAYVRKDGEWVLLSTFL;
and
3. Human Collagen XV Trimerization Domain
(SEQ ID NO: 65)
VTAFSNMDDMLQKAHLVIEGTFIYLRDSTEFFIRVRDGWKKLQLGELIPI
PADSPPPPALSSNP.

A multimerization domain may include an amino acid sequence at least 95% identical to any one of the sequences provided in the preceding paragraph, such as an amino acid sequence about 95%, about 96%, about 97%, about 98%, about 99%, or 100% identical to any one of the sequences provided in the preceding paragraph, provided the multimerization domain functions, such that dimers or trimers are produced (as appropriate to the native domain). A multimerization domain can include at most 1, 2, 3, or 4 conservative amino acid substitutions in one of any one of the sequences provided in the preceding paragraph, provided the multimerization domain functions, such that dimers or trimers are produced (as appropriate to the native domain). In some embodiments, the multimerization domain consists of the amino acid sequence of any one of the sequences provided in the preceding paragraph.

A Toll-like receptor (TLR) agonist adjuvant, e.g., a Toll-like receptor 4 (TLR4) or Toll-like receptor 3 (TLR3) agonist (alt. ligand) domain may be included in the fusion protein. Sequences of such TLR agonists are known. In one example, TLR3 ligands activate keratinocytes, innate immune cells, and professional APCs and induce cross-presentation of antigen to prime CD8⁺ T cells (Datta et al., “A subset of toll-like receptor ligands induces cross-presentation by bone marrow-derived dendritic cells”, 2003, J. Immunol., 170: 4102-4110; Schulz et al., “Toll-like receptor 3 promotes cross-priming to virus-infected cells”, 2005, Nature, 433:887-892; Kalali et al., “Double-stranded RNA induces an antiviral defense status in epidermal keratinocytes through TLR3-, PKR-, and MDA5/RIG-I-mediated differential signaling”, 2008, J. Immunol., 181: 2694-2704). To improve the immunogenicity of the vaccines, we constructed S1 subunits with integrated Toll-like receptor (TLR) agonist sequences, specifically RS09 and Flagellin C. RS09 is a synthetic form of an LPS mimic 7-mer peptide, which binds to TLR4 and induces nuclear localization of the transcription factor NF-κB, resulting in transcription of inflammatory cytokines and secretion of chemokines (Shanmugam et al., “Synthetic Toll like receptor-4 (TLR-4) agonist peptides as a novel class of adjuvants”, 2012, PloS one; 7(2): e30839). Flagellin is a highly conserved natural bacterial ligand that binds to TLR5 to activate TLR5 expressing dendritic cells, neutrophils, lung epithelial cells and pneumonocytes, augmenting immunogenicity in several vaccine models (Song et al., “An avian influenza A (H7N9) virus vaccine candidate based on the fusion protein of hemagglutinin globular head and Salmonella typhimurium flagellin”, 2015, BMC biotechnology 2015; 15: 79; Lopez-Boado et al., “Bordetella bronchiseptica flagellin is a proinflammatory determinant for airway epithelial cells”, 2005, Infection and immunity; 73(11): 7525-34; Skountzou et al., “Salmonella flagellins are potent adjuvants for intranasally administered whole inactivated influenza vaccine”, 2010, Vaccine; 28(24): 4103-12).

In addition, or alternatively to the inclusion of the TLR agonist sequence in the fusion protein, a Poly(I:C) adjuvant (polyinosinic-polycytidylic acid (poly(I:C))) or Poly-ICLC (poly(I:C) stabilized with poly lysine and carboxymethylcellulose) also may be included as an adjuvant in a pharmaceutical composition as described herein (See, e.g., Tewari K, Flynn B J, Boscardin S B, et al., “Poly(I:C) is an effective adjuvant for antibody and multi-functional CD4⁺ T cell responses to Plasmodium falciparum circumsporozoite protein (CSP) and aDEC-CSP in non-human primates.” Vaccine. 2010; 28(45):7256-7266. doi:10.1016/j.vaccine.2010.08.098).

Additional amino acid sequences, including linkers, spacers, carriers (see, e.g., US 2020/0031874), signal peptides, self-cleaving sequences, and affinity tags may be included in the fusion protein, so long as they do not interfere to any significance with the operation of the immunogen in eliciting an appropriate immune response to a coronavirus, e.g., SARS-CoV-2. Portions of the coronavirus spike protein, e.g., the S1 protein, the nucleoprotein, and/or other coronavirus proteins, may be utilized so long as immunogenicity is substantially retained. The polypeptide may include two or more iterations of a spike protein sequence, a nucleoprotein sequence, other coronavirus sequences, or immunogenic portions thereof, arranged in frame in the fusion protein. The two or more iterations of the spike protein, nucleoprotein protein, or other coronavirus protein, or immunogenic portions thereof, may have the same sequence, or different sequences, e.g., accounting for genetic variation of the protein(s). Likewise, where the immunogen comprises an instance of the spike protein, nucleoprotein, or other coronavirus protein(s), or immunogenic portions thereof, in a single polypeptide chain, polypeptide chains with different sequences accounting for genetic variation of the proteins may be prepared and co-multimerized to produce a multi-valent immunogen.

The immunogens may be delivered as a protein, as an mRNA, or as a gene, for example in the form of naked DNA, or included in a recombinant virus genome, for example as Adenoviral vectors, e.g., Ad5 vectors, described herein. A viral genome, in reference to recombinant adenovirus constructs, refers to a packageable nucleic acid sequence or a packaged nucleic acid sequence within, and deliverable by, a virus particle, such as a replication-defective infectious unit.

Nucleic acids and vectors encoding these fusion proteins may be provided. In some non-limiting examples, disclosed is a recombinant vector, such as an adenoviral vector, the expresses the disclosed immunogens. Polynucleotides encoding a disclosed immunogen are also provided. These polynucleotides include DNA, cDNA, and RNA sequences which encode the antigen. One of skill in the art can readily use the genetic code to construct a variety of functionally equivalent nucleic acids, such as nucleic acids which differ in sequence but which encode the same protein sequence, or encode a conjugate or fusion protein including the nucleic acid sequence. In some embodiments, the polynucleotide is codon-optimized for expression in human cells. In specific non-limiting examples, nucleic acids encoding a coronavirus, e.g., SARS-CoV-2, immunogen as described herein can be codon optimized.

Exemplary nucleic acids may be prepared by cloning techniques, e.g., as are broadly-known and implemented either commercially, or in the art. Multiple textbooks and reference manuals describe and provide examples of useful and appropriate cloning and sequencing techniques, and instructions sufficient to direct persons of skill through such techniques are known. Commercial and public product information from manufacturers of biological reagents and experimental equipment also provide useful information. Such manufacturers include the SIGMA Chemical Company (Saint Louis, Mo.), R&D Systems (Minneapolis, Minn.), Pharmacia Amersham (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersburg, Md.), Fluka Chemica-Biochemika Analytika (Fluka Chemie AG, Buchs, Switzerland), Invitrogen (Carlsbad, Calif.), Addgene, and Applied Biosystems (Foster City, Calif.), as well as many other commercial sources.

The disclosed immunogens and viral vectors can be delivered to a subject to produce an immune response to a coronavirus, such as a SARS-CoV-2 virus, such as a protective or neutralizing immune response. In some embodiments, delivery can be transcutaneously by microneedle arrays (MNAs), such as carboxymethyl cellulose- (CMC-) containing MNAs.

Nucleic acids can also be prepared by amplification methods. Amplification methods include polymerase chain reaction (PCR), the ligase chain reaction (LCR), the transcription-based amplification system (TAS), the self-sustained sequence replication system (3SR). A wide variety of cloning methods, host cells, and in vitro amplification methodologies are well known to persons of skill.

The polynucleotides encoding a disclosed immunogen can include a recombinant DNA which is incorporated into a vector into an autonomously replicating plasmid or virus or into the genomic DNA of a prokaryote or eukaryote, or which exists as a separate molecule (such as a cDNA) independent of other sequences. The nucleotides can be ribonucleotides, deoxyribonucleotides, or modified forms of either nucleotide. The term includes single- and double-stranded forms of DNA.

Polynucleotide sequences encoding a disclosed immunogen can be operatively linked to expression control sequences. An expression control sequence operatively linked to a coding sequence is placed in the sequence such that expression of the coding sequence is achieved under conditions compatible with the expression control sequences. The expression control sequences include, but are not limited to, appropriate promoters, enhancers, transcription terminators, a start codon (e.g., ATG) in front of a protein-encoding gene, splicing signal for introns, maintenance of the correct reading frame of that gene to permit proper translation of mRNA, and stop codons.

DNA sequences encoding the disclosed immunogen can be expressed in vitro by DNA transfer into a suitable host cell. The cell may be prokaryotic or eukaryotic. The term also includes any progeny of the subject host cell. It is understood that all progeny may not be identical to the parental cell since there may be mutations that occur during replication. Methods of stable transfer, meaning that the foreign DNA is continuously maintained in the host, are known in the art.

Hosts can include microbial, yeast, insect and mammalian organisms. Methods of expressing DNA sequences having eukaryotic or viral sequences in prokaryotes are well known in the art. Non-limiting examples of suitable host cells include bacteria, archea, insect, fungi (for example, yeast), plant, and animal cells (for example, mammalian cells, such as human). Exemplary cells of use include Escherichia coli, Bacillus subtilis, Saccharomyces cerevisiae, Salmonella typhimurium, SF9 cells, C129 cells, 293 cells, Neurospora, and immortalized mammalian myeloid and lymphoid cell lines. Techniques for the propagation of mammalian cells in culture are well-known (see, e.g., Helgason and Miller (Eds.), 2012, Basic Cell Culture Protocols (Methods in Molecular Biology), 4th Ed., Humana Press). Examples of commonly used mammalian host cell lines are VERO and HeLa cells, CHO cells, and W138, BHK, and COS cell lines, although cell lines may be used, such as cells designed to provide higher expression, desirable glycosylation patterns, or other features. In some embodiments, the host cells include HEK293 cells or derivatives thereof, such as GnTI^{-/-} cells (ATCC® No. CRL-3022), or HEK-293F cells.

Transformation of a host cell with recombinant DNA can be carried out by conventional techniques as are well known to those skilled in the art. Where the host is prokaryotic, such as, but not limited to, E. coli, competent cells which are capable of DNA uptake can be prepared from cells harvested after exponential growth phase and subsequently treated by the CaCl₂ method using procedures well known in the art. Alternatively, MgCl₂ or RbCl can be used. Transformation can also be performed after forming a protoplast of the host cell if desired, or by electroporation.

When the host is a eukaryote, such methods of transfection of DNA as calcium phosphate co-precipitates, conventional mechanical procedures such as microinjection, electroporation, insertion of a plasmid encased in liposomes, or viral vectors can be used. Eukaryotic cells can also be co-transformed with polynucleotide sequences encoding a disclosed antigen, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein. One of skill in the art can readily use an expression systems such as plasmids and vectors for use in producing proteins in cells including higher eukaryotic cells such as the COS, CHO, HeLa, and myeloma cell lines.

Modifications may be made to a nucleic acid encoding a disclosed immunogen without diminishing its biological activity. Some modifications may be made to facilitate the cloning, expression, or incorporation of the targeting molecule into a fusion protein. Such modifications include, for example, termination codons, a methionine added at the amino terminus to provide an initiation, site, additional amino acids placed on either terminus to create conveniently located restriction sites, or additional amino acids (such as poly His) to aid in purification steps.

A nucleic acid molecule encoding a disclosed immunogen may be included in a viral vector, for example, for expression of the immunogen in a host cell, or for immunization of a subject as disclosed herein. In some embodiments, the viral vectors are administered to a subject as part of a prime-boost vaccination protocol. The viral vectors may be included in a vaccine, such as a primer vaccine or a booster vaccine for use in a prime-boost vaccination.

Adenoviral vectors are able to transduce both replicating and quiescent cell populations, making them a valuable tool in delivering transgenes in vivo and within mature tissues. A replication-defective adenovirus infectious unit, plaque-forming unit, or particle comprises a nucleocapsid including fiber, penton, and hexon proteins where the fibers facilitate attachment to the coxsackie-Ad receptor (CAR), and is a virion that includes a adenovirus viral capsid and a double-stranded DNA genome comprising at least inverted terminal repeats, a packaging signal ((P), and a gene encoding a protein or functional RNA to be expressed. Multiple generations of Ad vectors have been developed, capable of incorporating transgenes of varying sizes. Most of the current Ad vectors are derived from human adenovirus, type-5, which is rare in humans. The Ad vectors are replication-defective and remain extrachromosomal. This means they cannot, by themselves, complete the viral life-cycle, and therefore act only as transducing particles for extrachromosomal, transient expression of the gene or genes carried therein. First generation Ad vectors are those stripped of regulatory genes Ela and E1 b, which are the first transcriptional regulatory factors produced during the viral life cycle. The depletion of this gene resulted in replication-deficient Ad vector with an initial transgene cloning capacity of about 5.2 kb. Second generation Ad vectors are vectors with deletion of other non-structural genes (E2/E3/E4) in addition to deletion of the original E1 gene absent in first generation vectors. Third generation Ad vectors, are also may be referred to as gutless Ad vectors, high-capacity Ad vectors, or helper-dependent Ad vectors. Gutless Ad vectors are stripped of all viral coding sequences, resulting in a vector with only 5′ and 3′ ITRs in addition to a packaging signal ((P), thus resulting in a larger capacity for transgenic cloning sequences (e.g., up to 36 kb). The structure of the gutless Ad vector minimizes cytotoxicity, thus enabling prolonged expression of therapeutic genes, rendering it the most promising Ad vector for therapeutic use. (See, e.g., Lee, C. S. et al., “Adenovirus-Mediated Gene Delivery: Potential Applications for Gene and Cell-Based Therapies in the New Era of Personalized Medicine.” Genes & diseases vol. 4, 2 (2017): 43-63). In the context of adenovirus, adenovirus vectors and infectious units, e.g., replication-defective adenovirus vectors and infectious units, a genome is the nucleic acid that is packaged in the infectious unit, including, at least ITRs, Ψ, and a gene for expressing a protein or RNA, such as a gene for expressing a polypeptide or protein comprising an immunogenic amino acid sequence of a pathogen, such as an immunogenic amino acid sequence of SARS-CoV-2, for example and without limitation the SARS-CoV-2 spike protein.

Adenovirus from various origins, subtypes, or mixture of subtypes can be used as the source of the viral genome for the adenoviral vector. Non-human adenovirus (e.g., simian, chimpanzee, gorilla, avian, canine, ovine, or bovine adenoviruses) may be used to generate the adenoviral vector. For example, a simian adenovirus can be used as the source of the viral genome of the adenoviral vector. A simian adenovirus may be of serotype 1, 3, 7, 11, 16, 18, 19, 20, 27, 33, 38, 39, 48, 49, 50, or any other simian adenoviral serotype. A simian adenovirus can be referred to by using any suitable abbreviation known in the art, such as, for example, SV, SAdV, SAV or sAV. In some examples, a simian adenoviral vector is a simian adenoviral vector of serotype 3, 7, 11, 16, 18, 19, 20, 27, 33, 38, or 39. A chimpanzee serotype C Ad3 vector may be used (see, e.g., Peruzzi et al., Vaccine, 27:1293-1300, 2009) or an Ad5 vector is used (see the Examples Section). Human adenovirus can be used as the source of the viral genome for the adenoviral vector. Human adenovirus can be of various subgroups or serotypes. For instance, an adenovirus can be of subgroup A (e.g., serotypes 12, 18, and 31), subgroup B (e.g., serotypes 3, 7, 11, 14, 16, 21, 34, 35, and 50), subgroup C (e.g., serotypes 1, 2, 5, and 6), subgroup D (e.g., serotypes 8, 9, 10, 13, 15, 17, 19, 20, 22, 23, 24, 25, 26, 27, 28, 29, 30, 32, 33, 36-39, and 42-48), subgroup E (e.g., serotype 4), subgroup F (e.g., serotypes 40 and 41), an unclassified serogroup (e.g., serotypes 49 and 51), or any other adenoviral serotype. The person of ordinary skill in the art is familiar with replication competent and deficient adenoviral vectors (including singly and multiply replication deficient adenoviral vectors). Examples of replication-deficient adenoviral vectors, including multiply replication-deficient adenoviral vectors, are disclosed in U.S. Pat. Nos. 5,837,511; 5,851,806; 5,994,106; 6,127,175; 6,482,616; and 7,195,896, and International Patent Application Nos. WO 94/28152, WO 95/02697, WO 95/16772, WO 95/34671, WO 96/22378, WO 97/12986, WO 97/21826, and WO 03/022311.

The compositions, methods, and dosage forms disclosed herein may include one or more adjuvants or molecules with immune stimulant or adjuvant effect. In other examples, an adjuvant is not included in the composition, but is separately administered to a subject (for example, in combination with a composition disclosed herein) before, after, or substantially simultaneously with administration of one or more of the immunogen-containing compositions disclosed herein. Adjuvants are agents that increase or enhance an immune response in a subject administered an antigen, compared to administration of the antigen in the absence of an adjuvant. One example of an adjuvant is an aluminum salt, such as aluminum hydroxide, aluminum phosphate, aluminum potassium sulfate, or aluminum hydroxyphosphate. Other adjuvants include biological adjuvants, such as cytokines (for example, IL-2, IL-6, IL-12, RANTES, GM-CSF, TNF-α, or IFN-γ), growth factors (for example, GM-CSF or G-CSF), one or more molecules such as OX-40L or 4-1 BBL, immunostimulatory oligonucleotides (for example, CpG oligonucleotides, for example, see U.S. Pat. Nos. 6,194,388; 6,207,646; 6,214,806; 6,218,371; 6,239,116; 6,339,068; 6,406,705; and 6,429,199), Toll-like receptor agonists (for example, TLR2, TLR4, TLR7/8, or TLR9 agonists), and bacterial lipopolysaccharides or their derivatives (such as 3D-MPL). Additional adjuvants include oil and water emulsions, squalene, or other agents. An adjuvant may be a water-in-oil emulsion in which antigen solution is emulsified in mineral oil (for example, Freund's incomplete adjuvant), sometimes with the inclusion of killed mycobacteria (Freund's complete adjuvant) to further enhance antigenicity. In one example, the adjuvant is a mixture of stabilizing detergents, micelle-forming agent, and oil available under the name PROVAX® (IDEC Pharmaceuticals, San Diego, Calif.). One of skill in the art can select a suitable adjuvant or combination of adjuvants to be included in the compositions disclosed herein or administered to a subject in combination with the compositions disclosed herein. Molecules with immune stimulant or adjuvant effects, include, without limitation: TLR3 agonists such as Poly(I:C) or Poly-ICLC; TLR 4 agonists such as LPS or monophosphoryl lipid derivatives; TLR 5 agonists such as flagellin derivatives; TLR 7/8 agonists such as imiquimod or R848; TLR 9 agonists such as CpG sequences; Stimulator of Interferon Genes (STING) pathway agonists such as ADU-S100; stimulatory neuroimmune mediators such as calcitonin gene-related peptide (CGRP); neurokinin 1 (NK1) receptor agonists such as Hemokinin 1 and Substance P; saponin related adjuvants such as QS-21 (Quillaja saponaria); purinoergic receptor agonists such as ATP; or oil-in-water emulsion adjuvants such as MF59.

A “polymer composition” is a composition comprising one or more polymers. As a class, “polymers” includes, without limitation, homopolymers, heteropolymers, co-polymers, block polymers, block co-polymers and can be both natural and/or synthetic. Homopolymers contain one type of building block, or monomer, whereas copolymers contain more than one type of monomer. The term “(co)polymer” and like terms refer to either homopolymers or copolymers. A polymer may have any shape for the chain making up the backbone of the polymer, including, without limitation: linear, branched, networked, star, brush, comb, or dendritic shapes. Molecular weight (MW) or molecular mass may refer to either number average molecular weight (Mn) or weight average molecular weight (Mw) unless specified.

A polymer “comprises” or is “derived from” a stated monomer if that monomer is incorporated into the polymer. Thus, the incorporated monomer (monomer residue) that the polymer comprises is not the same as the monomer prior to incorporation into a polymer, in that at the very least, certain groups/moieties are missing and/or modified when incorporated into the polymer backbone. A polymer is said to comprise a specific type of linkage if that linkage is present in the polymer, such as, without limitation: ester, amide, carbonyl, ether, thioester, thioether, disulfide, sulfonyl, amine, carbonyl, or carbamate bonds. The polymer may be a homopolymer, a copolymer, and/or a polymeric blend. A polymer is bioerodible if it degrades essentially completely in vivo in less than two years, less than one year, less than six months, less than three months, or less than one month.

By “biocompatible”, it is meant that a compound, composition, device, etc. is essentially, practically (for its intended use) and/or substantially non-toxic, non-injurious or non-inhibiting or non-inhibitory to cells, tissues, organs, and/or organ systems that would come into contact with the compound, composition, device, etc.

A bioerodible polymer also may be prepared from and therefore may comprise, without limitation, one or more of the following monomer residues: glycolide, lactide, caprolactone, dioxanone, and trimethylene carbonate. In general, useful (co)polymers may comprise monomers derived from alpha-hydroxy acids including polylactide, poly(lactide-co-glycolide), poly(l-lactide-co-caprolactone), polyglycolic acid, poly(dl-lactide-co-glycolide), and poly(l-lactide-co-dl-lactide); monomers derived from esters including polyhydroxybutyrate, polyhydroxyvalerate, polydioxanone, and polyglactin; monomers derived from lactones including polycaprolactone; monomers derived from carbonates including polycarbonate, polyglyconate, poly(glycolide-co-trimethylene carbonate), and poly(glycolide-co-trimethylene carbonate-co-dioxanone); monomers joined through urethane linkages, including poly(ester urethane) urea (PEUU), poly(ether ester urethane)urea (PEEUU), poly(ester carbonate)urethane urea (PECUU), or poly(carbonate)urethane urea (PCUU). Examples of suitable polymers may include, but are not limited to, polyacrylate, polymethacrylates, polyacrylamides, polymethacrylamides, polypeptides, polystyrenes, polyethylene oxides (PEO), poly(organo)phosphazenes, poly-l-lysine, polyethyleneimine (PEI), poly-d,l-lactide-co-glycolide (PLGA), and poly(alkylcyanoacrylate).

Polymer functionality may, for example, be linear or branched, and may include a poly(ethylene glycol) (PEG), a PEG-like group, an amine-bearing group (including primary, secondary, tertiary amine groups), a cationic group (which may generally be any cationic group—examples include a quaternary ammonium group, a guanidine group (guanidinium group), a phosphonium group or a sulfonium group), a dimethylsulfoxide-like (DMSO-like) group including methylsulfinyl-terminated alkyl groups, such as methylsulfinyl-terminated C₁-C₆ alkyl groups, or a zwitterionic group, such as a betaine, a reactive group for modification of polymer with, for example, small molecules (including, for example, dyes and targeting agents), a polymer, a biomolecule, or a biologically-active agent, such as a therapeutic agent, for example a peptide such as a cytokine or a growth factor, a polysaccharide, an oligonucleotide, a biologic active agent, or a small-molecule active agent.

The pharmaceutical compositions or dosage forms described herein may comprise, for delivery with the described replication-defective adenovirus infectious units, an adjuvant. The pharmaceutical compositions or dosage forms may also comprise, for delivery with the described replication-defective adenovirus infectious units, an additional therapeutic agent.

One or more of the disclosed adenoviral vectors encoding the immunogens, are administered to a subject by any of the routes normally used for introducing a composition into a subject. Methods of administration include, but are not limited to, intradermal, intramuscular, intraperitoneal, parenteral, intravenous, subcutaneous, vaginal, rectal, intranasal, inhalation, or oral. Parenteral administration, such as subcutaneous, intravenous or intramuscular administration, is generally achieved by injection. Injectables can be prepared in conventional forms, either as liquid solutions or suspensions, solid forms suitable for solution or suspension in liquid prior to injection, or as emulsions. Injection solutions and suspensions can be prepared from sterile powders, granules, and tablets of the kind previously described. Administration can be systemic or local.

The pharmaceutical compositions described herein comprise the described replication-defective adenovirus infectious unit and at least one carrier in a suitable dosage-form for delivery via a suitable route. The pharmaceutical dosage form may be a nasal spray device for delivery of, e.g., droplets or aerosol, for intranasal delivery, for example, as are broadly-known in the pharmacological and drug delivery arts. The nasal spray dosage form may comprises a container, the adenoviral infectious units contained within the container in a pharmaceutically-acceptable composition, optionally including an adjuvant, and a spray actuator for producing a spray or aerosol of the composition. Alternatively, the pharmaceutically-acceptable composition comprising the adenoviral infectious units may be in the form of a powder that is contained within an inhaler or a metered dose inhaler for delivery of one or more doses of the pharmaceutically-acceptable composition comprising the adenoviral infectious units by inhalation through the nose or mouth.

Alternatively, the replication-defective adenovirus infectious unit may be administered intracutaneously or subcutaneously by a microneedle array, for example as described herein.

The replication-defective adenovirus infectious unit may be administered with a pharmaceutically acceptable carrier. Choice of pharmaceutically acceptable carriers may be determined, at least in part, by the particular composition(s) being administered, as well as by the particular method used to administer the composition. Accordingly, there is a wide variety of suitable formulations of pharmaceutical compositions of the present disclosure. The replication-defective adenovirus infectious unit may be administered with an adjuvant. The adjuvant may be a cyclic dinucleotide, such as, but not limited to, 2′3′-cGAMP (cyclic [G(2′,5′)pA(3′,5′)p]). However, any adjuvant can be utilized, and efficacy thereof may be determined empirically. The immunogenic compositions may be conveniently presented in unit dosage form and prepared using conventional pharmaceutical techniques. Such techniques include the step of bringing into association the active ingredient and the pharmaceutical carrier(s) or excipient(s). In general, the formulations are prepared by uniformly and intimately bringing into association the active ingredient with appropriate carriers.

The formulations may be presented in unit-dose or multi-dose containers, for example, sealed ampules, vials, microneedle injection devices, syringes, etc., and optionally and where appropriate, may be stored in a freeze-dried (lyophilized) condition, requiring only the addition of a sterile liquid carrier, for example, water for injections, immediately prior to use. Extemporaneous injection solutions and suspensions may be prepared from sterile powders, granules and tablets commonly used by one of ordinary skill in the art.

Provided herein are methods of eliciting an immune response in a subject by administering to the subject a replication-defective adenovirus infectious unit, an immunogen, or a vector encoding the immunogen, as disclosed herein. In a particular example, the subject is a human. The replication-defective adenovirus infectious unit encoding the immunogen, is used, for example, to produce an immune response that prevents or inhibits infection with a coronavirus, e.g., a SARS-CoV-2.

In some examples, the method further includes selecting a subject in need of enhanced immunity to a coronavirus, e.g., a SARS-CoV-2. Subjects in need of enhanced immunity to coronavirus, e.g., a SARS-CoV-2 include subjects who are at risk of coronavirus, e.g., a SARS-CoV-2 infection, subjects who have been exposed to one or more coronavirus, e.g., a SARS-CoV-2, and subjects who have previously been vaccinated with a coronavirus, e.g., a SARS-CoV-2 vaccine. Additional factors that contribute to risk of infection with coronavirus, e.g., a SARS-CoV-2 include the characteristics of the location, presence of coronavirus, e.g., a SARS-CoV-2 in the area, and lack of preventive measures. The subject can be female, such as a human of child-bearing age.

Preparations for administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

As shown herein, benefits are seen from delivery of the replication-defective adenovirus infectious unit mucosally, e.g., intranasally or nasopharyngeally. Intracutaneous delivery, e.g., via a dissolvable microneedle array, also enables efficient and safe drug and vaccine delivery to the skin and mucosal surfaces. However, inefficient drug delivery can result from the homogenous nature of conventional microneedle array fabrication. Although the drugs or other cargo that is to be delivered to the patient are generally incorporated into the entire microneedle array matrix, in practice only the microneedles enter the skin and therefore, only cargo contained in the volume of the individual needles is deliverable. Accordingly, the vast majority of the drugs or other cargo that is localized in the non-needle components (e.g., the supporting structure of the array) is never delivered to the patient and is generally discarded as waste. As such, as an alternative delivery device, a fully-dissolvable microneedle array substrate and unique microneedle geometries as described I the examples below, may be utilized, that enable effective delivery of the vectors encoding the disclosed immunogens. This technology can also uniquely enable the simultaneous co-delivery of multiple chemically distinct agents for polyfunctional drug delivery. Examples of the utility of these devices include, for example, (1) simultaneous delivery of the disclosed replication-defective adenovirus infectious units and optionally adjuvants to generate an immune response relevant to coronavirus disease prevention, and (2) localized skin delivery.

A dissolvable microneedle array for transdermal insertion, e.g., local cutaneous delivery, into a subject may be provided for promoting an immune response against a coronavirus in a subject in need thereof. The array includes a base portion and a plurality of microneedles extending from the base portion and containing a disclosed immunogen, or a vector encoding the immunogen, and optionally at least one adjuvant.

The plurality of microneedles may be pre-formed to have a shape that comprises a first cross-sectional dimension at a top portion, a second cross-sectional dimension at a bottom portion, and a third cross-sectional dimension at an intermediate portion, wherein the intermediate portion is located between the top portion and the bottom portion, and the third cross-sectional dimension is greater than the first and second cross-sectional dimensions. Each microneedle may comprise a plurality of layers of dissoluble biocompatible material, such as, but not limited to carboxymethylcellulose.

A fabrication technology may be utilized that results in various active components to be incorporated into the needle tips, see U.S. Published Patent Application No. US 2016/0271381 A1, which is incorporated herein by reference. Thus, by localizing the active components in this manner, the remainder of the microneedle array volume includes less expensive matrix material that is non-active and generally regarded as safe. The net result is greatly improved efficiency of drug delivery based on (1) reduced waste of non-deliverable active components incorporated into the non-needle portions of the microneedle array, and (2) higher drug concentration in the skin penetrating needle tips.

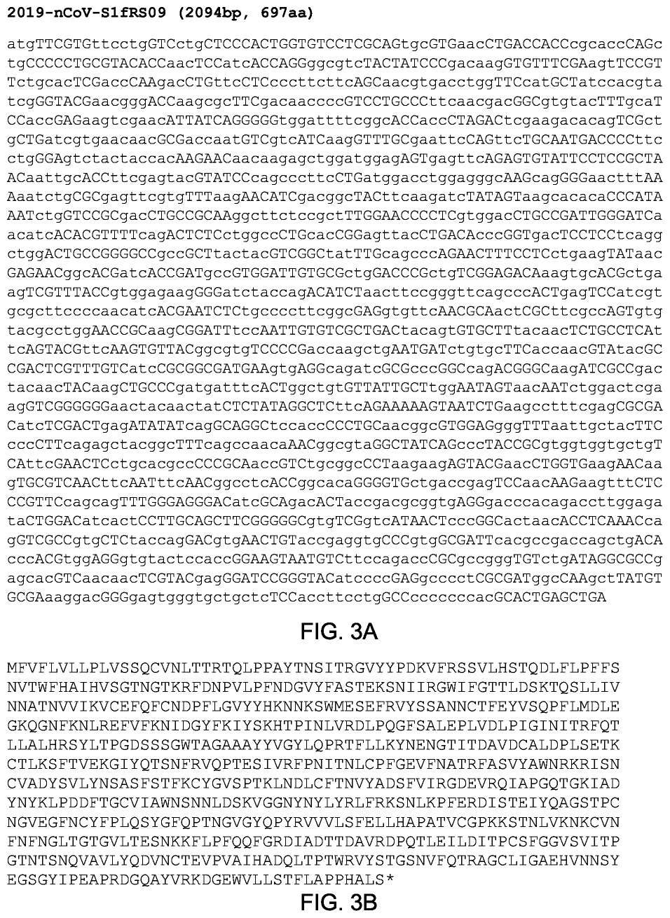

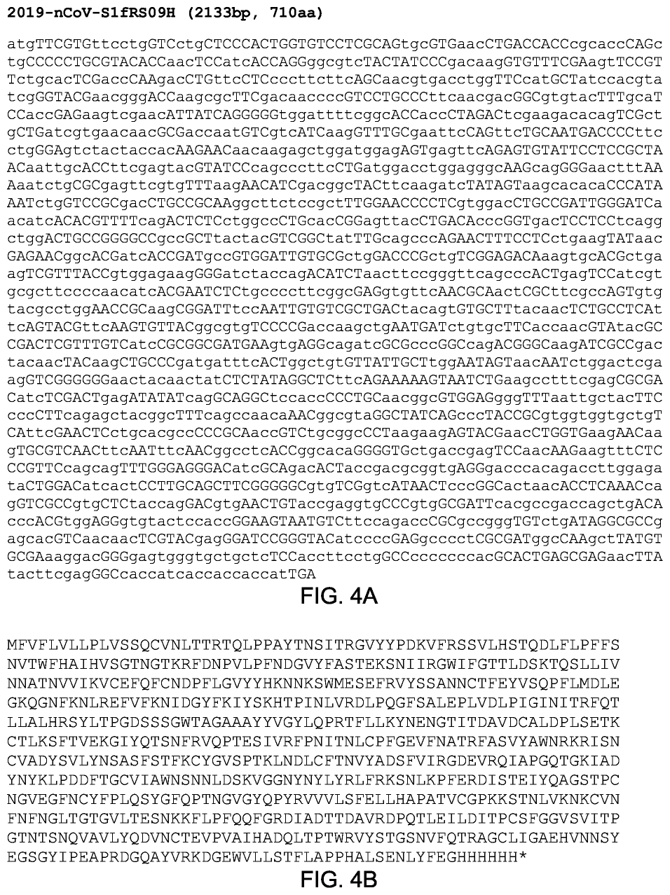

Thus, the active component may be concentrated in the microneedle tips of the respective arrays. In contrast to conventional microneedle arrays, the active component is not present at even concentration throughout the microneedle array since there is little or no active component present in the supporting base structure. In addition, as shown, for example, in FIGS. 3A, 3B, 4A, and 4B of U.S. Published Patent Application No. US 2016/0271381 A1, which is incorporated herein by reference, not only is there little or no active component in the supporting structures, the location of the active component is concentrated in the upper half of the individual microneedles in the array. The active component may be concentrated in the upper half of the individual microneedles. The active component may be concentrated in the tip of the microneedle, with the tip being defined by an area of the microneedle that extends from a base portion in a narrowing and/or tapered manner. The base portion, in turn, extends from the supporting structure of the array.

As noted above, individual microneedles may comprise active components only in the upper half of the microneedle. Individual microneedles may comprise active components only in the tips or in a narrowing portion near the tip of the microneedle. Individual needles may comprise active components throughout the entire microneedle portion that extends from the supporting structure, see U.S. Published Patent Application No. US 2016/0271381 A1, which is incorporated herein by reference.

The disclosed immunogens also may be delivered as disclosed in PCT Application No. PCT/US2016/057363, which is incorporated herein by reference. This PCT application disclosed microneedle arrays that can be configured to penetrate the stratum corneum to deliver their cargo (e.g., biologics or bioactive components) to the epidermis and/or dermis, while minimizing pain and bleeding by preventing penetration to deeper layers that may contain nerve endings and vessels. Pyramidal CMC-microneedles effectively penetrated the stratum corneum, epidermis, and dermis of living human skin, and thus can be used for cutaneous delivery. Thus, the microneedle array may include pyramidal CMC-microneedles.

To construct the microneedle arrays, a base material may be used to form portions of each microneedle that have bioactive components and portions that do not. As discussed above, each microneedle can comprise bioactive components only in the microneedles, or in some embodiments, only in the upper half of the microneedles, or in other embodiments, only in a portion of the microneedle that tapers near the tip. Thus, to control the delivery of the bioactive component(s) and to control the cost of the microneedle arrays, each microneedle preferably has a portion with a bioactive component (immunogen and/or adjuvant) and a portion without a bioactive component. In the embodiments described herein, the portion without the bioactive component includes the supporting structure of the microneedle array and, in some embodiments, a base portion (e.g., a lower half) of each microneedle in the array.

Various materials may be used as the base material for the microneedle arrays. The structural substrates of biodegradable solid microneedles may include poly(lactic-co-glycolic acid) (PLGA) or carboxymethylcellulose (CMC) based formulations; however, other bases can be used. For delivery of recombinant adenovirus particles, trehalose may be included in the portions of the microneedles comprising the recombinant adenovirus particles.

CMC may be preferable to PLGA as the base material of the microneedle arrays described herein. The PLGA based devices can limit drug delivery and vaccine applications due to the relatively high temperature (e.g., 135 degrees Celsius or higher) and vacuum required for fabrication. In contrast, a CMC-based matrix can be formed at room temperature in a simple spin-casting and drying process, making CMC-microneedle arrays more desirable for incorporation of sensitive biologics, peptides, proteins, nucleic acids, and other various bioactive components.

CMC-hydrogel may be prepared from low viscosity sodium salt of CMC with or without active components (as described below) in sterile dH₂O. In an exemplary embodiment, CMC can be mixed with sterile distilled water (dH₂O) and with the active components to achieve about 25 wt % CMC concentration. The resulting mixture can be stirred to homogeneity and equilibrated at about 4° C. for 24 hours. During this period, the CMC and any other components may be hydrated and a hydrogel can be formed. The hydrogel may be degassed in a vacuum for about an hour and centrifuged at about 20,000 g for an hour to remove residual micro-sized air bubbles that might interfere with a spin casting/drying process of the CMC-microneedle arrays. The dry matter content of the hydrogel can be tested by drying a fraction (10 g) of it at 85° C. for about 72 hours. The ready-to-use CMC-hydrogel is desirably stored at about 4° C. until use.

Active components, such as a disclosed immunogen or a vector encoding the immunogen, and optionally an adjuvant, may be incorporated in a hydrogel of CMC at a relatively high (20-30%) CMC-dry biologics weight ratio before the spin-casting process. Arrays can be spin-cast at room temperature, making the process compatible with the functional stability of a structurally broad range of bioactive components. Since the master and production molds can be reusable for a large number of fabrication cycles, the fabrication costs can be greatly reduced. The resulting dehydrated CMC-microneedle arrays are generally stable at room temperature or slightly lower temperatures (such as about 4° C.), and preserve the activity of the incorporated biologics, facilitating easy, low cost storage and distribution.

In one example, the surface of the production molds may be covered with about 50 μl (for molds with 11 mm diameter) of CMC-hydrogel and spin-casted by centrifugation at 2,500 g for about 5 minutes. After the initial CMC-hydrogel layer, another 50 μl CMC-hydrogel can be layered over the mold and centrifuged for about 4 hours at 2,500 g. At the end of a drying process, the CMC-microneedle arrays are separated from the molds, trimmed off from excess material at the edges, collected and stored at about 4° C. The production molds may be cleaned and reused for further casting of microneedle arrays.

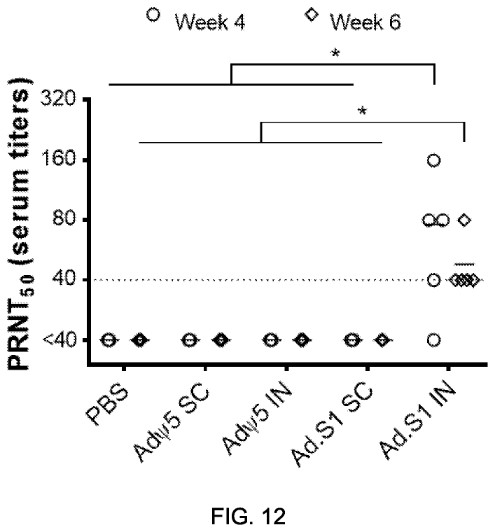

CMC-solids may be formed with layers that do not contain active components and layers that contain active components. FIGS. 11A-D of PCT Application No. PCT/US2016/057363, incorporated herein by reference) illustrate CMC-solids with different shapes (FIGS. 11A and 11B of PCT Application No. PCT/US2016/057363) and embedded active cargos on an upper layer which becomes, after micromilling, the portions of the microneedle with the active components. FIGS. 12A and 12B of PCT/US2016/057363, also illustrate CMC-solids with different shapes, with FIG. 12B showing a square shape and FIG. 12B showing a rectangular shape. Both CMC solids can be milled to dimensions for further processing as described herein. It should be understood that the geometries are not intended to be limiting. Any geometry can be used with the immunogens and vectors disclosed herein.

United States Patent Publication Nos. 2011/0098651; 2014/0350472; 2015/0126923, 2016/0271381, and U.S. Pat. No. 8,834,423, describe certain exemplary microneedle arrays and methods of making and using microneedle arrays. As an example, apparatuses and methods are described for fabricating dissolvable microneedle arrays using master molds formed by micromilling techniques. For example, microneedle arrays can be fabricated based on a mastermold (positive) to production mold (negative) to array (positive) methodology. Micromilling technology can be used to generate various micro-scale geometries on virtually any type of material, including metal, polymer, and ceramic parts. Micromilled mastermolds of various shapes and configurations can be effectively used to generate multiple identical female production molds. The female production molds can then be used to microcast various microneedle arrays. Direct micromilling of mastermolds can replace other exemplary microneedle array production methods that involve expensive, complex and equipment-sensitive SU-8 based lithography or laser etching techniques, which are conventionally used to create mastermolds for dissolvable needle arrays. In addition, as discussed below, micromilling can provide for the construction of more complex mastermold features than can conventional lithography and laser etching processes. Precision-micromilling systems can be used for fabricating a microneedle mastermold, using micro-scale (for example, as small as 10 μm (micrometers or microns)) milling tools within precision computer controlled miniature machine-tool platforms. The system can include a microscope to view the surface of the workpiece that is being cut by the micro-tool. The micro-tool can be rotated at ultra-high speeds (200,000 rpm) to cut the workpiece to create the desired shapes. Micromilling process can be used to create complex geometric features with many kinds of material, which are not possible using conventional lithographic or laser etching processes. Various types of tooling can be used in the micromilling process, including, for example, carbide micro-tools or diamond tools.

Mastermolds can be micromilled from various materials, including, for example, Cirlex® (DuPont, Kapton® polyimide). Mastermolds can be used to fabricate flexible production molds from a suitable material, such as a silicone elastomer, e.g., SYLGARD® 184 (Dow Corning). The mastermold is desirably formed of a material that is capable of being reused so that a single mastermold can be repeatedly used to fabricate a large number of production molds. Similarly each production mold is desirably able to fabricate multiple microneedle arrays.

In one example, production molds are made from SYLGARD® 184 (Dow Corning), and are mixed at a 10:1 SYLGARD® to curing agent ratio. The mixture is degassed for about 10 minutes and poured over the mastermold to form an approximately 8 mm layer, subsequently degassed again for about 30 minutes and cured at 85° C. for 45 minutes. After cooling down to room temperature, the mastermold is separated from the cured silicone, and the silicone production mold is trimmed. From a single mastermold, a large number of production molds (e.g., 100 or more) can be produced with very little, if any, apparent deterioration of the Cirlex® or acrylic mastermolds.

In one example, to construct the microneedle arrays, a base material is used to form portions of each microneedle that have bioactive components and portions that do not. Of course, if desired, each microneedle can comprise only portions that contain bioactive components; however, to control the delivery of the bioactive component(s) and to control the cost of the microneedle arrays, each microneedle optionally is constructed such that a portion of the structure has a bioactive component and a portion does not include a bioactive component. Variations in the size, shape and number of the microneedles, and location of the bioactive component(s) in the microneedles, may be readily varied by varying the mastermold, or by varying the deposition and patterning of the materials used to produce the microarray.

Alternatively, the MNA may include microneedles that are not dissolvable, but that include the immunogen-containing composition coated thereon, or contained within a lumen or via thereof, which also allows for access to skin cells.

A large variety of materials useful for preparation of the microneedle array are available, along with variation in the location of such materials in the microarray. Precise positioning and layering of the materials during, e.g., spin casting, of the microneedle array will yield any desired structure.

The microneedle array, both base and needles, may be manufactured from a single carrier composition including a dissolvable composition and a bioactive agent, such as a reporter gene, such as the replication-defective adenovirus infectious unit for expressing a coronavirus, e.g., a SARS-CoV-2, spike protein-, and/or nucleoprotein protein-based immunogen comprising at least an immunogenic sequence of a spike S1 protein, and/or nucleoprotein protein. The “carrier composition” is one or more dissolvable and/or bioerodible compounds or compositions into which a bioactive agent is mixed, and in the context of the present disclosure forms a structure with physical parameters, and lack of negative effects on the bioactive agent as used herein, including sufficient safety to a patient, such that the carrier composition is useful as a component of the microneedles and microneedle arrays.

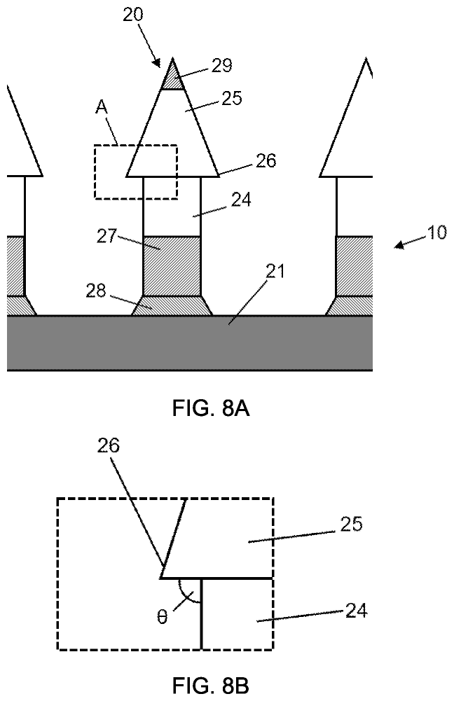

FIGS. 8A and 8B depict a microneedle array 10, in part, having a microneedle 20 and a backing layer 21. Additional, adjacent microneedles are partially depicted. The microneedle 20 comprises a stem 24 and a head 25. The head 25 comprises a ridge 26 extending beyond the peripheral boundary, e.g., circumference, of the stem 24, and as such, the head 25 may be described as undercut or barbed. The stem 24 may comprise a single material, which may be identical to the material of the head 25, or may be different than the material of the head 25. Although the stem 24 may be manufactured from a single material, it may comprise a dissolvable portion 27 that dissolves faster than the material of the head 25, e.g., within seconds under physiological conditions. The dissolvable portion 27 of the stem 24 may comprise a sugar or low molecular-weight dimer, oligomer, or polymer. In use, the microneedle 20 penetrates a patient's skin and the dissolvable portion rapidly-dissolves, either fully releasing the remainder of the microneedle in the patient's skin, or rendering that portion of the stem 24 sufficiently frangible, such that by pulling the backing layer 21 away from the patient's skin, and assisted by the undercutting of the head 25, the heads 25 of the microneedles, and any slower-dissolving portions of the stem 24, as compared to the dissolvable portion 27, will remain in the patient's skin. In embodiments of the microneedle array 10 where the stem 24 does not comprise a dissolvable portion 27, meaning the dissolvable portion 27 is the same material as the stem 24, the stem 24 still remains or is rendered frangible and may be retained in the patient's skin by dissolving and/or breaking of the stem 24. The head 25, and optionally the stem 24, may comprise a bioactive or therapeutic agent such as the immunogen described herein mixed into, co-deposited in the mold with, absorbed into, or adsorbed onto the microneedle. Depicted in FIG. 8A is a head 25 with a tip portion 29 in which a therapeutic agent is concentrated, e.g., by spin casting as described below.

In use, the microneedles 20 of the microneedle array 10 are pressed into the skin of a patient, and are left in place for a sufficient time to either inoculate the patient, or for the stems 24 and/or the dissolvable portions 27 of the microneedles to be dissolved or rendered sufficiently frangible so that removal of the backing layer would release the heads 25 of the microneedles 20 into the skin of the patient, such that the therapeutic agent(s), such as the immunogen, contained in and/or on the microneedles, is released into the skin of the patient. To prevent breaking off of the microneedles 20 on the patient's skin, prior to piercing of the skin with the microneedles 20, the base of the stem 24 of the microneedles may be reinforced, such as by use of a filleted base 28 as shown in FIG. 8A.

FIG. 8B depicts an enlargement of portion A, within the dashed rectangle as shown in FIG. 8A. Stem 24, head 25, and ridge 26 are depicted. FIG. 8B depicts the ridge 26 extending at an angle θ from the stem 24, where θ is 90°. In practice, θ may vary from 90°, and may range, from 20° to 100°, from 45° to 100°, from 60° to 95°, or 90°±2°, such as, without limitation, 75°, 76°, 77°, 78°, 79°, 80°, 81°, 82°, 83°, 84°, 85°, 86°, 87°, 88°, 89°, 90°, 91°, 92°, 93°, 94°, or 95°. It should be recognized that the undercutting of the head and the transition from the head to the stem may not be perfectly linear, and these angles represent an average or best fit of the relationship between the stem and the underside of the ridge. In aspects, the amount of undercutting will be dictated by the ability to manufacture such structures, for example, using the methods described herein, including the ability to remove the microneedles from the mold after spin-casting. As such, the angle of undercut may be 90°, or may approach 90°, such as 90°±1°, 90°±2°, 90°±3°, 90°±4°, or 90°±5°.

The microneedle array, and elements thereof are depicted in FIGS. 8A and 8B are having specific geometries, such as a cylindrical stem 24 and dissolvable portion 27, a conical head 25, a ridge 26 or undercut extending at a 90° angle (perpendicularly) from the stem 24, and a triangle-profiled filleted base. It would be understood that these geometries are merely exemplary and one of ordinary skill could adapt different shape profiles and still include the structure depicted in FIGS. 8A and 8B, while retaining the functional aspects of those structures.

Administration may be accomplished by single or multiple doses, e.g., a single vaccination or a prime-boost vaccination (homologous or heterologous). The dose administered to a subject in the context of the present disclosure may be sufficient to induce a beneficial therapeutic response in a subject over time, or to inhibit or prevent coronavirus, e.g., SARS-CoV-2 infection. The dose required may vary from subject-to-subject depending on the species, age, weight and general condition of the subject, the severity of the infection being treated, the particular immunogenic composition being used, and its mode of administration. An appropriate dose may be determined empirically.

The volume of administration may vary depending on the route of administration. By way of example, intramuscular injections may range from about 0.1 ml to about 1.0 ml. Appropriate volumes for different routes of administration may be determined empirically.

Repeated immunizations may be necessary to produce an immune response in a subject. When administered in multiple doses, the booster doses are administered at various time intervals, such as weeks or months to years. In other examples, there are one or more vectors encoding a disclosed immunogen are used as a booster following administration of one or more coronavirus, e.g., SARS-CoV-2, vaccines. In one example, a subject is administered a prime dose of a coronavirus, e.g., SARS-CoV-2, vaccine followed by at least one boost dose of an immunogen, or a vector encoding the immunogen, as disclosed herein. In alternative examples, the immunogen, or the vector encoding the immunogen is administered first, followed by a booster administration of another coronavirus, e.g., SARS-CoV-2, vaccine, such as an inactivated coronavirus, e.g., SARS-CoV-2, vaccine.

A prime boost strategy may be utilized. For example, a boost dose is administered about 14, 30, 60, 90, or more days after administration of the prime dose. Additional boosters can be administered at subsequent time points, if determined to be necessary or beneficial. Immunization protocols (such as amount of immunogen, number of doses and timing of administration) may be determined experimentally, for example by using animal models (such as mice or non-human primates), followed by clinical testing in humans.

Initial injections or doses may range from about 1 μg to about 1 mg, with some embodiments having a range of about 10 μg to about 800 μg, or from about 25 μg to about 500 μg. Following an initial administration of the immune stimulatory composition, subjects may receive one or several booster administrations, adequately spaced. Booster administrations may range from about 1 μg to about 1 mg, with other embodiments having a range of about 10 μg to about 750 μg, and still others a range of about 50 μg to about 500 μg. Periodic boosters at intervals of 1-5 years, for instance three years, may be desirable to maintain the desired levels of protective immunity.

Following immunization, the immune response may be assessed. For example, a biological sample may be obtained from the subject, and antibodies and/or reactive T cells specific for coronavirus, e.g., SARS-CoV-2, can be assessed.

The following examples are provided to illustrate certain particular features and/or embodiments. These examples should not be construed to limit the disclosure to the particular features or embodiments described.

### EXAMPLES

The following examples are intended to be illustrative and exemplary.

### Example 1

In addition to the intranasal route, the skin may be an attractive tissue target for vaccination, as it is readily accessible and contains a dense population of antigen-presenting and immune-accessory cells. Microneedle arrays (MNAs) are emerging as an effective tool for in situ engineering of the cutaneous microenvironment to enable diverse immunization strategies. Here, novel dissolving undercut MNAs and demonstrate their application for effective multicomponent cutaneous vaccination are presented. The MNAs are composed of micron-scale needles featuring pyramidal heads supported by undercut stem regions with filleted bases to ensure successful skin penetration and retention during application. Prior efforts to fabricate dissolving undercut microstructures were limited and required complex and lengthy processing and assembly steps. In the current study, we strategically combine three-dimensional (3D) laser lithography, an emerging micro-additive manufacturing method with unique geometric capabilities and nanoscale resolution, and micromolding with favorable materials. This approach enables reproducible production of dissolving MNAs with undercut microneedles that can be tip-loaded with multiple biocargos, such as antigen (ovalbumin) and adjuvant (Poly(I:C). The resulting MNAs fulfill the geometric (sharp tips and smooth edges) and mechanical-strength requirements for failure-free penetration of human and murine skin to simultaneously deliver multicomponent (antigen plus adjuvant) vaccines to the same cutaneous microenvironment. Cutaneous vaccination of mice using these MNAs induces more potent antigen-specific cellular and humoral immune responses than those elicited by traditional intramuscular injection. Together, the unique geometric features of these undercut MNAs and the associated manufacturing strategy, which is compatible with diverse drugs and biologics, could enable a broad range of non-cutaneous and cutaneous drug delivery applications, including multicomponent vaccination.

### Materials and Methods

Ovalbumin (OVA; #A5503), polyinosinic-polycytidylic acid sodium salt (Poly(I:C); #P1530), carboxymethylcellulose (CMC, 90 kDa MW), D-(+)-trehalose dihydrate, polyvinylpyrrolidone (PVP, 40 kDa MW), polyvinyl alcohol (PVA, 87-90% hydrolyzed, 30-70 kDa MW), Allura Red AC (R40 dye), doxorubicin, 4,4′,5,5′-tetramethylbenzidine (TMB) peroxidase substrate, carbonate-bicarbonate buffer (pH 9.6), and Tween20 were purchased from Sigma-Aldrich (St. Louis, Mo.). Polydimethylsiloxane (PDMS) SYLGARD® 184 and VeroWhiteplus-RGD835 UV-curable resin were obtained from Dow Corning (Midland, Mich.) and Stratasys (Eden Prairie, Minn.), respectively. Green fluorescent Degradex PLGA microspheres (10 μm diameter) were acquired from Phosphorex (Hopkinton, Mass.). Alexa 555-labeled OVA (Invitrogen), Alexa 680-labeled OVA (Invitrogen), Texas Red-labeled dextran (40 kDa MW; Invitrogen), Pierce Micro BCA Protein Assay Kit, SYBR Green EMSA nucleic acid stain, endotoxin-free HyClone Cell Culture Grade Water, RNase-free Ambion TE Buffer (pH 8.0), carboxyfluorescein succinimidyl ester (CFSE; Invitrogen), and DAPI were purchased from Thermo Fisher Scientific (Waltham, Mass.). Anti-OVA IgG1 (Cayman Chemical, Ann Arbor, Mich.), anti-OVA IgG2c (Chondrex, Redmond, Wash.), normal goat serum and biotinylated goat anti-mouse IgG1 and IgG2c secondary antibodies (Jackson ImmunoResearch, West Grove, Pa.), streptavidin-HRP (BD Biosciences, San Jose, Calif.), and OVA257-264 (SIINFEKL) peptide (Anaspec, Fremont, Calif.) were used forimmune assays.

### Fabrication of Dissolving Microneedle Arrays

Microneedle and array designs: The unique microneedle array (MNA) design utilized in this study is essentially as shown in FIGS. 8A-8B. This particular microneedle design consisted of a sharp-tipped pyramid head and an undercut stem portion with a filleted base. The microneedle was 750 μm in height with a 30° apex angle. The stem portion of the microneedle was 150 μm in width and extended from the bottom of a square pyramid head (250 μm×250 μm base area) to the backing layer of MNA with a 35 μm radius filleted connection. The fillet was specifically designed at the microneedle base to avoid sharp corners and associated mechanical stress concentration, considerably increasing microneedle strength performance during manufacturing processes and skin insertion (Bediz et al., “Dissolvable microneedle arrays for intradermal delivery of biologics: fabrication and application”, 2014, Pharm. Res., 31:117-135; Rad et al., “High-fidelity replication of thermoplastic microneedles with open microfluidic channels, 2017, Microsyst. Nanoeng., 3: 17034). The apex angle, width, and height of the microneedles were chosen based on skin anatomy and skin insertion mechanics to ensure failure-free penetration (Bediz et al.; Prausnitz, M. R. “Engineering Microneedle Patches for Vaccination and Drug Delivery to Skin”, 2017, Annu Rev Chem Biomol Eng; 8:177-200). Notably, this design introduces a novel undercut, or anchor feature, which improves skin retention during application, but still allows direct removal of MNAs from flexible production molds throughout the manufacturing process. The tip-to-tip distance between microneedles in the 5×5 arrays was 650 μm, and the size of MNA was 4.75 mm×4.75 mm. The array design (microneedle spacing) was based on solid mechanics considerations and skin insertion mechanics to avoid a “bed of nails” effect during skin penetration (Bediz et al.; Korkmaz et al., “Therapeutic intradermal delivery of tumor necrosis factor-alpha antibodies using tip-loaded dissolvable microneedle arrays”, 2015, Acta Biomater., 24:96-105; Verbaan et al., “Improved piercing of microneedle arrays in dermatomed human skin by an impact insertion method”, 2008, J. Control. Release, 128:80-88). The three-dimensional micro-additive manufacturing (3D-μAM) approach provides a simple, reproducible, and revolutionary means to produce the proposed unique MNA design from a 3D-CAD drawing, and allows individuals with no microfabrication expertise to easily create a broad range of MNA designs.

### Manufacturing Strategy

The manufacturing strategy used to fabricate dissolving MNAs with novel microneedle designs comprises a six-step approach that exploits μAM and micromolding to create dissolving undercut MNAs, while simultaneously achieving high-throughput fabrication: (1) 3D-CAD drawing of the MNA design; (2) direct production of a master MNA from the CAD drawing by 3D direct laser writing using a non-dissolvable resin (IP-S); (3) high-fidelity replication of master MNA with UV-curable resin (VeroWhite) by micromolding; (4) creation of MNA master molds that consist of multiple master MNA replicas on 3D-printed MNA holders; (5) manufacturing of elastomer (PDMS) MNA production molds by micromolding; and (6) fabrication of tip-loaded, dissolving MNAs with undercut microneedles incorporating a vaccine or other biocargo in a water-soluble biocompatible material (e.g., carboxymethylcellulose (CMC) and trehalose) through a spin-casting method. The last step of the process can be modified depending on the biocargo of interest, and typically involves spin-casting cargo (e.g., vaccine) into the tip of the PDMS production molds, followed by spin-casting a dissolvable hydrogel (e.g., CMC/trehalose) into the production molds to serve as the structural material. Notably, the master MNA, master molds consisting of multiple master MNA replicas, and elastomer production molds are reusable, reducing the fabrication costs for dissolving undercut MNAs. At each stage of the fabrication process, optical stereomicroscopy (ZEISS Stemi 2000-C microscope with Olympus OM-D E-M511 camera) was used to assess geometric integrity of the microneedles.

Fabrication of master MNA: The unique MNA geometry was designed in SolidWorks 2018 CAD software and directly created from the 3D-CAD drawings using 3D laser printing (Nanoscribe Photonic Professional, GT; Nanoscribe Struensee, Germany) with the photopolymeric resist IP-S. The Nanoscribe printing system was equipped with a laser generator, an optical cabinet, a Zeiss optical microscope attached to a lens to focus the laser beam, a Galvo mirror system to direct the laser-beam scanning, a piezoelectric stage for precise motion control, and software (Nanowrite) to execute 3D printing. The whole system was placed on an optical table to eliminate vibrations during the printing process.

To fabricate the master MNA, the CAD design was converted into ‘STL’ (StereoLithography) format. The STL file was loaded into the specialized software (DeScribe, Germany) for the Nanoscribe system to select the processing conditions (distance of slicing, hatching, and splitting). Finally, the STL file was converted into ‘GWL’ (General Writing Lithography) format and exported to the Nanowrite software to print the master MNA. The master MNA was fabricated using Galvo-scan mode in XY plane and piezo-scan mode in Z direction. The master MNA was split into 220 μm×220 μm×200 μm blocks within the working range and then stitched together. Laser power and writing speed were set to 100 mW and 6 cm/s, respectively. Minimum and maximum slicing distances of 0.3 μm and 0.5 μm, respectively, were used. The master MNA was then printed through two-photon polymerization of the IP-S photoresist by a femtosecond pulsed laser at a wavelength of 750 nm using a unique deep-in-liquid mode with a 25× NA0.8 objective in Shell and Scaffold mode. After printing, the master MNA was developed in the photoresist solvent propylene glycol monomethyl ether acetate (PGMEA) for 30 min, followed by a 5 min isopropyl alcohol rinse. The master MNA was then air-dried and placed under UV light (365 nm, 16 mW/cm² intensity) for 30 min to further crosslink the body to make the master MNA structure strong.

Replication of Master MNA: A two-stage micromolding method was used to replicate the master MNA with high-fidelity using a UV-curable resin. First, an elastomer mold, which is a negative mold of the master MNA, was manufactured from polydimethylsiloxane (PDMS) by soft-lithography. Elastomer molding with PDMS is a well-established technique for rapid, accurate, and reproducible replication of high-fidelity micron-scale structures (Losic et al., “Rapid fabrication of micro- and nanoscale patterns by replica molding from diatom biosilica”, 2007, Adv. Fund. Mater., 17:2439-2446; Gates et al., “Replication of vertical features smaller than 2 nm by soft lithography”, 2003, J. Am. Chem. Soc., 125:14986-14987). Briefly, the master MNA was mounted in a petri-dish with a diameter of 5 cm, and PDMS was prepared using a two-component curable silicone elastomer, SYLGARD® 184 (10:1 base-to-curing agent). The PDMS was poured over the master MNA mounted in the petri-dish and degassed for 15 min. Next, the master MNA with degassed PDMS was cured at 70° C. for 1 h. The cured PDMS was cooled to room temperature for 5 min and then separated from the master MNA to obtain the negative PDMS mold.

The second processing step used the negative PDMS mold to fabricate positive master MNA replicas from a UV-curable resin (VeroWhiteplus-RGD835). For each PDMS mold, 20 μL of liquid resin was poured onto the molds, and then the molds were centrifuged (4500 RPM at 20° C. for 1 min; Thermo Fisher Scientific Sorvall Legend XTR centrifuge with Swinging Bucket Rotor TX-750) to fill the microneedle-shaped wells with resin. The resin was then treated under UV light (365 nm) with 21.7 mW/cm² intensity for 5 min from both the top and bottom to cure the base and the microneedle tips. To ensure the backing layers of the master MNA replicas were flat, an additional 50 μL of UV-curable resin, which exceeded the remaining volume available, was deposited onto the PDMS mold. A glass slide was placed on top of the mold to get rid of the excess resin, thereby creating a uniform flat surface at the base. The liquid resin was then cured from the top side for 5 min and demolded to obtain a replica of the master MNA.

Creation of MNA master molds and production molds: To improve productivity of the manufacturing process for dissolving MNAs, the MNA master molds were created by assembling six master MNA replicas onto MNA holders fabricated by Stratasys® from a non-dissolvable photo-polymer (VeroWhite) using a high-resolution Polyjet 3D printing system (Objet Connex 500 multi-material). A 3D model of the MNA holder was created using SolidWorks 2018 CAD software and then converted into the ‘STL’ (StereoLithography) file format. Subsequently, the specialized software (Objet Studio) sliced this 3D model into 2D cross-sectional layers, creating a computer file that was sent to the 3D printer system at Stratasys. Channels in the 3D printed MNA holder were designed to serve as pockets in the MNA production molds to assist as reservoirs for both the bioactive cargo (e.g., vaccine) and the structural hydrogel material of dissolving MNAs during the spin-casting process. The MNA master molds were baked at 80° C. overnight in a vacuum oven to facilitate effective molding of elastomer MNA production molds. Subsequently, MNA production molds that included microneedle-shaped wells for six MNAs were fabricated from PDMS as described for replication of the master MNA. Notably, a single MNA master mold can be used repeatedly to fabricate multiple PDMS production molds.

Production of dissolving MNAs: Dissolving MNAs with novel undercut microneedles tip-loaded with multicomponent vaccines (OVA antigen ±Poly(I:C) adjuvant) were manufactured through a spin-casting technique with centrifugation at room temperature. First, 5 μL of an aqueous solution of OVA (25 mg/mL) was dispensed to each MNA reservoir on the PDMS production molds, and production molds were centrifuged (1 min at 4500 rpm) to fill the microneedle-shaped cavities. Excess OVA solution within the reservoir was then recovered, and production molds were centrifuged (30 min at 4500 rpm) to ensure that dry OVA cargo was located at the tip portion of the microneedle-shaped cavities in the production molds. For MNAs integrating OVA and Poly(I:C), the aforementioned process was repeated with 5 μL of an aqueous solution of Poly(I:C) (62.5 mg/mL). The final MNAs used for cutaneous vaccination experiments included 10 μg OVA and 25 μg Poly(I:C) per MNA. The tip-loading process and recovery of excess biocargo is depicted in FIG. 8.

After loading vaccine biocargo at the tips of microneedles, the MNA structural biomaterial was prepared by dissolving a 70:30 mixture of sodium carboxymethylcellulose (CMC) and D-(+)-trehalose dihydrate in endotoxin-free water at a total solute concentration of 30% w/w. The resulting CMC/trehalose hydrogel was loaded onto each MNA in the PDMS production molds (40 μL each) to fill the remaining volume of the microneedles and to form the MNA backing layer. Hydrogel-loaded production molds were centrifuged (5 h at 4500 rpm) to obtain the final dissolving undercut MNAs for cutaneous vaccination experiments. MNAs were then removed from production molds with tweezers, or forceps, by pulling two diagonal corners of the MNA base away from the mold. To demonstrate the broader material capabilities of our manufacturing strategy for dissolving MNAs with undercut microneedles, MNAs were also fabricated using a 40% w/w hydrogel with a 60:40 mixture of polyvinylpyrrolidone (PVP) and polyvinyl alcohol (PVA) through the spin-casting method.

### Quantification of Antigen and Adjuvant Loading

Microneedles were dissolved in TE Buffer, and concentrations of OVA and Poly(I:C) were measured using a Micro BCA protein assay and SYBR Green nucleic acid assay, respectively. Loading error, defined as the difference between measured and theoretical amounts of biocargo in microneedles as a percentage of the theoretical amount, was calculated. To determine loading efficiency, excess biocargo recovered from the MNA production mold reservoir after loading and prior to drying (FIG. 8 (C)) was quantified. Loading efficiency=[biocargo in microneedles/(biocargo loaded to mold−excess biocargo recovered)]×100%. Results are reported as mean±SD (N=6).

### Cutaneous Vaccine Delivery to Human Skin Explants Using MNAs

Preparation of ex vivo human skin explants: Human skin explants were prepared as described previously (Morelli et al., “CD4⁺ T cell responses elicited by different subsets of human skin migratory dendritic cells”, 2005, J. Immunol., 175:7905-7915). Briefly, normal human skin from deidentified healthy donors undergoing plastic surgery was acquired through the Pitt Biospecimen Core and used according to University of Pittsburgh Medical Center guidelines. Tissue was rinsed in 70% ethanol and then in phosphate-buffered saline (PBS). Human skin explants (approximately 1 mm thick) were harvested using a Silver's miniature skin graft knife (Padgett, Integra Miltex, Plainsboro, N.J.), and then cut into 20 mm×20 mm square pieces. The resulting human skin samples comprised epidermis and a thin layer of underlying dermis.

Imagining Analysis: To evaluate undercut MNA-directed intradermal biocargo (e.g., vaccine) delivery to living human skin explants, several imaging analyses were performed. Tip-loaded MNAs incorporating a red cargo (Allura Red R40 dye) were fabricated using the manufacturing strategy described above. Prior to application of MNAs to human skin explants, MNAs were imaged using an optical stereomicroscope. Subsequently, MNAs were applied to human skin explants and removed after 10 min. An optical stereomicroscope was then used to image the patterns of colored biocargo deposited from MNAs into the human skin. Remaining MNA materials after application were also imaged. For further qualitative assessment of MNA-directed intradermal vaccine delivery to human skin, MNAs containing both Alexa555-labeled OVA and Alexa488-labeled Poly(I:C) were fabricated, applied to human skin explants for 10 min, and removed. Targeted areas of the human skin explants were fixed in 2% paraformaldehyde, cryopreserved with 30% sucrose solution, flash frozen in optimum cutting temperature (OCT) compound, and cryo-sectioned into 10 μm thick sections. Human skin cross-sections were counter-stained with a fluorescent nuclear dye (DAPI) and imaged using a bright-field and epifluorescence microscope (Nikon Eclipse E800) to detect Alexa555-OVA and Alexa488-Poly(I:C), with bright-field images taken to better visualize the stratum corneum breaching.

### MNA-Directed Skin Immunization In Vivo

Mice: Female C57BL/6J mice were purchased from The Jackson Laboratory (Bar Harbor, Me.) and used at 8-10 weeks of age. Mice were maintained under specific pathogen-free conditions at the University of Pittsburgh, and all experiments were conducted in accordance with the institutional animal care and use committee (IACUC) guidelines.

Quantification of antigen and adjuvant delivery with MNAs: OVA+Poly(I:C) MNAs were applied to murine abdominal skin for 5, 10, or 20 min, and then remaining MNAs were removed and dissolved in TE Buffer. Concentrations of OVA and Poly(I:C) were measured using a Micro BCA protein assay and SYBR Green nucleic acid assay, respectively. Quantities of OVA and Poly(I:C) delivered to skin were calculated by subtracting the amount remaining from the mean amount loaded, and delivery is reported as percentage of the initial amount loaded (mean±SD, N=6).

In vivo IVIS imaging: In vivo intradermal vaccine delivery with dissolving undercut MNAs was demonstrated on a C57BL/6J mouse. Tip-loaded CMC/trehalose MNAs integrating both Alexa555-labeled OVA and Alexa488-labeled Poly(I:C) were created using the manufacturing strategy described above, applied to the abdomen of an anesthetized mouse for 10 min, and then removed. Fluorescent OVA+Poly(I:C) MNAs were imaged before and after in vivo application by optical stereomicroscopy and epifluorescence microscopy. To show delivery of the fluorescent multicomponent vaccine, the mouse was imaged with an IVIS 200 in vivo imaging system (PerkinElmer, Waltham, Mass.), using the corresponding filters to detect Alexa488-Poly(I:C) and Alexa555-OVA at the MNA application site. Images were then post-processed using Living Image software (Perkin Elmer).

Cell-mediated and humoral immune responses: Mice were immunized by application of 10 μg OVA±25 μg Poly(I:C) MNAs to the right and left sides of abdomen (two MNAs per mouse) or by two intramuscular injections of 10 μg OVA in PBS into the hindlimb gastrocnemius muscles. Control mice were left untreated (i.e., naïve), or treated with blank MNAs (without antigen or adjuvant). Cutaneous or intramuscular immunizations were repeated 7 days later. In vivo OVA-specific cytotoxic T-cell activity and OVA-specific antibody responses were evaluated 5 days after the second immunization (booster dose) using well-established techniques (Morelli et al.; Condon et al., “DNA-based immunization by in vivo transfection of dendritic cells”, 1996, Nat. Med., 2: 1122-1128).

For OVA-specific antibody responses, blood was collected from anesthetized mice at the time of sacrifice by cardiac puncture, and serum was isolated using BD Microtainer serum separator tubes (BD Biosciences, San Jose, Calif.). OVA-specific IgG1 and IgG2c antibodies in serum were measured by indirect ELISAs. Costar EIA/RIA plates (Corning Inc., Corning, N.Y.) were coated with OVA (100 μg/mL in 0.5 M carbonate-bicarbonate buffer) by overnight incubation at 4° C. Plates were washed (3×) with 0.05% Tween20 in PBS, and blocked with 1% goat serum in PBS for 1 h at 37° C. Serum samples and standards (anti-OVA IgG1 or anti-OVA IgG2c) were diluted with 1% goat serum, added to plates, and incubated 2 h at 37° C. After washing (3×), plates were incubated for 1 h at 37° C. with biotinylated secondary antibodies (goat anti-mouse IgG1 or IgG2c, 1:20,000 in 1% goat serum). Plates were then washed (3×) and incubated for 30 min with streptavidin-HRP (1:1000 in 1% goat serum). Plates were washed (3×) again and incubated at room temperature with TMB peroxidase substrate for 2-3 min, and the reaction quenched with 1.0 M H2504. For all ELISAs, absorbance at 450 nm (0D450) was read with a SpectraMax 340PC plate reader (Molecular Devices, Sunnyvale, Calif.), and serum concentrations calculated from standard curves.

To assess OVA-specific cytotoxic T-cell (CTL) activity, splenocytes from naïve mice were pulsed with 2 μg/mL OVA257-264 (SIINFEKL) peptide, or left unpulsed for 1 h. Antigen pulsed splenocytes were washed and stained with high concentration CFSE (10 μM), while unpulsed splenocytes were labeled with low concentration CFSE (1 μM) for 15 min at 37° C. A 1:1 mixture of pulsed target cells and unpulsed control cells (107 each) was intravenously (IV) injected into immunized and naïve mice. Twenty hours after adoptive transfer, spleens of mice were isolated, and killing of target cells was evaluated by comparison of the antigen pulsed and unpulsed populations by flow cytometry to quantify OVA-specific killing of the high CFSE labeled SIINFEKL-pulsed targets. Specific lysis was calculated and expressed as a percentage of maximum lysis as: % Lysis={1−[(mean CFSE^{low}/CFSE^{high} ratio from naïve mice)/(CFSE^{low}/CFSE^{high} ratio from vaccinated mouse)]}×100%.

Statistical analyses: Statistical analyses were performed using Graph Pad Prism v8 (San Diego, Calif.). Data from vaccination experiments were analyzed by one-way independent ANOVA, followed by Tukey's or Dunnett's post-hoc testing. Differences were considered significant if p<0.05.

### Results and Discussion

Fabrication of dissolving undercut microneedle arrays: Penetration, dissolution, and delivery efficiency are parameters relevant to dissolving MNA-mediated cutaneous immunization. These factors depend on MNA design, and microneedle geometry contributes to the success of MNA-based cutaneous drug delivery. A number of different microneedle geometries such as circular, obelisk, and pyramid microneedles have been used for MNA-directed intradermal drug delivery. MNAs with obelisk microneedle geometries may result in better penetration and cutaneous delivery efficiency as compared to those with prevailing pyramid microneedles. Further, localizing biocargo to the skin-penetrating tip portion of the microneedles enhances delivery efficiency. Maximizing intradermal delivery efficiency is particularly important for effective cutaneous immunization to enable skin microenvironment conditioning while minimizing the necessary quantities of expensive vaccine components. These factors were considered when developing novel MNAs and the associated manufacturing strategy.

Here, we introduce a novel design of dissolving microneedles for cutaneous vaccination, which features undercut geometry and biomolecules localized in the needle apex. This undercut microneedle geometry is believed to result in better retention and other practical advantages in skin and non-cutaneous tissues. Undercut microneedles present complex fabrication challenges. Micromolding is a useful method for high-throughput manufacturing of microstructures; however, fabrication of microstructures with undercut features through micromolding requires complex processing steps and precision assembly of separately molded, or machined, microneedle tips and shafts. Here, we present a manufacturing strategy and materials to fabricate dissolving MNAs with undercut features through micromolding, eliminating complicated engineering procedures. Importantly, and counterintuitively, we show that undercut microneedles can be directly removed from the flexible production molds which are reusable for several processing cycles—substantially improving cost and productivity. These results suggest that during the micromolding processes, MNA production molds undergo elastic deflection without permanent deformation, and the mechanical stress distribution (caused by removal forces) is smaller than the strength of the microneedle materials, resulting in failure-free removal of undercut microneedles.

The manufacturing strategy we utilize uniquely enables reproducible fabrication of high-quality, tip-loaded dissolving MNAs with undercut features from different and widely-used dissolving microneedle biomaterials, including CMC/trehalose and PVP/PVA compositions (See, e.g., Bediz et al.; Lee et al., “Dissolving microneedles for transdermal drug delivery”, 2008, Biomaterials, 29:2113-2124; Korkmaz et al.). The manufacturing and processing steps result in the final products for example as shown in FIGS. 8A and 8B. Specifically, the master MNA was fabricated from IP-S photoresist by 3D direct laser writing. IP-S is a specific material designed for 3D laser lithography and provides high resolution and mechanical integrity for micro- and nano-structures. We find that 3D laser lithography based on two-photon polymerization provides an effective means for fabrication of undercut MNA designs with smooth edges and sharp tips (˜2 μm tip radius), and without any unwanted residues (e.g., machining chips). To enable more rapid, parallel fabrication of dissolving MNAs, the master MNA was replicated through a two-step micromolding process. The IP-S master MNA was used to fabricate a flexible PDMS mold through soft-lithography, and the resulting PDMS molds were used to manufacture several VeroWhite MNA replicas through UV-curable micromolding. PDMS is a commonly used elastomer with tunable flexibility and low cost for molding of micro- and nano-structures (Lee et al.; Losic et al.). VeroWhite resin is a wear-resistant, acrylic-based photo-polymer extensively used for 3D Polyjet printers (Lin et al., “3D printed, bio-inspired prototypes and analytical models for structured suture interfaces with geometrically-tuned deformation and failure behavior”, 2014, J. Mech. Phys. Solids, 73: 166-182), which renders it an ideal material for MNA master molds. Six MNA replicas were then assembled into one MNA master mold, and this master mold was used to produce several PDMS MNA production molds. Collectively, these processing steps, along with high geometric capability of 3D direct laser writing, resulted in an effective MNA manufacturing strategy. Furthermore, rapid replication of the 3D printed master MNA using a wear-resistant moldable material improved productivity. Based on these results, it is believed that other undercut features could be achieved with this versatile approach, potentially using materials with different Young's modulus for the elastomer molds or/and polymers with different strength properties for microneedles.

Upon fabrication of the MNA master molds with six MNA replicas, dissolving MNAs that integrate vaccine components in the tip portion of the microneedles were fabricated using the conventional three-stage manufacturing strategy through master mold to production mold to final dissolving MNAs (Bediz et al.; Lee et al.). Dissolving MNAs that incorporated the vaccine components (OVA±Poly(I:C)) in the tip portion of the undercut microneedles were fabricated through the spin-casting process. Total OVA and Poly(I:C) content in microneedles was determined to be 10.15±0.87 μg and 24.29±1.60 μg, respectively. With nominal doses of 10 μg OVA and 25 μg Poly(I:C), average loading errors were 5.8% and 6.1%, respectively. During the microneedle tip-loading process, recovery of excess biocargo from MNA production mold reservoirs prior to drying reduces biocargo waste and enables a higher loading efficiency of 77.8±5.8%, which is especially important when working with more expensive vaccine components. For vaccine experiments, we used MNAs made of CMC and trehalose, two FDA-designated “Generally Recognized as Safe” (GRAS) biomaterials. The water-solubility and mechanical strength of CMC make it a good structural material for MNAs (Lee et al.), while trehalose is a disaccharide known to enhance stability of proteins (Kaushik et al., “Why is trehalose an exceptional protein stabilizer? An analysis of the thermal stability of proteins in the presence of the compatible osmolyte trehalose”, 2003, J. Biol. Chem., 278:26458-26465).

To demonstrate compatibility of our MNA fabrication process and the undercut microneedle geometry with another dissolvable biomaterial composition commonly used in the MNA field, we fabricated some MNAs using a PVP/PVA hydrogel. Additionally, MNAs with undercut microneedles tip-loaded with a red colored model drug (doxorubicin) were fabricated to facilitate imaging and demonstrate compatibility of the fabrication process with small molecule agents. Demonstrated compatibility of the MNA fabrication process and undercut microneedle geometry with different types of cargos and material compositions makes application-driven optimization possible, as materials can be selected based on compatibility with bioactive cargo, dissolution requirements, and/or necessary mechanical properties for insertion into different types of skin (e.g., normal skin vs. psoriatic plaques).

In addition to cutaneous vaccination, these dissolving undercut MNAs can be used for a broad range of intradermal and non-cutaneous (e.g., liver, ocular, and cardiac tissues) drug delivery applications (Li et al., “Rapidly separable microneedle patch for the sustained release of a contraceptive”, 2019, Nat. Biomed. Eng., 3: 220-229; Than et al., “Self-implantable double-layered micro-drug-reservoirs for efficient and controlled ocular drug delivery”, 2018, Nat. Commun., 9: 4433; Tang et al., “Cardiac cell—integrated microneedle patch for treating myocardial infarction”, 2018, Sci. Adv., 4:eaat9365). Through the spin-casting process, biocargo(s) of interest can be located either at the tips of microneedles, as depicted in FIG. 8A, or throughout the entire pyramid region, depending on dose requirements. Furthermore, a number of sequential spin-casting steps can be performed to fabricate high-quality MNAs with undercut microneedles that incorporate multiple cargos in their pyramid regions. As such, the presented approach and novel MNA designs are compatible with single and combination therapies for several cutaneous and non-cutaneous applications. Importantly, same production molds can be re-used to fabricate dissolving MNAs, suggesting that removal of undercut MNAs from flexible PDMS production molds results in elastic deformation for several process cycles without destroying the production molds. For example, different dissolving MNAs were prepared using the same PDMS production mold at the first and twelfth cycles. Furthermore, we are currently capable of fabricating 5000+ MNAs per day in our laboratories, and these fabrication processes can be scaled up using industrial grade manufacturing strategies.

Additive manufacturing (AM), or 3D printing, has proven useful for the preparation of drug delivery systems (Prasad et al., “3D printing technologies for drug delivery: a review”, 2016, Drug Dev. Ind. Pharm., 42:1019-1031; Jonathan et al., “3D printing in pharmaceutics: a new tool for designing customized drug delivery systems”, 2016, Int. J. Pharm., 499:376-394). Indeed, there are currently FDA approved, 3D-printed drug delivery systems, such as Spritam® tablets (Prasad et al.; Jonathan et al.). A unique advantage of AM over traditional subtractive fabrication techniques is the possibility for accurate and reproducible manufacturing of 3D complex geometries without design limitations (Johnson et al.). As such, AM offers a high degree of design flexibility and control, and thus enables rapid design-to-fabrication turnaround for optimal application-driven drug delivery systems (Economidou et al., “3D printing applications for transdermal drug delivery”, Int. J. Pharm., 2018, 544: 415-424). Indeed, micro-scale AM has been effectively used for accurate and reliable fabrication of intradermal drug delivery systems (Johnson et al.). However, the unique advantages of AM have yet to be exploited for scalable fabrication of high-quality dissolving MNAs with novel designs for a broad range of drug delivery applications.

In this study, we utilized the micro-additive manufacturing process, 3D direct laser writing, to enable fabrication of complex, high accuracy, 3D microneedle geometries with smooth edges and sharp tips, as well as undercut features. This technology offers an unprecedented level of flexibility for MNA designs. To demonstrate the range of geometric capability of 3D direct laser writing, we fabricated microneedle designs with diverse geometries. This technology enabled fabrication of a wide range of microneedle geometries with high-fidelity, supporting application-driven optimization. The method it allows a wide range of design changes, including height, width, apex angle, and geometry of the microneedles without requiring complex and custom processing steps. From a strength of materials standpoint, removal of MNAs with different undercut microneedle geometries is governed by needle geometry and material strength, as well as by elasticity and strength of production mold materials. Microneedles with larger undercut geometries may require more flexible production molds with lower Young's moduli for failure-free removal. More flexible PDMS molds can be prepared by adjusting the crosslinker ratio and/or curing temperature (Johnston et al., “Mechanical characterization of bulk Sylgard 184 for microfluidics and microengineering”, 2014, J. Micromech. Microeng., 24: 35017; Khanafer et al., “Effects of strain rate, mixing ratio, and stress-strain definition on the mechanical behavior of the polydimethylsiloxane (PDMS) material as related to its biological applications”, 2009, Biomed. Microdevices, 11:503-508), and hyper-elastic materials, such as Ecoflex, could serve as more flexible alternatives to PDMS (Jeong et al., “PDMS-Based Elastomer Tuned Soft, Stretchable, and Sticky for Epidermal Electronics”, 2016, Adv. Mater., 28:5830-5836), allowing removal of even larger undercut features. Collectively, 3D direct laser writing and use of flexible production molds pave the way for fabrication of a broad range of application-driven MNA designs.

Dissolving undercut MNAs deliver multicomponent vaccines to human skin: To evaluate cutaneous biocargo delivery characteristics of dissolving MNAs with undercut microneedles, MNAs tip-loaded with Allura Red R40 dye were manufactured using the presented fabrication strategy. Allura Red R40 dye-loaded MNAs were applied to living human skin explants and removed after 10 min. Imaging of these MNAs before and after application demonstrated high-quality MNAs and complete dissolution of the microneedles, respectively. The corresponding deposits of MNA-embedded Allura Red R40 dye in the targeted skin was demonstrated.

Successful vaccine delivery through the stratum corneum into the immune cell-rich cutaneous microenvironments is relevant to effective intradermal immunization. To anatomically evaluate the delivery of antigen (OVA) and adjuvant (Poly(I:C)) into human skin, MNAs incorporating both Alexa555-labeled OVA and Alexa488-labeled Poly(I:C) were applied to human skin explants for 10 min and then removed. The targeted human skin was cryo-sectioned and imaged using epifluorescence microscopy. The resulting images demonstrated microneedle cavities penetrating through the epidermis into the dermis, and delivery of fluorescent labeled OVA and Poly(I:C) to targeted human skin microenvironments. Collectively, these results indicate that the MNAs fulfilled the geometric (sharp tips and smooth edges) and mechanical-strength requirements for failure-free human skin penetration (e.g., breaching through the stratum corneum and epidermis), and material requirements for efficient dissolution in the aqueous environment of the skin, thereby presenting an effective cutaneous drug and vaccine delivery platform.

Dissolving undercut MNAs deliver multicomponent vaccines to murine skin: To evaluate cutaneous delivery efficiency and kinetics for undercut MNAs, OVA+Poly(I:C) MNAs were applied to murine abdominal skin for 5, 10, or 20 min, then removed and the remaining biocargo content was measured. Within 10 min, MNAs delivered 80.2%±12.5% OVA and 79.6±5.0% Poly(I:C), with nonsignificant additional delivery for either vaccine component by 20 min. To visually confirm in vivo intradermal multicomponent vaccine delivery in mice, dissolving MNAs with high-fidelity undercut microneedles incorporating both Alexa555-OVA and Alexa488-Poly(I:C) were fabricated as described above. Prior to application, Alexa555-OVA+Alexa488-Poly(I:C) MNAs were imaged using optical stereomicroscopy and epifluorescence microscopy. MNAs were then applied to mice and removed after 10 min. The remaining MNA material after applications was also imaged using optical stereomicroscopy. MNA-treated mice were imaged using the IVIS 200 live animal imaging system with filters for detection of both Alexa488-Poly(I:C) and Alexa555-OVA. MNA-directed co-delivery of OVA (antigen) and Poly(I:C) (adjuvant). Successful in vivo application of dissolvable undercut MNAs to mice, and efficient delivery of both components of a multicomponent vaccine were demonstrated.

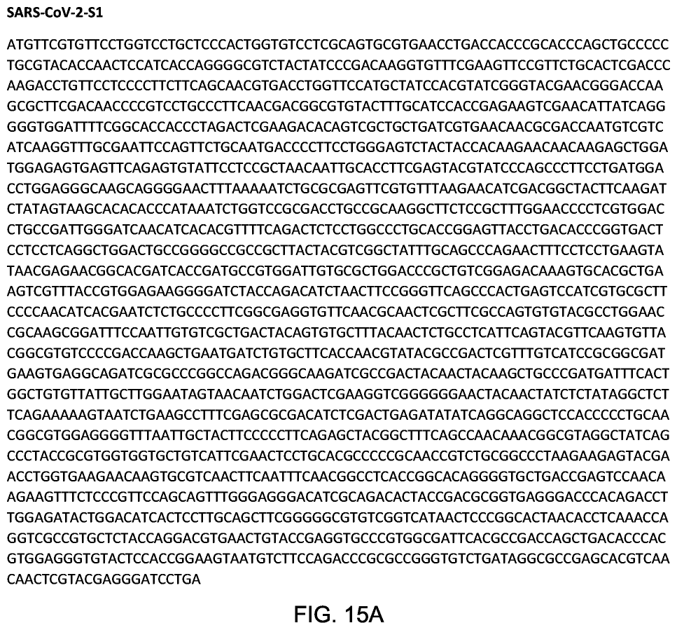

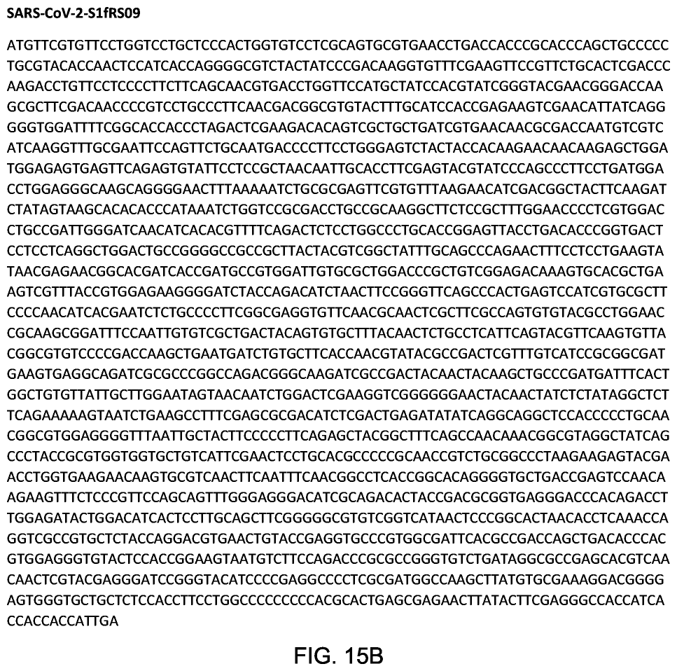

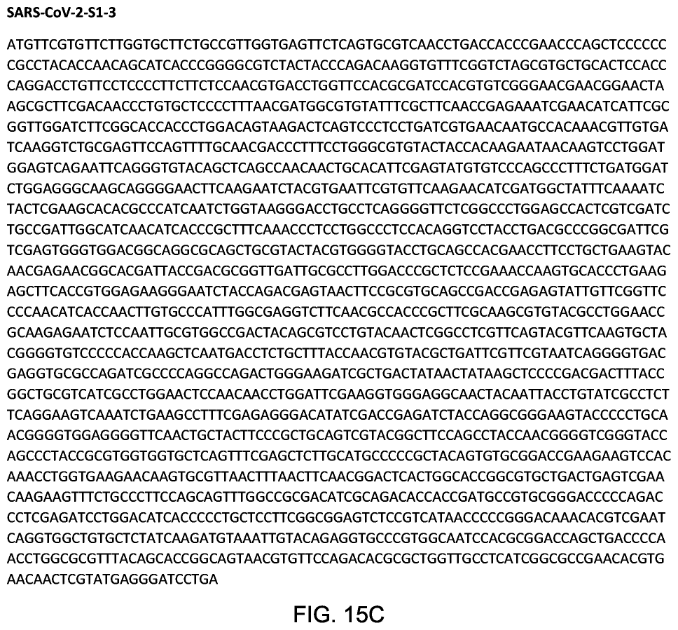

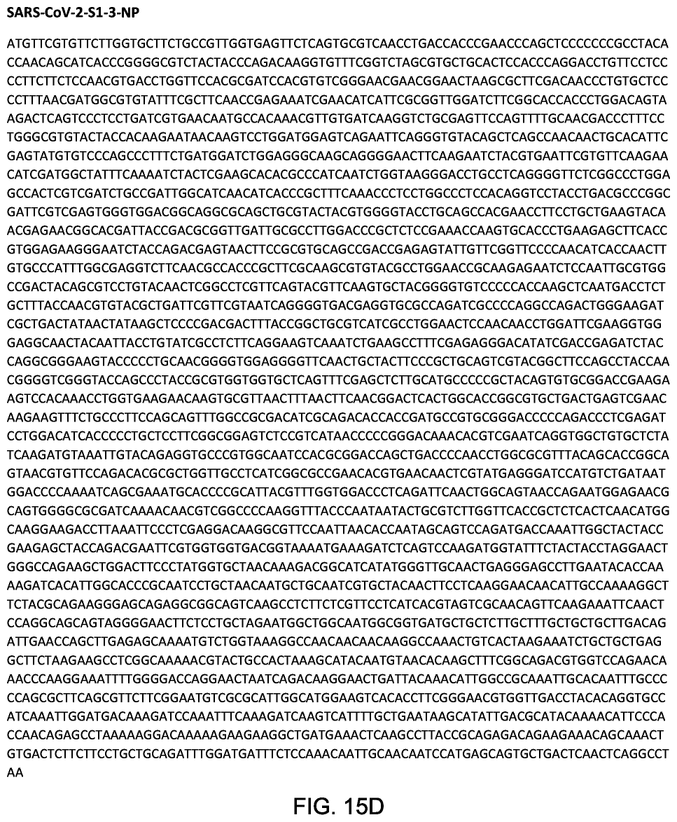

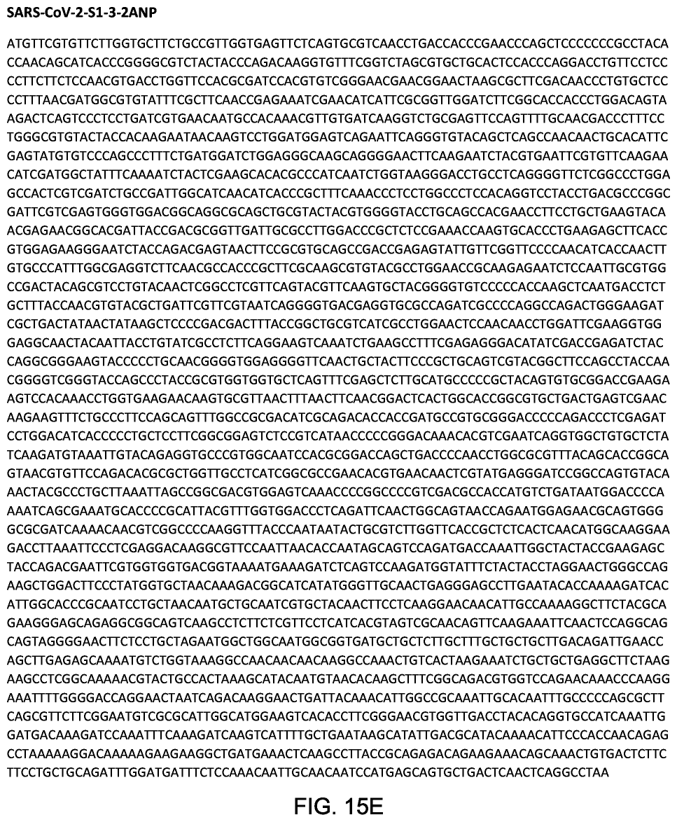

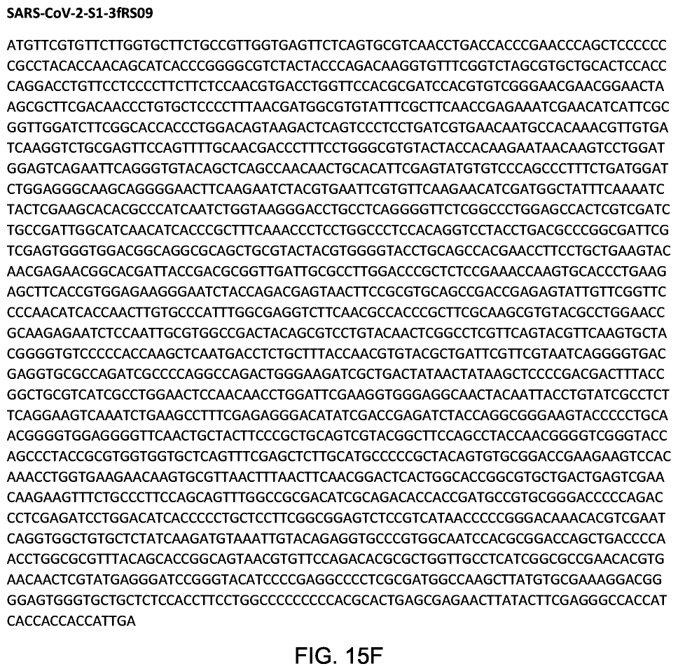

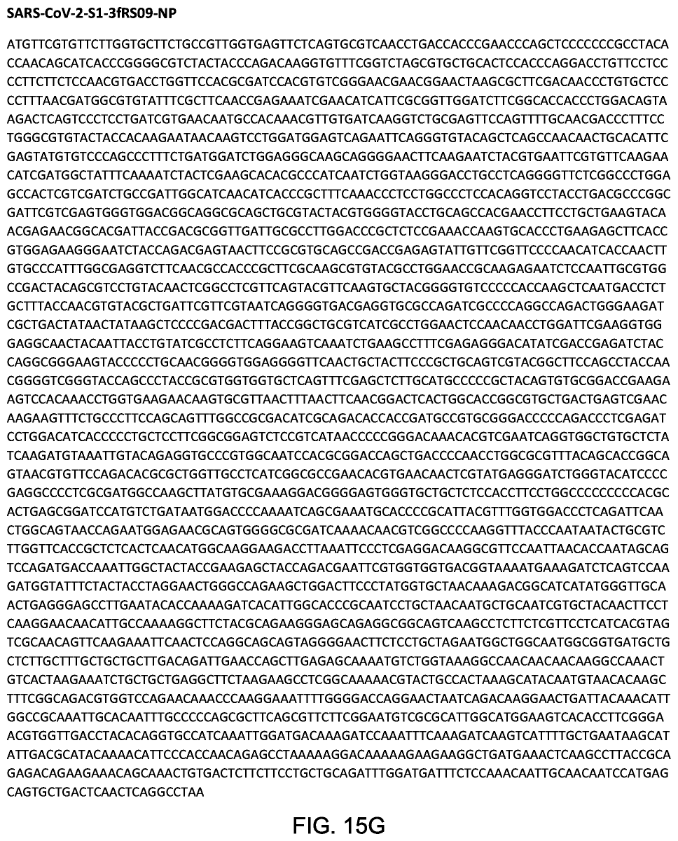

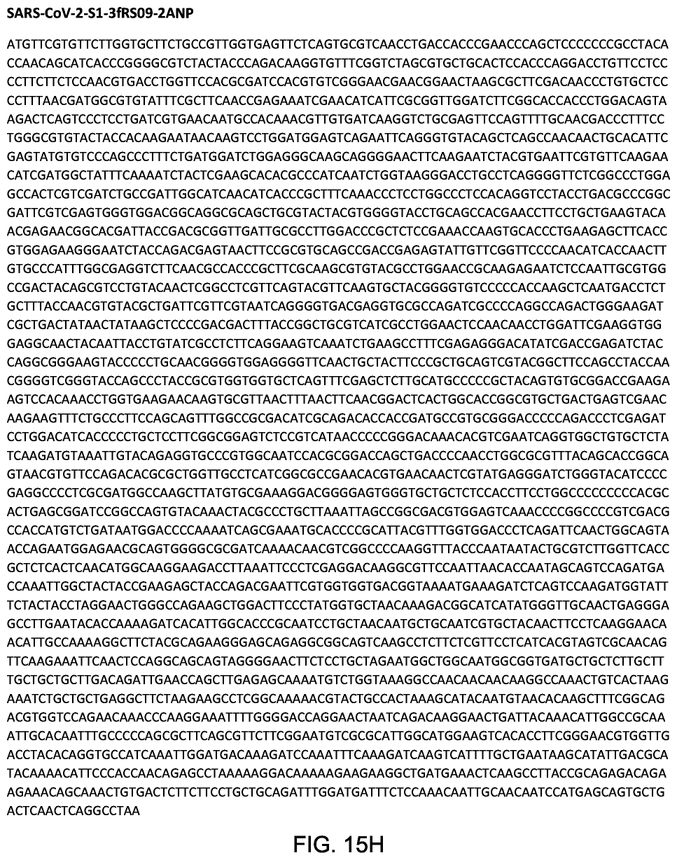

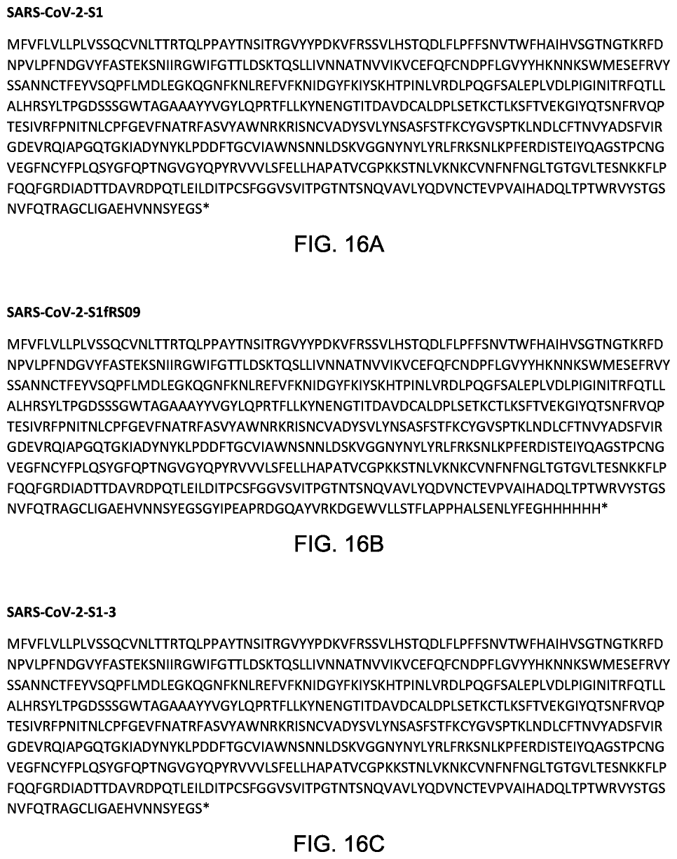

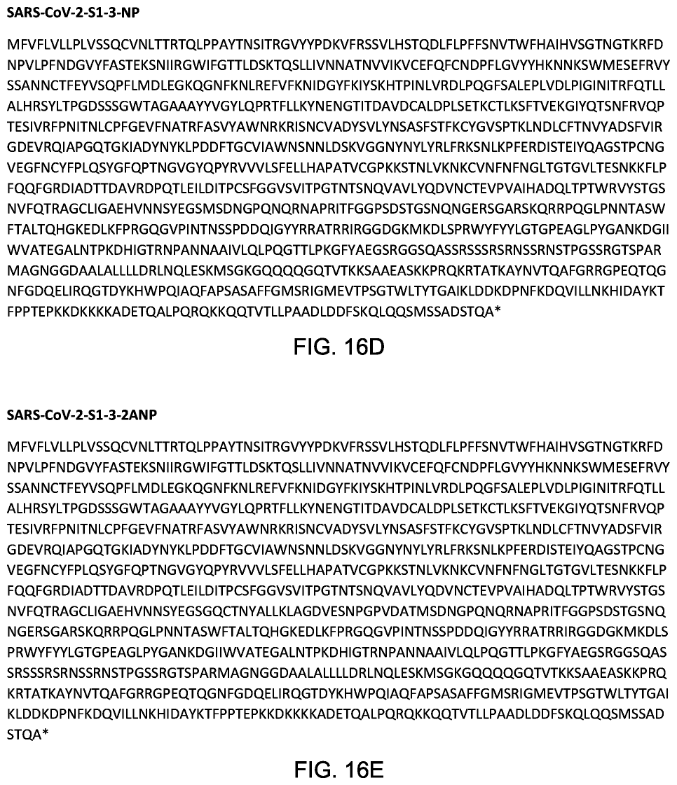

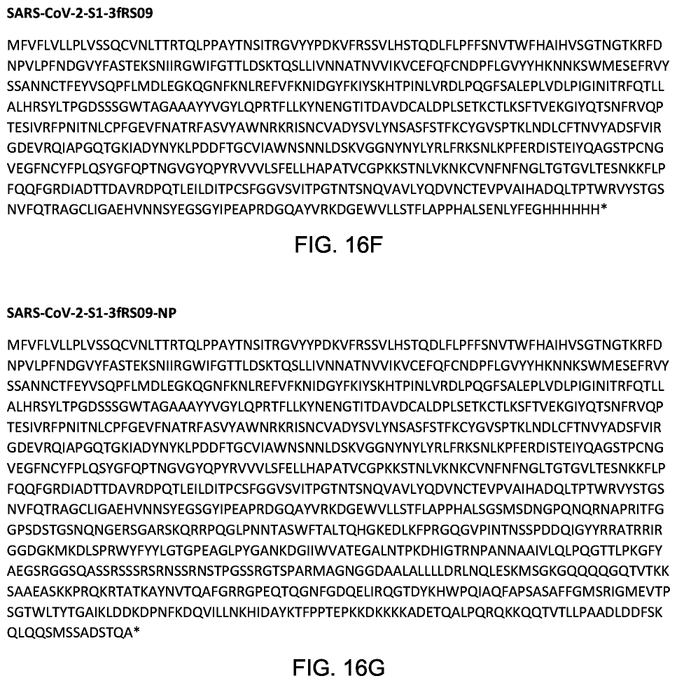

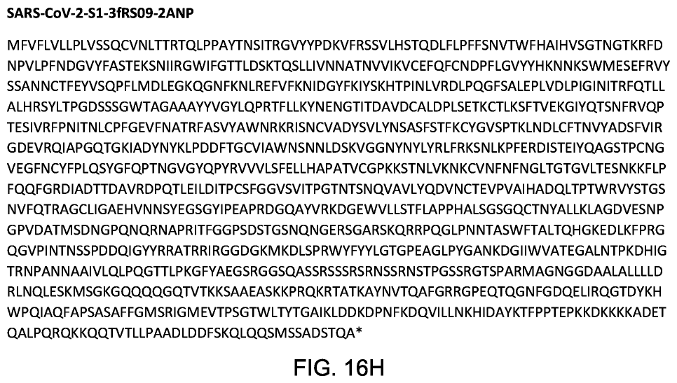

Multicomponent vaccine MNAs induce potent cellular and humoral immunity: Upon demonstration of successful intradermal delivery of the vaccine components to mice, we specifically evaluated immunogenicity of MNA-embedded antigen ±adjuvant and compared MNA immunization to vaccination by the clinically common intramuscular (IM) injection route. To this end, MNAs were fabricated with 10 μg OVA±25 μg Poly(I:C) per MNA, as described above. We and others have previously shown that proteins integrated in dissolving MNAs maintain their integrity (Bediz et al.; Korkmaz et al.). Mice were immunized twice with MNAs or by IM injections, as detailed above, and OVA-specific cytotoxic T-cell (CTL) and antibody responses were quantified using standard in vivo lytic assay and ELISAs, respectively (FIGS. 15A-15C). Notably, dissolving MNAs with different geometries have been used to deliver antigens and other adjuvants (Zhao et al., “Enhanced immunization via dissolving microneedle array-based delivery system incorporating subunit vaccine and saponin adjuvant”, 2017, Int. J. Nanomedicine, 12: 4763-4772; Ding et al., “Microneedle arrays for the transcutaneous immunization of diphtheria and influenza in BALB/c mice”, 2009, J. Control. Release, 136:71-78; McCrudden et al., “Laser-engineered dissolving microneedle arrays for protein delivery: potential for enhanced intradermal vaccination”, 2015, J. Pharm. Pharmacol., 67:409-425), while the Poly(I:C) adjuvant used in this study has only been incorporated in coated or sustained release MNAs (Weldon et al., “Effect of adjuvants on responses to skin immunization by microneedles coated with influenza subunit vaccine”, 2012, PLoS One, 7: e41501; DeMuth et al., “Composite dissolving microneedles for coordinated control of antigen and adjuvant delivery kinetics in transcutaneous vaccination”, 2013, Adv. Funct. Mater., 23: 161-172; DeMuth et al., “Implantable silk composite microneedles for programmable vaccine release kinetics and enhanced immunogenicity in transcutaneous immunization”, 2014, Adv. Healthc. Mater., 3: 47-58).

Cutaneous vaccination with MNAs elicited robust antigen-specific cellular immune responses. As expected, equivalent numbers of antigen-pulsed (CFSE^{high}) target cells and unpulsed (CFSE^{low}) target cells were recovered from spleens of unimmunized mice, indicating the absence of antigen-specific cytolytic activity. Mice treated with Blank MNAs (without antigen or adjuvant) also did not exhibit OVA-specific CTL responses (comparable to naïve). In contrast, specific lysis of antigen-pulsed target cells was dramatically enhanced in immunized mice, as shown by reduced survival of OVA-pulsed targets compared to unpulsed targets. While immunization by IM injections of OVA elicited a relatively low antigen-specific cellular immune response (i.e., OVA-specific lysis), cutaneous vaccination with OVA MNAs led to significantly greater OVA-specific lysis. Importantly, addition of Poly(I:C), a Toll-like receptor 3 (TLR3) agonist adjuvant, to MNAs further improved vaccine immunogenicity, as indicated by a greater CTL response to multicomponent OVA+Poly(I:C) MNAs. The enhanced CTL response we observed with Poly(I:C) adjuvant is consistent with previous reports that TLR3 ligands activate keratinocytes, innate immune cells, and professional APCs and induce cross-presentation of antigen to prime CD8⁺ T cells (Datta et al., “A subset of toll-like receptor ligands induces cross-presentation by bone marrow-derived dendritic cells”, 2003, J. Immunol., 170: 4102-4110; Schulz et al., “Toll-like receptor 3 promotes cross-priming to virus-infected cells”, 2005, Nature, 433:887-892; Kalali et al., “Double-stranded RNA induces an antiviral defense status in epidermal keratinocytes through TLR3-, PKR-, and MDA5/RIG-1-mediated differential signaling”, 2008, J. Immunol., 181: 2694-2704). Given these results with a model antigen, multicomponent cutaneous vaccination using dissolving undercut MNAs that incorporate pathogen- or tumor-specific antigens and adjuvants may be expected to induce robust cellular immunity essential for prevention and/or treatment of many infectious diseases and cancer (He et al., “Skin-derived dendritic cells induce potent CD8⁺ T cell immunity in recombinant lentivector-mediated genetic immunization”, 2006, Immunity, 24: 643-656; Morelli et al.; Condon et al.).

In addition to cellular immunity, cutaneous vaccination with OVA±Poly(I:C) MNAs elicited robust antigen-specific humoral immune responses. While IM immunization resulted in modest OVA-specific serum IgG antibody responses, mice immunized with equivalent doses of OVA antigen in dissolving MNAs had significantly higher IgG levels. Mice not exposed to OVA antigen (i.e., naïve and Blank MNA treated mice) had undetectable levels of OVA-specific antibodies. In particular, we measured serum levels of two subclasses of OVA-specific IgG: IgG1 and IgG2c. Typically, IgG1 antibodies are associated with Th2 type immune responses to extracellular pathogens, while IgG2c antibodies are associated with Th1 type immune responses to viruses and other intracellular pathogens (Nimmerjahn et al., “Divergent immunoglobulin g subclass activity through selective fc receptor binding”, 2005, Science, 310: 1510-1512). Although both IgG subclasses can potentially neutralize and/or opsonize pathogens, IgG2c antibodies can also activate the complement pathway and typically evoke more potent cellular responses because of a greater affinity for activating Fc receptors (FcγRI, FcγRIII, and FcγRIV) and lower affinity for the inhibitory Fc receptor (FcγRIIB) (Nimmerjahn et al.). In our immunization experiments, the addition of Poly(I:C) adjuvant had minimal effect on OVA MNA induced IgG1 responses, but promoted a modest increase in IgG2c responses, consistent with the enhanced CTL immunity. Compared to IM immunization, the stronger and more balanced IgG1/IgG2c responses to cutaneous vaccination with MNAs could translate into enhanced protection against different types of pathogens. Taken together, these results demonstrate that dissolving MNAs with undercut microneedles can efficiently deliver antigens ±adjuvants to APC rich microenvironments within the skin to induce potent cellular and humoral immunity.

### Example 2—Development and Evaluation of an Adenovirus-Vectored SARS-CoV-2 Vaccine

Background: The SARS-CoV-2 coronavirus, which is the causative agent of the COVID-19 pandemic, has spread globally, claiming over 250,000 lives. Although the public-health arsenal against this novel coronavirus is rapidly growing, there is still an urgent need for the development of safe and efficacious SARS-CoV-2 vaccines to combat the COVID-19 outbreak. The spike (S) protein is considered critical for SARS-CoV-2 binding to the cellular receptor angiotensin converting enzyme 2 (ACE2). The S protein, which comprises two subunits (S1 and S2), is thus deemed an ideal target for rational vaccine design. Recombinant DNA technology has emerged as an attractive approach to enabling diverse vaccination strategies. Adenovirus (Ad)-vectored vaccines have been promising for induction of humoral and cellular immune responses.

Methods: Here we constructed and evaluated a recombinant type 5 Ad vector encoding the gene for the antigen SARS-CoV-2-S1 (Ad5.SARS-CoV-2-S1) for vaccination against COVID-19. We immunized BALB/cJ mice with Ad5.SARS-CoV-2-S1 vaccine through subcutaneous (S.C.) injection or intranasal (I.N.) delivery. We assessed the immunogenicity by: 1) measuring SARS-CoV-2-S1-specific antibodies (total IgG, IgG1 IgG2a, IgM) in the sera of vaccinated mice by ELISA; 2) testing the protective ability of the induced antigen-specific antibodies using a plaque reduction neutralization test (PRNT₅₀); and 3) preliminarily evaluating other immune correlates of vaccination-induced protection, including the measurement of antigen-specific antibody-secreting plasma cells in the bone marrow of immunized mice using ELISpot and quantification of SARS-CoV-2-S1-specific IgA antibodies from the lung washes of vaccinated mice using ELISA.

Findings: Results suggest that a single immunization with Ad5.SARS-CoV-2-S1 vaccine (either S.C. or I.N.) elicits significantly higher antigen-specific IgG responses as early as 2 weeks after vaccination compared to unimmunized control groups and also results in antigen-specific long-lived plasma cell responses in the bone marrow of immunized mice. Interestingly, I.N. vaccination induces greater IgG levels than immunization by S.C. injection. Further, mice immunized by I.N. delivery demonstrated increased virus-specific neutralizing antibody responses (emerging on Week 4) compared to S.C. immunized mice and unimmunized controls.

Interpretation: Together, we engineered a clinically-translatable recombinant Ad-based SARS-CoV-2 vaccine candidate and evaluated immune responses to this Ad-vectored vaccine following delivery to mice by S.C. and I.N. routes of administration. Our results demonstrate the promising immunogenicity of the constructed vaccine and support the development of Ad vaccines against COVID-19 and other emerging infectious diseases.

### Materials and Methods

Construction of recombinant adenoviral vectors: The coding sequence for SARS-CoV-2-S1 amino acids 1 to 661 of full-length from BetaCoV/Wuhan/IPBCAMS-WH-05/2020 (GISAID accession id. EPI_ISL_403928) flanked with SalI & NotI was codon-optimized for optimal expression in mammalian cells using the UpGene codon optimization algorithm and synthesized (GenScript). pAd/SARS-CoV-2-S1 was generated by subcloning the codon-optimized SARS-CoV-2-S1 gene into the shuttle vector, pAdlox (GenBank U62024), at SalI/NotI sites. Subsequently, replication-defective human recombinant serotype 5 adenovirus vector, designated as Ad5.SARS-CoV-2-S1, was generated by loxP homologous recombination and purified and stored.

SDS-PAGE and western blot: To evaluate infectivity of the constructed recombinant adenoviruses, A549 (human lung adenocarcinoma epithelial cell line) cells were transduced with a multiplicity of infection of 10 (MOI=10) of Ad5.SARS-CoV-2-S1. At 6 hours after infection, cells were washed three times with PBS, and then incubated with serum-free media for 48 hours. The supernatants of A549 cells infected with Ad5.SARS-CoV-2-S1 were subjected to sodium dodecyl sulfate polyacrylamide gel electrophoresis (SDS-PAGE) and Western blot. Briefly, after the supernatants were boiled in Laemmli sample buffer containing 2% SDS with beta-mercaptoethanol ((3-ME), the proteins were separated by Tris-Glycine SDS-PAGE gels and transferred to nitrocellulose membrane. After blocking for 1 hour at room temperature with 5% non-fat milk in PBST, rabbit anti-SARS-CoV spike polyclonal antibody (1:3000) (Sino Biological) was added and incubated overnight at 4° C. as primary antibody, and horseradish peroxidase (HRP)-conjugated goat anti-rabbit IgG (1:10000) (Jackson immunoresearch) was added and incubated at RT for 2 hours as secondary antibody. After washing three times with PBS, the signals were visualized using ECL Western blot substrate reagents and Amersham Hyperfilm (GE Healthcare).

Animals and immunization: BALB/cJ mice (n=5 animals per group) were vaccinated by either S.C. injection or I.N. delivery of 1.5×10¹⁰ viral particles (vp) of AdΨ5 (a null Ad5 vector control), Ad5.SARS-CoV-2-S1, or S.C. injection of PBS as a negative control. Mice were bled from retro-orbital vein at weeks 0, 2, 4, and 6, and the obtained serum samples were used to evaluate antigen-specific antibodies in immunized mice by enzyme-linked immunosorbent assay (ELISA).

Mice were maintained under specific pathogen-free conditions at the University of Pittsburgh, and all experiments with mice were conducted in accordance with animal use guidelines and protocols approved by the University of Pittsburgh's Institutional Animal Care and Use (IACUC) Committee.

ELISA: Sera from all mice were collected prior to immunization (week 0) and every two weeks (week 2, 4, 6) after immunization and tested for SARS-CoV-2-S1-specific IgG and IgM by conventional ELISA. Briefly, ELISA plates were coated with 200 ng of recombinant SARS-CoV-2-S1 protein (Sino Biological) per well overnight at 4° C. in carbonate coating buffer (pH 9.5) and then blocked with PBS containing 0.05% Tween 20 (PBS-T) and 2% bovine serum albumin (BSA) for one hour. Mouse sera were diluted 1:200 in PBS-T with 1% BSA and incubated for two hours. After the plates were washed, anti-mouse IgG-horseradish peroxidase (HRP) (1:2000, SantaCruz) or anti-mouse IgM-HRP (1:5000, Jackson lmmunoresearch) were added to each well and incubated for one hour. The plates were washed three times and developed with 3,3′5,5′-tetramethylbenzidine, and the reaction was stopped with 1M H2504 and absorbance at 450 nm was determined using an ELISA reader (PerkinElmer 2030). For ELISA of IgG1 and IgG2a, mouse sera were diluted 1:100 and 1:50, respectively, in PBS-T with 1% BSA and incubated for 2 hours. After the plates were washed, biotin-conjugated IgG1 and IgG2a (1:1000, eBioscience) and avidin-horseradish peroxidase (HRP) (1:500, PharMingen) were added to each well and incubated for 1 hour. For IgA isotype ELISA, mice were sacrificed at week 6 post immunization, and bronchoalveolar lavage (BAL) fluids were collected with 1 ml PBS. ELISA was carried out sequentially based on a protocol similar to that described above using 100 μl of BAL fluids and anti-mouse IgA-AP (1:1000, Southernbiotech). The plates were washed three times and developed with para-Nitrophenylphosphate (pNPP), and absorbance at 405 nm was determined using an ELISA reader.

Plaque reduction neutralization test (PRNT): Neutralization capacity of vaccination-induced antibodies in the sera of mice against SARS-CoV-2 (German isolate; GISAID ID EPI_ISL 406862; European Virus Archive Global #026V-03883) were tested using a plaque reduction neutralization test with some modifications. Sera were obtained from all mice on weeks 0, 2, 4, and 6 after immunization and serially diluted in 50 μL Dulbecco's Modified Eagle Medium supplemented with NaHCO₃, HEPES buffer, penicillin, streptomycin, and 1% fetal bovine serum. Virus suspension (50 μL containing 400 plaque-forming units) was added to each well and incubated at 37° C. for 1 hour before placing the mixtures on Vero-E6 cells. The mixtures were added to the cells and incubated for 8 hours. After incubation, cells were fixed with 4% formaldehyde/phosphate-buffered saline (PBS) and stained with mouse monoclonal anti-SARS-CoV nucleocapsid antibody (Sino Biological) and a secondary peroxidase-labeled goat anti-mouse IgG1 (Dako). Finally, the signal was developed by using a precipitate forming 3,3′,5,5′-tetramethylbenzidine substrate (True Blue; Kirkegaard and Perry Laboratories) and the number of infected cells per well were counted using an ImmunoSpot Image Analyzer (CTL Europe GmbH, www.immunospot.eu).

ELISpot for antibody-secreting cells: The frequency of SARS-CoV-2-specific antibody producing cells in the bone marrow of each group was determined by ELISpot assay on week 6 after immunization. 4-HBX plates were coated as described for ELISA assays and non-specific binding was blocked with RPMI media containing 5% FCS. Cells were plated at the indicated density and incubated at 37° C. for 5 hours. Secondary Ab (anti-IgG-alkaline phosphatase; Southern Biotech) was detected using 5-bromo-4-chloro-3-indolyl phosphate substrate (BCIP; Southern Biotech) in 0.5% low melting agarose (Fisher Scientific). Spots were counted on a dissecting microscope and numbers of IgG anti-SARS-CoV-2-S1 antibody forming cells detected were calculated per million bone marrow cells. The qualitative representation of ELISpot plate was prepared in Photoshop.

Statistical analysis: Statistical analyses were performed using GraphPad Prism (San Diego, Calif.). Data, except PRNT₅₀ results, were analyzed by one-way ANOVA, followed by Tukey's post-hoc testing. The data from PRNT were analyzed by Kruskal-Wallis test, followed by Dunn's multiple comparisons. Significant differences are indicated by * p<0.05. Comparisons with non-significant differences are not indicated.

### Results

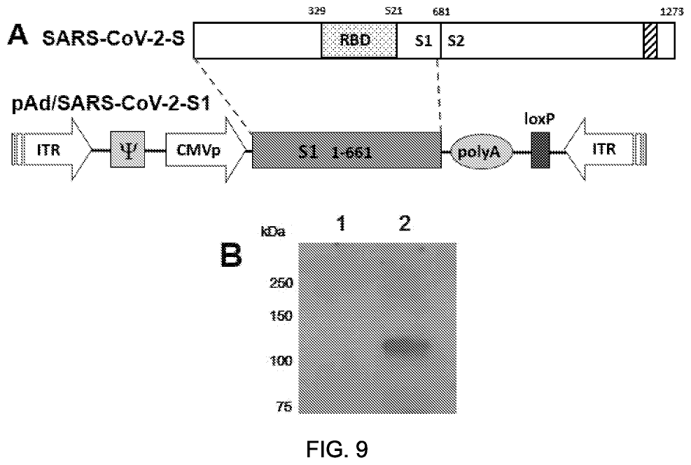

Construction of an adenoviral-vectored SARS-CoV-2 S1 vaccine: To produce E1/E3 deleted replication-defective human type 5 adenovirus expressing SARS-CoV-2-S1 protein, pAd/SARS-CoV-2-S1 was generated by subcloning the codon-optimized SARS-CoV-2-S1 gene into the shuttle vector, pAd (GenBank U62024) at SalI & NotI sites. Subsequently, replication-defective adenovirus 5, designated as Ad5.SARS-CoV-2 S1 (Ad5.S1) was generated by loxP homologous recombination (FIG. 9 (A)). To detect SARS-CoV-2 S1 expression of recombinant adenoviral candidate, the serum-free supernatants from A549 cells infected with Ad5.S1 were characterized by SDS-PAGE and Western blot analysis. The recombinant SARS-CoV-2-S1 proteins were recognized by the polyclonal antibody at the expected glycosylated monomer molecular weights of about 110 kDa, while no expression was detected in the mock-infected cells. (FIG. 9 (B)).

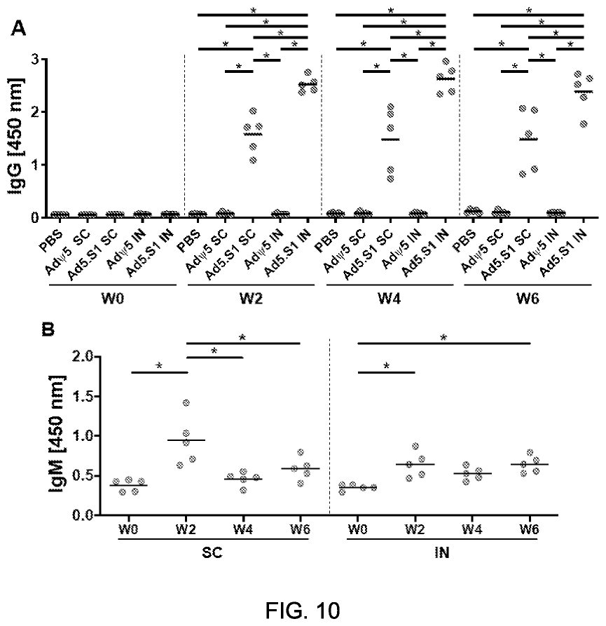

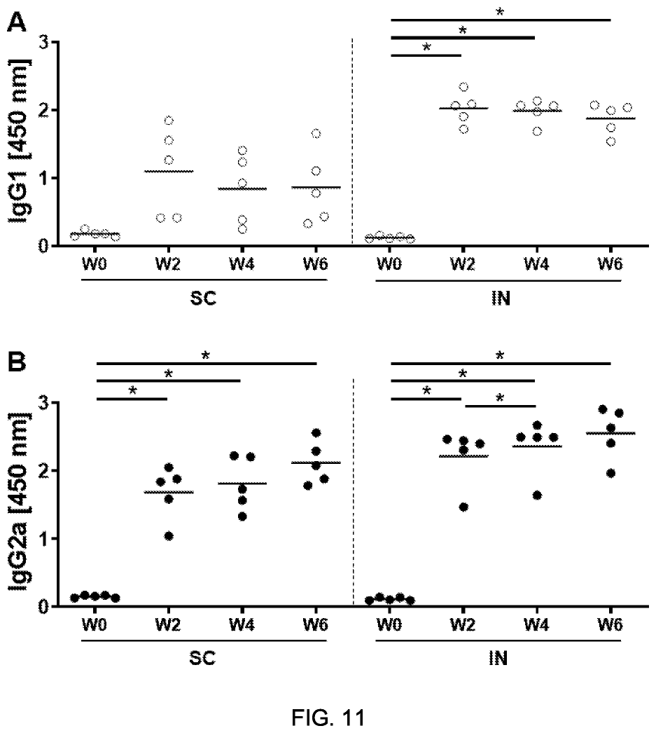

Quantification of SARS-CoV-2-S1 specific IgG and IgM antibodies: To evaluate the immunogenicity of the constructed Ad5.SARS-CoV-2-S1 vaccine, we first measured time-dependent antigen-specific IgG responses in the sera of vaccinated mice (either via I.N. delivery or S.C. injection) and control mice (PBS or Adψ5 immunized groups). Serum samples, collected from all mice before immunization (Week 0) and on Weeks 2, 4, and 6, were used to quantify SARS-CoV-2-S1-specific IgG antibodies for each immunization group using ELISA. Results suggest that as expected, there was negligible antigen-specific IgG antibody responses in the sera of mice vaccinated with the control groups (FIG. 10 (A)). Both I.N. and S.C. immunization induced significant SARS-CoV-2-S1-specific IgG responses as early as 2 weeks after a single vaccination with Ad5.SARS-CoV-2-S1 as compared to control groups (FIG. 10 (A), p<0.05, one-way ANOVA, followed by Tukey's post-hoc testing) and the elicited IgG responses were sustained through Week 6 (maximum length of the study to date). Interestingly, I.N. vaccination with Ad5.SARS-CoV-2-S1 generated higher SARS-CoV-2-S1-specific IgG antibodies than immunization by S.C. injection of the same vaccine. (FIG. 10 (A), p<0.05, one-way ANOVA, followed by Tukey's post-hoc testing). Ad5.SARS-CoV-2-S1 vaccination by both routes of administration also elicited antigen-specific IgM, which emerged within 2 weeks of immunization, compared to pre-immunization (Week 0) (FIG. 10 (B), p<0.05, one-way ANOVA, followed by Tukey's post-hoc testing).

The profile of SARS-CoV-2 S1 specific IgG1 and IgG2a antibodies: To evaluate the quality of IgG responses to Ad5.SARS-CoV-2 S1 vaccine, we determined serum levels of two subclasses of SARS-CoV-2-S1-specific IgG antibodies, IgG1 and IgG2a, in Ad5.SARS-CoV-2-S1 vaccinated groups (either by S.C. injection or via I.N. delivery) using ELISA on Weeks 0, 2, 4, and 6. Intranasal vaccination with Ad5.SARS-CoV-2-S1 elicited significantly higher SARS-CoV-2-S1-specific IgG1 and IgG2a antibody responses compared to pre-immunization group (week 0), which sustained through Week 6 after immunization. (FIG. 11 (A and B), p<0.05, one-way ANOVA, followed by Tukey's post-hoc testing). On the other hand, S.C immunization with the same vaccine induced significantly higher antigen-specific IgG2a responses (FIG. 11 (B), p<0.05, one-way ANOVA, followed by Tukey's post-hoc testing) and negligible IgG1 responses compared to the pre-immunization group. These IgG1 and IgG2a antibody levels suggest that immunization by S.C. injection of Ad5.SARS-CoV-S1 results in Th1 immune responses, whereas immunization through the I.N. route leads to both Th1 and Th2 immune responses to the same vaccine. Further, these results indicate that the choice of the route of Ad vaccine administration may affect the magnitude and type of the ensuing antigen-specific immune response.

Induction of neutralizing antibodies to SARS-CoV-2: To evaluate the functional quality of vaccine-generated antigen-specific antibodies, we used the plaque reduction neutralization assay (PRNT₅₀) to test the ability of sera from immunized mice to neutralize the infectivity of SARS-CoV-2. Sera, collected from all mice prior to immunization (week 0) and on weeks 2, 4, and 6, were tested for the presence of SARS-CoV-2-specific neutralizing antibodies and the results are shown in FIG. 12. As expected, there were no detected neutralizing antibody responses in the sera of mice immunized with PBS or Adψ5 groups (i.e., control groups). Interestingly, there were no detectable neutralizing antibodies in the sera of mice immunized via S.C. injection of Ad5.SARS-CoV-2-S1 vaccine within 6 weeks of immunization, despite significant increases in antigen-specific IgG levels, whereas SARS-CoV-2-neutralizating antibodies were detected in mice immunized by I.N. delivery of Ad5.SARS-CoV-2-S1 on weeks 4 and 6 after vaccination. The resulting SARS-CoV-2-neutralizing activity on week 4 and week 6 after I.N. immunization was statistically significant (FIG. 12, p<0.05, Kruskal-Wallis test, followed by Dunn's multiple comparisons) compared to control groups and S.C. vaccination group. Specifically, BALB/cJ mice vaccinated via I.N. administration of Ad5.SARS-CoV-2-S1 had comparable SARS-CoV-2-neutralizing antibody titers, with the mean values of 76 and 48 on weeks 4 and 6 after immunization, respectively.

Antibody-producing plasma cells in the bone marrow and IgA responses in the lungs: To evaluate the likelihood of generating long-lasting immunity, we performed a preliminary study to investigate antigen-specific antibody-secreting plasma cells in the bone marrow of immunized mice, as measured by ELISPot. Plasma cells in the bone marrow at 6 weeks post-immunization are likely derived from germinal centers and likely to have a long half-life and as such are a desirable correlate of protection. To obtain preliminary evidence of potential mucosal protection, IgA responses in the lungs of immunized mice 6 weeks after immunization were determined using ELISA. This preliminary study was conducted with two mice (n=2) per group to evaluate these important immune correlates of efficacy of Ad vaccination.

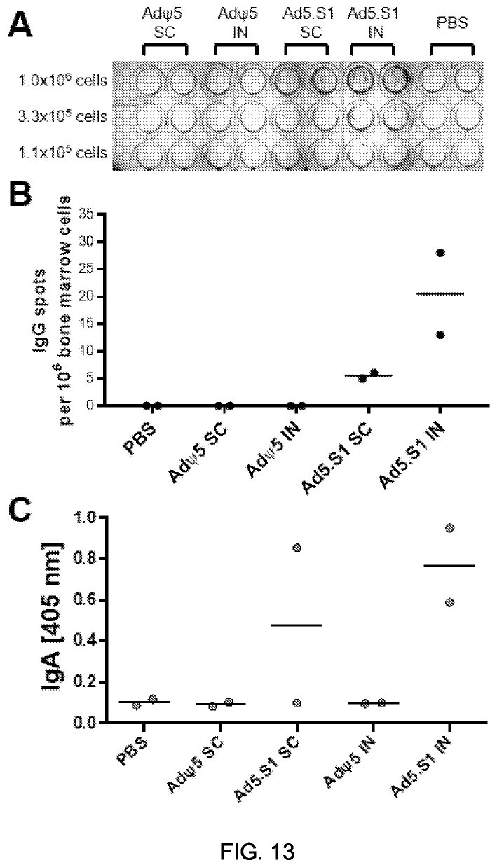

Responses in the antigen-specific antibody-producing ELISpot are reported as spot-forming cells per million input cells in FIG. 13 (B) and the original ELISpot plate corresponding to the quantitative data is shown in FIG. 13 (B). These preliminary results show that S.C. and I.N. immunization with Ad5-SARS-CoV-2-S1 vaccine can yield detectable antigen-specific plasma cells in the bone marrow of immunized mice. Furthermore, IgA responses were detected in bronchoalveolar lavage fluid of mice from those immunized with Ad5.SARS-CoV-2-S1 vaccine delivered by the I.N. route and S.C injection (FIG. 13 (C)). Together, these preliminary studies suggest that our Ad5.SARS-CoV-2-S1 vaccine has the potential to induce bone marrow resident long-lived plasma cell responses and protective mucosal immunity, supporting the further development of Ad-based vaccines against COVID-19.

Our study presents the design and construction of a clinically-feasible adenovirus-vectored-SARS-CoV-2 vaccine (Ad5.SARS-CoV-2-S1) and describes its promising immunogenicity in mice. Our results demonstrate that a single immunization of BALB/cJ mice with the constructed recombinant Ad vaccine delivered by either S.C. or I.N. routes of administration was capable of inducing significantly higher SARS-CoV-2-S1-specific antibody responses as compared to control groups (FIG. 10 (A), p<0.05, one-way ANOVA, followed by Tukey's post-hoc testing). Typically, IgM antibody responses are rapid, and though generally of lower affinity, provide early transient protective immune responses in part due to the pentavalent nature of IgM, whereas induced IgG antibodies dominate later phases of humoral immune responses to ensure sustained prophylactic immunity. In our studies, antigen-specific humoral immune responses, resulting from immunization with Ad5.SARS-CoV-2-S1 vaccine, were evident as early as two weeks after immunization, and IgG levels were sustained through Week 6. After quantifying total antigen-specific IgG responses, a single immunization of BALB/cJ mice with Ad5.SARS-CoV-2-S1 vaccine also elicited SARS-CoV-2-S1-specific IgG subclass responses, IgG1 and IgG2a. Both IgG1 and IgG2a responses were evident as early as 2 weeks after I.N. immunization and sustained through Week 6 (the maximum length of the study to date), whereas only IgG2a responses were substantial with S.C. vaccination (FIG. 11). IgG1 and IgG2a antibodies can be associated with Th2 and Th1 type immune responses, respectively. Accordingly, our results suggest that while both S.C. and I.N. immunization with Ad5.SARS-CoV-S1 vaccine elicit Th1 immune responses, I.N. immunization results in more robust Th2 immune responses. If needed, further improvements could be achieved with different immunization regimens, including homologous or heterologous prime-boost vaccination strategies (K Kardani, A Bolhassani, S Shahbazi. “Prime-boost vaccine strategy against viral infections: Mechanisms and benefits.” Vaccine 2016; 34(4): 413-23; S Y Jung, K W Kang, E Y Lee, D W Seo, H L Kim. “Heterologous prime-boost vaccination with adenoviral vector and protein nanoparticles induces both Th1 and Th2 responses against Middle East respiratory syndrome coronavirus.”Vaccine 2018; 36(24): 3468-76; and S Lu. “Heterologous prime-boost vaccination.”Current Opinion in Immunology 2009; 21(3): 346-51). For instance, cutaneous vaccination with microneedle arrays, which have been shown to deliver a broad range of DNA or protein vaccines with or without adjuvants, could be used to achieve different prime-boost immunization strategies (See, e.g., S C Balmert, C D Carey, G D Falo, et al., “Dissolving undercut microneedle arrays for multicomponent cutaneous vaccination.” Journal of Controlled Release 2020; 317: 336-46; V Bachy, C Hervouet, P D Becker, L Chorro, L M Carlin, S Herath. “Langerin negative dendritic cells promote potent CD8+ T-cell priming by skin delivery of live adenovirus vaccine microneedle arrays.” Proceedings of the National Academy of Sciences of the United States of America 2013; 110(8): 3041-6; N G Rouphael, M Paine, R Mosley, S Henry, D V McAllister. “The safety, immunogenicity, and acceptability of inactivated influenza vaccine delivered by microneedle patch (TIV-MNP 2015): a randomised, partly blinded, placebo-controlled, phase 1 trial.”The Lancet Volume 390, Issue 10095, 12-18 Aug. 2017, Pages 649-658; 390(10095): 649-58; and P C DeMuth, A V Li, P Abbink, J Liu, H Li. “Vaccine delivery with microneedle skin patches in nonhuman primates.” Nature Biotechnology 2013; 31(1082-1085)).

Neutralization assays are pivotal for testing the quality of the immunization-induced antibodies in terms of their ability to reduce the amount of infectious virus titer in culture. In this study, we used the plaque-reduction neutralization test (PRNT₅₀) to test the function of the generated antibodies in the sera of immunized mice. Our results suggest that a single immunization of BALB/cJ mice with I.N. delivery of Ad5.SARS-CoV-S1 vaccine is capable of inducing SARS-CoV-2-neutralizing antibodies as early as 4 weeks after immunization. The ensuing neutralizing activity was significantly greater than that in control groups at weeks 4 and 6 (FIG. 12, p<0.05, Kruskal-Wallis test, followed by Dunn's multiple comparisons). It may be possible to further improve neutralizing antibody responses with different prime-boost vaccination strategies. On the other hand, a single immunization via S.C. delivery of Ad5.SARS-CoV-2-S1 failed to exhibit any significant neutralizing activity at 1:40 dilution with respect to control groups within the 6 weeks of immunization, (FIG. 12, p>0.05, Kruskal-Wallis test, followed by Dunn's multiple comparisons) despite significantly increased levels of IgG (FIG. 10 (A)). Other potential advantages of mucosal immunization via I.N. delivery of Ad vaccines include strong mucosal immune responses against pathogens that initiate infection at mucosal sites, such as SARS-CoV-2. First, unlike painful parenteral injections, I.N. delivery is noninvasive. Second, Adenoviruses infect through mucosal surfaces, with a natural tropism for efficient transduction of cells along the mucosal route. Third, I.N. administration of Ad vaccines can potentially initiate innate immune responses in the respiratory tract against the Ad particles alone, which may precede protection enabled by antigen-specific adaptive immunity induced by the target antigen encoded in Ad vaccines. Fourth, mucosal immunization with Ad vaccines potentially obviates vector-specific immunity to Ad particles. Clinical translation of Ad vaccines has been predominantly hampered by pre-existing immunity against the viral capsid, which diminishes the vaccine efficacy. Thus, I.N. immunization with Ad-based vaccines could be a feasible alternative to combat emerging infectious respiratory diseases, including COVID-19.

Notably, our preliminary evaluation of different important immune surrogates with BALB/cJ mice vaccinated with Ad5.SARS-CoV-2-S1 indicates that a single immunization with the constructed vaccine results in: 1) antigen-specific antibody-secreting plasma cell responses in the bone morrow of immunized mice 6 weeks after immunization (FIG. 13 (A and B), suggesting the existence of long-lived plasma cells ensuring sustained antibody production, and 2) SARS-CoV-2-S1 specific IgA responses in the lungs of vaccinated mice (FIG. 13 (C)), suggesting protective immune responses directed against mucosal pathogens. These results suggest promising immunogenicity of this Ad5.SARS-CoV-2 S1 vaccine.

The developed Ad vaccine induces significant antigen-specific immune responses against SARS-CoV-2 even with a single immunization with relatively low doses, which could be further improved via different prime-boost immunization strategies as needed. These results suggest that adenovirus vaccines are attractive and versatile vaccine candidates to induce virus-specific protective immune responses against COVID-19 and other emerging infectious diseases.

### Example 3—Additional Adenoviral Constructs

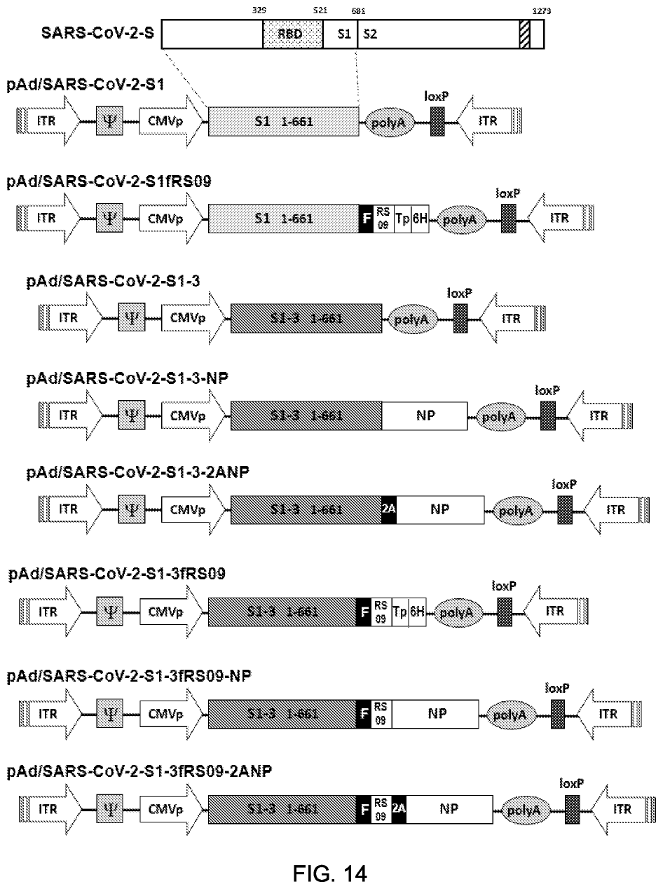

FIG. 14 provides schematic diagrams of various replication-defective adenovirus constructs (genomes) described herein. S1 refers to the SARS-CoV-2 spike protein, with S1-3 being a codon-optimized version thereof (UpGene codon optimization algorithm). CMVp is a constitutive CMV promoter. L is the Ad packaging signal, ITR are Ad inverted terminal repeats. NP is SARS-CoV-2 nucleoprotein. RBD is the receptor binding domain. F is a T4 fibritin foldon trimerization domain. Tp is a Tobacco Etch Virus (TEV) protease. 2A is a self-cleaving peptide sequence. RS09 is a Toll-like receptor 4 (TLR-4) agonist peptide. 6H is a His tag. LoxP is the LoxP recombination site. Constructs were prepared essentially as described above.

### Example 4—Heterogenous Prime-Boost Strategies

BALB/c mice (n=5) were immunized subcutaneously (SC) or intranasally (IN) with 1×10¹⁰ vp of Ad5.SARS-CoV-2-S1N (Ad5.S1N WU), while mice were immunized subcutaneously with PBS as a negative control. For different delivery routes of prime-boost immunization (IN-SC or SC-IN), 1×10¹⁰ vp of Ad5.S1N WU was administered IN or SC three weeks after prime injection. For different antigen for prime-boost immunization (Ad.S1N-subunit, rS1), 15 ug of purified recombinant proteins, Wuhan rS1 or South Africa rS1, were immunized SC or intramuscularly (IM) three weeks or seven weeks after prime injection, respectively. To evaluate the functional quality of vaccine-generated antigen-specific antibodies, mice were bled from retro-orbital vein at weeks 0, 6, 8, and 9 after immunization, and the obtained serum samples were used for microneutralization (NT) assay.

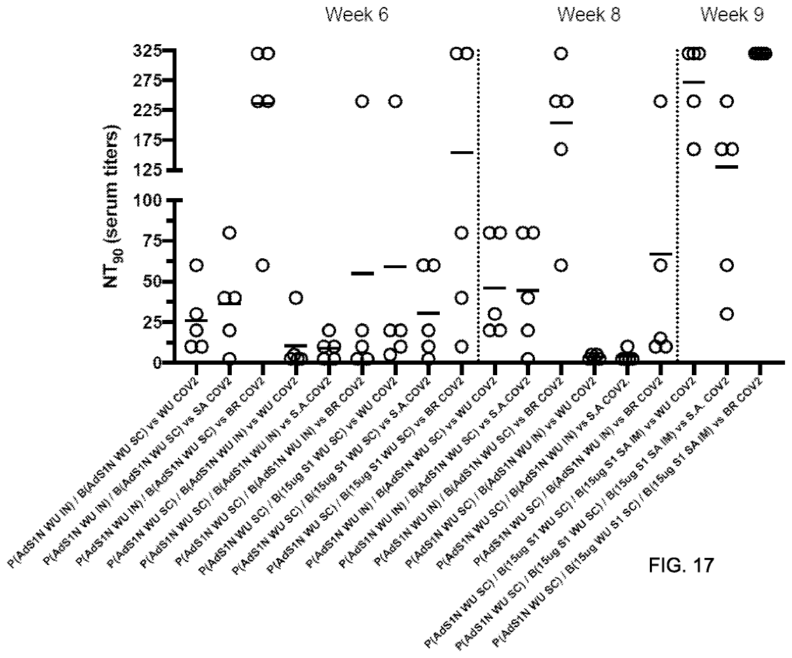

As shown in FIG. 17, SARS-CoV-2-neutralizing antibodies were observed in all groups after immunization at week 6, while there were no detectable neutralizing antibodies in PBS group (data not shown). Interestingly, neutralization against SARS-CoV-2 strain from Brazil (BR COV2) was detected higher than other strains from Wuhan or South Africa (WU COV2 or SA COV2) at all time points. Mice primed-boosted by IN-SC route produced higher levels of SARS-CoV-2-neutralizing antibodies compared to mice primed-boosted by SC-IN route and persisted at least until week 8. Mice primed-boosted by heterologous antigen (Ad.S1N-subunit, rS1) produced high level of SARS-CoV-2-neutralizating antibodies comparable with those from IN-SC immunized mice. Furthermore, mice boosted additionally with South Africa rS1 were showed the highest levels of neutralizing antibodies against all three strains from Wuhan, South Africa, and Brazil. These results suggest that IN-SC delivery route and heterologous prime-boost immunization regimen have a potential impact to gain better immunogenicity and safety of candidate vaccines.

The following animal groups are shown in FIG. 17.

### 1. Control (PBS)

2. Prime: Ad.S1N (1×10¹⁰ vp) IN/Boost: Ad.S1N (1×10¹⁰ vp) SC at week 3.
3. Prime: Ad.S1N (1×10¹⁰ vp) SC/Boost: Ad.S1N (1×10¹⁰ vp) IN at week 3.
4. Prime: Ad.S1N (1×10¹⁰ vp) SC/1st Boost: rS1 Wuhan (15 μg) SC at week 3/2nd Boost; rS1 South Africa (15 μg) IM at week 7.

In further testing, BALB/c mice (n=5) were immunized intramuscularly (IM) with 15 ug of Wuhan rS1 (S1-2cg WU), South Africa rS1 (S1 SA), mixture of Wuhan rS1 and South Africa rS1 (S1-2cg WU, 7.5 ug+S1 SA, 7.5 ug), S1-FRS09, or 1×10¹⁰ vp of Ad5.SARS-CoV-2-S1N (Ad5.S1N), while mice were immunized IM with PBS as a negative control. For homologous prime-boost immunization, 15 ug of Wuhan rS1 (S1-2cg WU), South Africa rS1 (S1 SA), mixture of Wuhan rS1 and South Africa rS1 (S1-2cg WU, 7.5 ug+S1 SA, 7.5 ug), or S1-FRS09 was administered IM three weeks after prime injection. For heterologous prime-boost immunization (S1-2cg WU-S1 SA, Ad5.S1N-S1-2cg WU, Ad5.S1N-S1-S1 SA), 15 ug of Wuhan rS1 (S1-2cg WU) or South Africa rS1 (S1 SA) was immunized IM three weeks after prime injection, respectively. To evaluate the immunogenicity of homologous or heterologous prime-boost immunization, we first determined antigen-specific IgG antibody endpoint titers in the sera of vaccinated mice and control mice (PBS). Serum samples, collected from all mice before immunization (Week 0) and subsequent weeks after vaccination, were serially diluted to determine SARS-CoV-2-S1-specific IgG endpoint titers for each immunization group using ELISA.

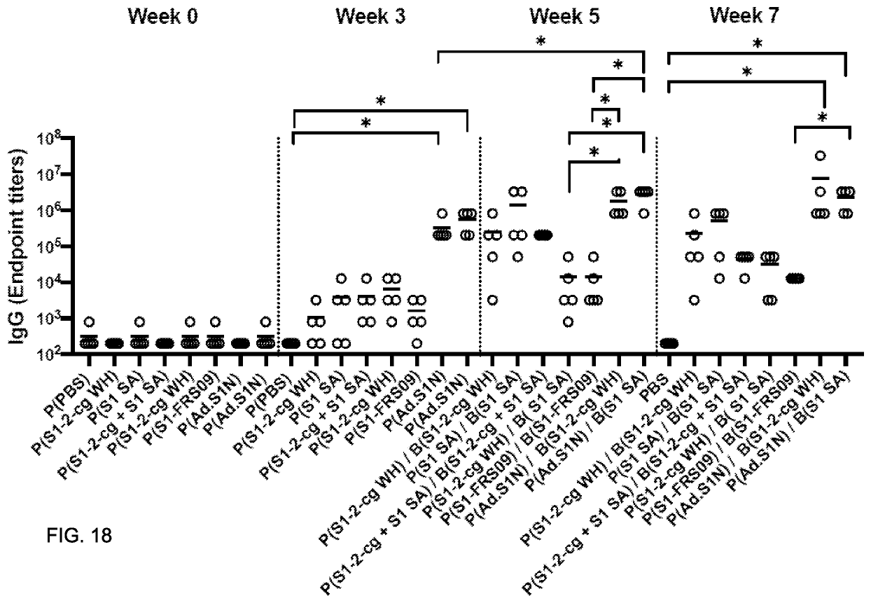

As shown in FIG. 18, even if SARS-CoV-2-S1-specific IgG response was detected in subunit-immunized groups at week 3, 5, or 7 after priming compared to PBS group, no statistically significant difference was observed. As expected, the significant antigen-specific IgG endpoint titers were shown at week 3 after a single vaccination with Ad5.S1N compared to other groups (p<0.05, Kruskal-Wallis test). Interestingly, subunit boost after Ad.S1N prime (Ad5.S1N-S1-2cg WU, Ad5.S1N-S1-S1 SA) exhibited a significant antigen-specific IgG endpoint titer (p<0.05) compared to other subunit-subunit heterologous prime-boost immunization (S1-2cg WU-S1 SA). Together, these results suggest that heterologous prime-boost of Ad5.S1N-subunit is capable of generating robust S1-specific antibody responses. Further evaluation of the functional quality of Ad5.S1N-subunit prime-boost regimen using a plaque reduction neutralization test (PRNT) with three different SARS-CoV-2 strains from Wuhan (WU COV2), South Africa (SA COV2), or Brazil (BR COV2) can be performed.

The following animal groups are shown in FIG. 18.

### 1. Control (PBS).

2. Prime: rS1 Wuhan (15 μg) IM/Homologous Boost (15 μg) IM at week 3.
3. Prime: rS1 South Africa (15 μg) IM/Homologous Boost (15 μg) IM at week 3.
4. Prime: rS1 Wuhan (7.5 μg)+rS1 South Africa (7.5 μg) IM/Homologous Boost (7.5 μg+7.5 μg) IM at week 3.
5. Prime: rS1 Wuhan (15 μg) IM/Boost: rS1 South Africa (15 μg) IM at week 3.
6. Prime: rS1-FRS09 (15 μg) IM/Homologous Boost: rS1-FRS09 (15 μg) IM at week 3.
7. Prime: Ad.S1N (1×10¹⁰ vp) IM/Boost: rS1 Wuhan (15 μg) IM at week 3.
8. Prime: Ad.S1N (1×10¹⁰ vp) IM/Boost: rS1 South Africa (15 μg) IM at week 3.

Having described this invention, it will be understood to those of ordinary skill in the art that the same can be performed within a wide and equivalent range of conditions, formulations and other parameters without affecting the scope of the invention or any embodiment thereof. References incorporated herein by reference are incorporated for their technical disclosure and only to the extent that they are consistent with the present disclosure.

1. A nucleic acid encoding a polypeptide comprising, in frame, a coronavirus S1 protein amino acid sequence and one or both of an epitope string comprising one or more epitopes of a coronavirus polypeptide and/or an amino acid sequence having at least 90% sequence identity with an amino acid sequence of protein of a coronavirus.

2. The nucleic acid of claim 1, wherein the polypeptide comprises an epitope string comprising one or more epitopes of a coronavirus polypeptide.

3. (canceled)

4. The nucleic acid of claim 2, wherein two or more epitopes of the epitope string comprises one or more of the following amino acid sequences: KLWAQCVQL (SEQ ID NO: 16), YLQPRTFLL (SEQ ID NO: 17), LLYDANYFL (SEQ ID NO: 18, ALWEIQQVV (SEQ ID NO: 19), LLLDRLNQL (SEQ ID NO: 20), YLFDESGEFKL (SEQ ID NO: 21), FTSDYYQLY (SEQ ID NO: 22), TTDPSFLGRY (SEQ ID NO: 23), PTDNYITTY (SEQ ID NO: 24), ATSRTLSYY (SEQ ID NO: 25), CTDDNALAYY (SEQ ID NO: 26), NTCDGTTFTY (SEQ ID NO: 27), DTDFVNEFY (SEQ ID NO: 28), GTDLEGNFY (SEQ ID NO: 29), KTFPPTEPK (SEQ ID NO: 30), KCYGVSPTK (SEQ ID NO: 31), VTNNTFTLK (SEQ ID NO: 32), KTIQPRVEK (SEQ ID NO: 33), KTFPPTEPK (SEQ ID NO: 34), VTDTPKGPK (SEQ ID NO: 35), ATEGALNTPK (SEQ ID NO: 36), ASAFFGMSR (SEQ ID NO: 37), ATSRTLSYYK (SEQ ID NO: 38), QYIKWPWYI (SEQ ID NO: 39), VYFLQSINF (SEQ ID NO: 40, VYIGDPAQL (SEQ ID NO: 41), SPRWYFYYL (SEQ ID NO: 42), RPDTRYVL (SEQ ID NO: 43), or IPRRNVATL (SEQ ID NO: 44), separated by spacer amino acids.

5. The nucleic acid of claim 1, wherein the polypeptide comprises an amino acid sequence having at least 90% sequence identity with an amino acid sequence of a coronavirus M protein, N protein, ORF1ab protein, ORF3 protein, an epitope of any of the preceding, or any combination thereof.

6. The nucleic acid of claim 1, wherein the polypeptide further comprises one or more of: a signal peptide, a self-cleaving sequence, a multimerization sequence, or an affinity tag.

7. The nucleic acid of claim 1, wherein the polypeptide ranges from 90% to 110% the length of, and has at least 90% sequence identity with an amino acid sequence of any one of SEQ ID NOS: 11, 12, 13, 14, 15, 58, 59, 60, 61, or 62, or a polypeptide having an amino acid sequence of any one of SEQ ID NOS: 11, 12, 13, 14, 15, 58, 59, 60, 61, or 62.

8. The nucleic acid of claim 1, wherein the polypeptide further comprises a Toll-like receptor agonist domain.

9. The nucleic acid of claim 8, wherein the Toll-like receptor agonist domain is an RS09 domain.

10. The nucleic acid of claim 1, wherein the polypeptide further comprises an angiotensin-converting enzyme 2 (ACE2) receptor-binding domain (RBD) of the SARS-CoV-2 S protein.

11. (canceled)

12. The nucleic acid of claim 1, in the form of a gene incorporated into a replication-defective Adenovirus genome, wherein optionally the genome is a gutless Adenovirus genome.

13. An Adenovirus infectious unit comprising the nucleic acid of claim 12.

14. A delivery device for delivering to a patient a composition comprising the Adenovirus infectious unit of claim 13.

15. The device of claim 14, in the form of a nasal sprayer, and inhaler, an aerosol device, or a nebulizer.

16. The device of claim 14, in the form of a microneedle array comprising a plurality of microneedles.

17-20. (canceled)

21. The device of claim 16, wherein each of the plurality of microneedles comprises trehalose and comprises a tip portion, wherein the Adenovirus infectious unit is contained in the tip portion, and/or wherein the composition comprises a compound with adjuvant or immune stimulation effect.

22. (canceled)

23. (canceled)

24. A method of eliciting an immune response against a coronavirus pathogen in a subject, comprising administering to the subject in a device according to claim 14 a composition comprising the replication-defective adenovirus infectious unit, thereby eliciting the immune response against the pathogen.

25. The method of claim 24, wherein the pathogen is SARS-CoV-2.

26. A method of eliciting an immune response in a patient against a coronavirus, comprising administering to the patient, using the device of claim 14 a composition comprising the replication-defective adenovirus infectious unit, thereby eliciting the immune response against the coronavirus.

27. The method of claim 26, wherein the composition is administered to the patient by inhalation, intra-nasally, or into or through the skin.

28. (canceled)

29. (canceled)

30. A method of generating an immune response to a coronavirus in a patient comprising administering to a patient a first adenovirus infectious unit comprising a genome comprising a gene for expressing an immunogen polypeptide comprising a coronavirus S1 protein amino acid sequence to produce an immune response to the polypeptide.

31-60. (canceled)

61. A polypeptide comprising a coronavirus S1 protein amino acid sequence and one or both of an epitope string comprising one or more epitopes of a coronavirus and/or an amino acid sequence having at least 90% sequence identity with an amino acid sequence of protein of a coronavirus.

62-70. (canceled)
